(19) United States
(12) Patent Application Publication
(10) Pub. No.: US 2002/0182260 A1
(43) Pub. Date: Dec. 5, 2002

(54) Anti-inflammatory agents and methods for their preparation and use

(75) Inventors: Vivien H.W. Mak, Palo Alto, CA (US); Michael L. Francoeur, Cupertino, CA (US); Charles G. Chavdarian, San Ramon, CA (US); Thomas P. Parks, San Mateo, CA (US); Jung-Chung Lee, Sunnyvale, CA (US); Charles Lee, Union City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cellegy Pharmaceuticals, Inc., South San Francisco, CA

(21) Appl. No.: 10/096,968

(22) Filed: Mar. 11, 2002

Related U.S. Application Data
(63) Continuation of application No. 09/322,138, filed on May 28, 1999.
Application No. 09/322,138 is a continuation-in-part of application No. 09/087,744, filed on May 29, 1998, now abandoned.

(51) Int. Cl. A61K 35/78; A61K 35/12

(52) U.S. Cl. 424/522; 424/776

(57) ABSTRACT

This invention provides concentrated inflammation modifiers obtained from various plant, animal, and mineral sources, and methods for the preparation of such concentrated inflammation modifiers. Also provided are methods of preventing and treating inflammation using the concentrated inflammation modifiers. The invention also provides methods of treating inflammation of the skin and mucosal membranes by administering compositions that contain concentrated inflammation modifiers as active ingredients. The methods are useful for treating inflammatory diseases, as well as for reducing or eliminating inflammation that results from transdermal delivery of a drug, or from cosmetics and skin care products. The concentrated inflammation modifiers and methods are also useful for enhancing wound healing and skin aging.

### CROSS REFERENCE OF RELATED APPLICATIONS

This application claims the benefit of U.S. application Ser. No. 09/087,744, filed May 29, 1998, the disclosure of which is incorporated herein by reference in its entirety.

### FIELD OF THE INVENTION

The present invention relates to the field of modulating inflammatory and immune responses. More particularly, the present invention provides methods for the treatment of diseases in which the modulation of T-lymphocyte and/or B-lymphocyte functions will be therapeutically beneficial. Additionally, the present invention provides compositions that include naturally derived anti-inflammatory agents that are useful as medicaments in the treatment of a variety of inflammatory diseases and immune abnormalities, and methods for their preparation. Furthermore, this invention provides methods that can be beneficial to skin-aging.

### BACKGROUND OF THE INVENTION

Inflammation represents a cascade of physiological and immunological reactions that nature has designed as the first cellular response to noxious stimuli in an effort to localize foreign materials and prevent infection or tissue injury. Clinically, inflammation is a primary disease under acute conditions or is a manifestation of underlying pathophysiological abnormalities in chronic disease, characterized by classic signs of redness, pain, swelling and loss of function.

Regardless of the etiology, most forms of inflammation are propagated as a result of the activation of the immune system. In human beings, the immune response is composed of two major mechanisms: cell-mediated immunity and humoral (antibody) immunity (Nossal G J V (1987) N. Engl. J Med. 316: 1320-1325). Both of these responses have a high level of specificity directed to antigenic epitopes expressed on molecular components of infectious agents, foreign (transplant) or transformed (cancer) cells, or even autologous cells (autoimmunity). Peptides derived from endogenous proteins synthesized within cells (e.g., viral infection, malignant transformation, or transplant antigens) are presented on MHC class I complexes, which are expressed by almost all cells of the body. The T cell receptor of CD8⁺ T cells is specific for peptide-MHC class I complexes on the surface of antigen-presenting cells. The recognition and binding of the CD8⁺ T cell receptor to the peptide-MHC class I complex in concert with CD4⁺ T helper cell lymphokines results in generation of cytolytic T cells capable of direct target cell lysis if the target cells display the specific peptide-MHC complex on their surface. Peptides derived from exogenous proteins are taken up by antigen presenting cells and presented on MHC class II complexes to CD4⁺ T-cells.

The humoral immune response is mediated by B lymphocytes and their cell surface receptors (membrane immunoglobulins) that are able to recognize epitopes displayed by intact protein molecules. The generation of an antibody response requires the activation of CD4⁺ helper T cells by the interaction of CD4⁺ T cells and their lymphokines with B cells whose immunoglobulin cell surface receptor has bound a protein antigen (Powrie and Coffman (1993) Trends Pharmacol. Sci. 14:164-168). If this coordinated response occurs, B cells proliferate, and secrete antibody molecules able to bind epitopes on the surface of protein molecules.

The development of an initial (primary) immune response evolves over 8 to 14 days. Part of this response includes the generation of “memory” B and T cells, which provide a long-term system for rapid immune response upon subsequent exposure to antigen.

In principle, the inflammatory and immune responses can be regulated through the use of drugs (Goodman & Gilman's “The Pharmacological Basis of Therapeutics” eds. Hardman et al. Ninth Edition, McGraw-Hill publishing, 1996). Glucocorticoids and aspirin-like drugs (non-steroidal anti-inflammatory agents, NSAIDs) are the most widely used anti-inflammatory agents. NSAIDs are typically used to treat symptoms of inflammation (e.g. pain and fever). Corticosteroids are effective anti-inflammatory agents, having effects on virtually all inflammatory cells, but manifest significant adverse effects, such as inducing Cushingoid features, skin thinning, increased susceptibility to infection, effects on wound healing, and suppression of the hypothalamic-pituitary-adrenal axis. Other anti-inflammatory drugs presently available produce cytotoxic effects that reflect their initial employment as cancer chemotherapeutics, typically anti-neoplastic agents. Such drugs may kill cells indiscriminately, particularly those that proliferate rapidly. Methotrexate, however, is effective in treating rheumatoid arthritis at doses lower than those used to treat cancer (cytoreductive dose). Immunosuppressive agents, such as cyclosporin A and tacrolimus, are effective in preventing allograft rejection, but their use in treating autoimmune diseases has been limited by the development of severe side effects, particularly nephrotoxicity.

In some instances, natural substances can generate an immune or an allergic response. For example, U.S. Pat. No. 4,165,327, which issued to Yamanaka et al., discloses a method of producing lower allergenic lanolin formulations using liquid-liquid extraction of lanolin or a derivative thereof, with a mixed solvent of a non-polar hydrocarbon and a solvent of water-alcohol. As disclosed therein, lanolin can be used as an ointment but it can be allergenic. Using the methods disclosed, the allergenic properties of lanolin are reduced by using a non-polar hydrocarbon extract.

Similar to the teachings of Yamanaka et al., U.S. Pat. No. 4,138,416, which issued to Koresawa et al., discloses fractionation of woolgrease or lanolin into non-polar and polar fractions. The non-polar fractions are nonallergenic whereas the polar fractions are allergenic.

Moreover, U.S. Pat. No. 5,431,924, which issued to Ghosh et al., discloses an anti-inflammatory pharmaceutical composition containing emu oil or a derivative thereof. The anti-inflammatory ingredient of the emu oil is believed to be esterified fatty acids.

In view of the following, what is needed in the art are new and more effective methods of treating inflammation and immune dysfunctions which carry fewer significant and undesirable side effects than previously available methods. Natural products and their derivatives are needed that can treat inflammation conditions and immune dysfunctions. The present invention provides methods and compositions that fulfill these and other needs.

### SUMMARY OF THE INVENTION

Many therapeutic agents cause undesirable inflammation when administered. This inflammation is characterized by classic signs of redness, pain, swelling and loss of function. What is needed are compositions which will reduce or inhibit the inflammation caused by these therapeutic agents. As such, in one aspect, the present invention relates to compositions that contain a therapeutic agent that causes inflammation and a concentrated inflammation modifier. The administration of the therapeutic agent in conjunction with the concentrated inflammation modifier results in an inflammatory response, as measured in an ear swelling assay, that is reduced as compared to the inflammatory response induced by administration of the therapeutic agent in the absence of the concentrated inflammation modifier. Preferably, the inflammatory response is reduced by at least about 30% to about 50% by the inclusion of the concentrated inflammation modifier. The concentrated inflammation modifiers can be obtained, for example, from polar chromatographic fractions of a fat, oil, or wax. The sources of such fats, oils or waxes include, but are not limited to, plants, animals and minerals.

In another aspect, the present invention provides methods of modulating an inflammatory condition or other immune disorder by administering an amount of a composition comprising selected anti-inflammatory fractions or agents from a variety of natural sources, or combinations thereof, that are effective in modulating the condition. Disorders for which the methods are useful include, but are not limited to, rheumatoid arthritis, osteoarthritis, asthma, crystal arthritis, inflammatory bowel diseases, psoriasis, eczema, atopic dermatitis, contact dermatitis, T-cell and B-cell mediated diseases, and disorders that are caused by local inflammatory mediator release. The present invention also provides methods for isolating these fractions and processes for enriching these fractions from their respective natural sources.

In another embodiment, the invention provides compositions that contain a concentrated inflammation modifier that is obtained by chromatographic separation of a wool fat-related material. Also provided are methods of modulating an immune response in a mammal by administering to the mammal a composition that contains a concentrated inflammation modifier. The invention also provides methods of inhibiting proliferation of B- or T-lymphocytes, from peripheral blood or lymphoid organs such as thymus, spleen and lymph nodes, by contacting the lymphocytes with a composition that contains a concentrated inflammation modifier.

In another embodiment, the present invention provides methods for inhibiting IL-2 secretion from a cell by contacting the cell with a composition that contains a concentrated inflammation modifier.

In yet another embodiment, the present invention provides methods for inhibiting TNF-α secretion from a cell by contacting the cell with a composition that contains a concentrated inflammation modifier. In certain aspects, the inflammation modifier interferes with TNF-α secretion.

The present invention provides methods for reducing or eliminating inflammation and/or sensitization that can result from administering an inflammation-inducing drug to the skin or a mucosal membrane. The methods involve administering to the skin or mucosal membrane the drug and a composition that consists essentially of a concentrated inflammation modifier in an amount effective to reduce the drug-induced skin inflammation or sensitization. These methods are useful, for example, when a drug is administered in a topical dosage form such as a transdermal patch or by iontophoresis, and the like. The composition can be administered prior to administration of the drug, after the topical dosage form is removed, or can be administered concurrently with the drug by, for example, including the composition in the topical dosage form.

The present invention also provides methods of treating skin sensitization, and for preventing sensitization that would otherwise be induced by a drug or cosmetic. The methods involve applying to a sensitized area, or to the site of administration of a sensitization-inducing compound, an effective amount of a composition comprising an anti-inflammatory fraction or agent, and a combination thereof.

In another embodiment, the invention provides methods for modulating the healing of a wound by administering to the wound a composition that contains a concentrated inflammation modifier in an amount effective to modulate healing of the wound. The modulating can include enhancing wound healing, as well as preventing the progression of wound development. The compositions can also include one or more oils that are high in unsaturated fatty acids.

The present invention provides methods for minimizing adverse reactions due to application of a topical cosmetic, cosmeceutical, dermatological, or other dosage form. These embodiments involve administering to the site where the dosage form was applied, a concentrated inflammation modifier in an amount effective to reduce the amount of skin or mucosal membrane inflammation induced by the dosage form. Dosage forms for which the methods are useful include, but are not limited to, alpha-hydroxy acids, retinoic acid, retinoic acid derivatives, vitamin D3 and derivatives, and other irritating compounds, as an active ingredient.

In still yet another embodiment, the present invention relates to compositions that contain an inflammation modifier which results in the prevention or reversal of aging. More specifically, compositions which contain inflammation modifiers are effective in preventing and reversing skin aging. These and other embodiments of the present invention will become more apparent when read with the detailed description and the accompanying drawings which follow.

### BRIEF DESCRIPTION OF THE DRAWINGS

FIG. 1 shows a TLC separation of U.S.P. Lanolin. FIG. 1(a) illustrates the results using mixtures of hexanes and ethyl acetate (EtOAc) to chromatograph lanolin. Hexanes:EtOAc ratios were 100:0; 97.5:2.5; 95:5; 92.5:7.5; and 90:10 for lanes 1 through 5, respectively. FIG. 1(b) shows that CCl₄/EtOAc (98:2) gives somewhat better resolution than a hexanes/EtOAc system (97.5:2.5). The darkness of the spots reflects their appearance after H₂SO₄ charring. The cross-hatched spots had UV absorbances.

FIG. 2 shows the fractionation of lanolin using a carbon tetrachloride/ethyl acetate step-gradient. Fractions collected from the column were separated by TLC in hexanes/EtOAc (97.5:2.5).

FIG. 3 shows the fractionation of lanolin using a hexanes/EtOAc step-gradient. The TLC plate shown in FIG. 3(a), which used hexanes/diethyl ether (99:1), indicates the poor fractionation of lanolin using the hexanes/EtOAc gradient. The TLC plate shown in FIG. 3(b), which used hexanes/EtOAc (57:25), shows that fraction 5 may contain a minor amount of the components that make up fractions 4 and 6.

FIG. 4 shows the subfractionation of fraction 1. TLC in hexanes/CH₂Cl₂ (95:5) shows three distinct subfractions can be obtained from the crude fraction.

FIG. 5 shows a two-dimensional thin-layer chromatographic separation of lanolin (FIG. 5A), avocadin (FIG. 5B), lanolin oil (FIG. 5C), and super sterol ester (FIG. 5D).

FIG. 6 is a dose-response curve of lanolin-mediated reduction of TPA-induced inflammation.

FIG. 7 is a dose-response curve showing the effect of lanolin chromatographic fractions 6-9 on LPS-induced B cell proliferation.

FIG. 8 shows the effect of lanolin chromatographic fractions 6-9 on Con A-stimulated T cell proliferation.

FIG. 9 shows the effectiveness of lanolin and related compounds at suppressing inflammation in vivo. FIG. 9A: Comparison of lanolin oil, lanolin wax, lanolin acid, and lanolin alcohol at 5% concentration in the suppression of TPA-induced acute inflammation in mice: ave±sem, n=6 each. FIG. 9B: Comparison of lanolin oil, lanolin wax, lanolin acid and lanolin alcohol at 5% concentration in suppressing DNFB-induced contact hypersensitivity in mice. ave±sem, n=6 each. FIG. 9C: Dose-dependent suppression of TPA-induced acute inflammation by lanolin oils; ave±sem, n=6 each. FIG. 9D: Dose-dependent suppression of DNFB-induced cutaneous contact hypersensitivity by lanolin oil. ave±sem, n=6 each. FIG. 9E: Effect of orally administered lanolin oil on blocking TPA-induced contact dermatitis in mice. FIG. 9F: Effect of orally administered lanolin oil on delayed hypersensitivity in mice.

FIG. 10: shows the effectiveness of certain inflammation modifiers at suppressing inflammation.

FIG. 11: shows the effectiveness of certain inflammation modifiers at suppressing inflammation.

FIG. 12: shows the effectiveness of certain inflammation modifiers at suppressing inflammation.

FIG. 13: shows the effectiveness of certain inflammation modifiers at suppressing inflammation.

FIG. 14: shows the effectiveness of certain inflammation modifiers at suppressing inflammation.

FIG. 15: shows the effectiveness of certain inflammation modifiers at suppressing inflammation.

FIG. 16: shows the effectiveness of certain inflammation modifiers at suppressing inflammation.

FIG. 17: shows an infrared transmittance spectrum of fraction 8A.

FIG. 18: shows an infrared transmittance spectrum of fraction 9A.

FIG. 19: shows an infrared transmittance spectrum of fraction 10A.

FIG. 20: shows an infrared transmittance spectrum of fraction 11A.

FIG. 21: shows an infrared transmittance spectrum of fraction 12A.

FIG. 22: shows inhibition of IL-2 secretion by compositions of the present invention.

FIG. 23: shows inhibition of TNF-α secretion by compositions of the present invention.

FIG. 24: shows various lanolin-derived fractions tested for their ability to enhance the growth of primary human dermal fibroblasts in culture.

FIG. 25: shows a TLC of a sub-fractionation chromatotron method. Fraction 10A can be further separated into 12 sub-fractions.

### DETAILED DESCRIPTION OF THE INVENTION AND PREFERRED EMBODIMENTS

I. Definitions

Unless otherwise stated, the following terms used in the specifications and claims have the meanings given below:

The terms “treatment,” “therapy,” and the like, include, but are not limited to, changes in the recipient's status. The changes can be either subjective or objective and can relate to features such as symptoms or signs of the disease or condition being treated. For example, if the patient notes decreased itching, reduced redness, or decreased pain, then successful treatment has occurred. Similarly, if the clinician notes objective changes, such as by histological analysis of a biopsy sample, then treatment has also been successful. Alternatively, the clinician may note a decrease in inflammatory lesions or other abnormalities upon examination of the patient. This would also represent an improvement or a successful treatment. Prevention of deterioration of the recipient's status is also included by the term. Therapeutic benefit includes any of a number of subjective or objective factors indicating a response of the condition being treated as discussed herein.

“Skin aging” refers to gradual degeneration of the skin structure which can observed visually by loss of clarity, elasticity, suppleness and an increase in roughness, fine lines, wrinkles, and mottled pigmentation.

“Drug”, “pharmacological agent”, “pharmaceutical agent”, “active agent”, and “agent” are used interchangeably and are intended to have their broadest interpretation as to any therapeutically active substance which is delivered to a living organism to produce a desired, usually beneficial effect. In general, this includes therapeutic agents in all of the major therapeutic areas, also including proteins, peptides, oligonucleotides, and carbohydrates as well as inorganic ions, such as calcium ion, lanthanum ion, potassium ion, magnesium ion, phosphate ion, and chloride ion.

The terms “anti-inflammatory agent,” and the like, include, but are not limited to, agents which reduce the extent and/or severity of inflammation and/or other immune responses. Also included are agents that prevent inflammation from occurring that would otherwise be induced in response to a stimulus, for example, a drug or cosmetic that induces inflammation as a side effect. The concentrated inflammation modifiers of the invention typically demonstrate at least 25% inhibition or reduction of inflammation, preferably the agents result in at least about 50%, and more preferably at least about 75% reduction or inhibition of inflammation. In some embodiments, the inhibition is reduced about 30% to about 50%. As used herein, the terms can also include anti-allergens, anti-sensitizers, and anti-irritants.

The term “concentrated inflammation modifier” means a fractionated portion of a fat, wax, oil or combination thereof from a natural source. Concentrated inflammation modifiers are constituent parts of a mixture that are isolated and are present in a purer state after fractionation or chromatography than before fractionation or chromatography. Preferably, the fractionation process results in isolation of the polar fraction. There are fewer constituent parts within a chromatography fraction than in a starting mixture or natural product. Concentration inflammation modifiers are anti-inflammatory agents.

The term “fractionation” means the separation or isolation of components or constituents of a mixture or a natural product.

The term “natural product” as used herein refers to a product derivable from nature.

The term “nonpolar fraction” refers to the fraction which is separated using chromatography using nonpolar or hydrophobic solvents or conditions which mimic nonpolar solvent conditions. Suitable nonpolar solvents include, but are not limited to, chloroform, pentane, hexane, heptane, octane cyclohexane, benzene, toluene and xylene.

The term “polar fraction” refers to the fraction which is separated using chromatography using a polar solvent or hydrophilic solvent or a polar solvent mixed with a nonpolar solvent, or conditions which mimic polar solvent conditions. Suitable solvents include, but are not limited to, methanol, ethanol, propanol, isopropanol, butanol, ethyl acetate and phenol. Polar solvents include a nonpolar solvent mixed with a polar solvent or combinations thereof.

“Pharmaceutically or therapeutically acceptable” refers to a substance which does not interfere with the effectiveness or the biological activity of the active ingredients and which is not toxic to the host, which may be human or animal, to which it is administered.

A “therapeutically effective amount” refers to the amount of an active agent sufficient to induce a desired biological result. That result may be alleviation of the signs, symptoms, or causes of a disease, or any other desired alteration of a biological system.

II. Concentrated Inflammation Modifiers

The invention provides concentrated inflammation modifiers obtained by fractionation of fats, waxes, and oils, or combinations thereof, from various sources. Methods of preparing these anti-inflammatory compositions are also provided.

The concentrated inflammation modifiers of the invention can be prepared from various animal, plant, or mineral sources. Suitable animal sources include, for example, “Zoo lipids”, shark oil, ostrich oil, coral oil, tallow (beef, sheep, horse, pork, etc.), sperm oil, spermaceti, fish oils, beeswax, mink oil, egg yolk oily extract, dairy fats and oils, feather or down oils, fats, waxes (sebaceous secretions from various species of birds, e.g. ducks, geese, chickens, turkeys, etc., wool, hair, or fur fats, waxes, oils (sebaceous secretions from various species of ruminants, e.g. cattle, sheep, goats, llamas, alpaca, deer, etc. The animal source does not include emu, its oil or its derivatives.

In addition to animal sources, the enriched natural lipids of the present invention can be obtained from plant sources. These plant sources include, but are not limited to, Aloe Vera leaf oil, olive oil, castor oil, apricot kernel oil, avocado oil, grain germ oils, camphor, oil of anise, oil of clove, orange oil, peppermint oil, rose oil amyl butyrate, candelilla wax, carnauba wax, carrot oil, soybean oil, shea butter, nut oils (hazelnut, almond, walnut, peanut, kukui, etc.), palm kernel oil, cotton seed oil, 1-carvone, jojoba oil, rape seed oil, vegetable oil, sesame oil, sunflower oil, safflower oil, corn oil, canola oil, castor oil, eugenol, menthol, rice bran oil, rose hip seed oil, coconut oil, cocoa butter, theobroma oil, natural triglycerides, babassu oil, shorea butter, lard, macadamia nut oil, sweet almond oil, meadowfoam oil, black current seed oil, borage seed oil, evening primrose oil, grapeseed oil and wheat germ oil.

Mineral-based sources include, for example, paraffin products (hard paraffin, liquid paraffin, soft paraffin), petrolatum, mineral oil, and the like. The starting materials for preparing the concentrated inflammation modifiers are available commercially, or can be prepared using methods that are well known to those of skill in the art.

To prepare the concentrated inflammation modifiers of the present invention, the waxes, oils, or fats can be fractionated on the basis of polarity. Various chromatographic fractionation techniques are possible. Suitable techniques include, but are not limited to, gravity chromatography, high pressure liquid chromatography, thin layer chromatography, solid phase chromatography, counter-current chromatography, and super critical fluid extraction. Moreover, the chromatography conditions can be normal phase, reverse phase, solvent-solvent extraction, solid phase extraction, and combinations thereof Those skilled in the art will know of other separation conditions suitable for use in the present invention. Chromatographic separation using silica gel is preferable; one example of a suitable gel is a Merck 60 Flash Grade Silica Gel (approximately 240-400 particle size). In a preferred embodiment, the chromatography resin is present in a column.

The waxes, oils, or fats which are used as starting materials for preparation of the concentrated inflammation modifiers are loaded onto the chromatography resin in a non-polar or hydrophilic solvent. The use of a nonpolar solvent results in components of the composition that include polar functional groups being retained by the chromatography resin, while undesired components are not retained. Typically, the chromatography resin is equilibrated with the non-polar solvent prior to loading the fats, waxes, or oils onto the resin. The polarity of the solvent used for loading the composition onto the chromatography resin is preferably equivalent to, or less than, that of about 100% hexanes. Suitable solvents include, for example, 100% hexane, heptane, cyclohexane, and pentane. The particular solvent chosen for loading the fat, wax, and oil onto the resin is chosen so that the polarity of the solvent is such that, upon elution from the column, fractions are obtained that have characteristic components such as those described herein.

After the fat, wax, or oil is loaded onto the chromatography resin, fractions are eluted using one or more solvents having a greater polarity than that of the non-polar solvent used to load the compounds onto the resin. The nonpolar fractions that do not have anti-inflammatory activity are eluted from the column using a solvent having a polarity which is equivalent to, or less than, that of ethyl acetate:hexanes (about 5:95 (all solvent mixtures described herein are vol/vol, unless otherwise specified). If desired, this elution of non-anti-inflammatory fractions can be performed in a stepwise manner to yield intermediate fractions. For example, elution with a solvent having a polarity equivalent to that of dichloromethane:hexanes (about 2.5:97.5) results in a fraction referred to herein as “Fraction 1.” Subsequent elution with a solvent having a polarity equivalent to that of dichloromethane:hexanes (about 5:95 followed by about 10:90) yields “Fraction 2,” and subsequent elutions with solvents having a polarity equivalent to that of ethyl acetate:hexanes (about 2:98) followed by ethyl acetate:hexanes (about 3:97) and ethyl acetate:hexanes (about 5:95) yield Fractions 3, 4, and 5, respectively. Alternatively, one can simply use the more polar solvent to load the starting compound onto the chromatography resin, in which case the non-inflammatory fractions of the compound are not retained on the resin, thus streamlining the preparation of the desired anti-inflammatory fractions. Furthermore, one can first fractionate based on a solvent extraction method, for example, to separate the starting material into either the methanol soluble or hexane soluble fraction before conducting further chromatographic separation. Fractions 1-5 are not concentrated inflammation modifiers.

The fractions having anti-inflammatory activity are then eluted from the chromatography resin using a solvent which has a still greater polarity than that used to elute the non-anti-inflammatory components. For example, the use of an elution solvent having a polarity that is equivalent to, or greater than, that of methanol:ethyl acetate (about 10:90) will elute from the chromatography resin the majority of the anti-inflammatory components of the starting material. As is the case for elution of the non-anti-inflammatory components, the anti-inflammatory components can be eluted in a stepwise manner. For example, after elution with a solvent having a polarity equivalent to that of ethyl acetate:hexanes (about 5:95), an anti-inflammatory fraction referred to herein as “Fraction 6” can be eluted using a solvent having a polarity equivalent to that of ethyl acetate:hexanes (about 10:90). After elution of Fraction 6, Fraction 7 can be obtained by elution with a solvent having a polarity equivalent to that of ethyl acetate:hexanes (about 25:75). Subsequent elution with a solvent having a polarity equivalent to that of ethyl acetate (about 100%) will yield Fraction 8, after which elution with a solvent having a polarity equivalent to that of methanol:ethyl acetate (about 10:90) yields Fraction 9. Each of Fractions 6-9 has anti-inflammatory activity. For use as concentrated inflammation modifier, fraction 8 is of particular interest.

One of skill in the art will recognize that anti-inflammatory fractions that are useful as concentrated inflammation modifiers can be eluted from a chromatography resin using solvents having polarities intermediate to those used to obtain Fractions 6-9. Fractions having anti-inflammatory activity can be obtained using a first solvent that has a polarity that is equivalent to, or greater than, that of ethyl acetate:hexanes (about 5:95) and a second solvent has a polarity which is greater than that of the first solvent. The second solvent, which elutes the desired anti-inflammatory components of the starting material, will often have a polarity which is equivalent to, or less than, that of methanol:ethyl acetate (about 10:90). For example, a fraction having anti-inflammatory activity can be obtained by using as a first solvent ethyl acetate:hexanes (about 7.5:92.5) and discarding the eluant, after which the anti-inflammatory components are eluted using methanol:ethyl acetate (about 5:95). Other combinations are discernable to those of skill in the art.

The fractionation of waxes, oils or fats can be accomplished using chromatography media, including silica gel of different sizes. For example, by using silica gel having 63-200 mesh 60 Å, twelve fractions are obtained (1A-12A), instead of nine as described above. The column elution profile for this embodiment is set forth in Example 3.

In this embodiment, the column elution profile for activity fractions is as follows. A solvent having a polarity equivalent to that of dichloromethane: hexanes (about 2:98) results in a fraction referred to herein as “Fraction 1A.” Subsequent elution with a solvent having a polarity equivalent to that of dichloromethane: hexanes (about 5:95) yields “Fraction 2A”. Subsequent elution with a solvent having a polarity equivalent to that of dichloromethane: hexanes (about 10:90) yields “Fraction 3A,” and subsequent elutions with solvents having a polarity equivalent to that of ethyl acetate: hexanes (about 2:98) followed by ethyl acetate: hexanes (about 2:98) and ethyl acetate: hexanes (about 3:97) yield Fractions 4A, 5A, and 6A, respectively. Subsequent elution with a solvent having a polarity equivalent to that of dichloromethane: hexanes (about 5:95) yields “Fraction 7A”. Nonpolar fractions 1A-7A are not concentrated inflammation modifiers.

Using this alternative embodiment, the fractions having preferred anti-inflammatory activity are then eluted from the chromatography resin starting at “Fraction 8A”. For example, after elution with a solvent having a polarity equivalent to that of ethyl acetate: hexanes (about 10:90), an anti-inflammation fraction referred to herein as “Fraction 8A” can be eluted using a solvent having a polarity equivalent to that of ethyl acetate: hexanes (about 10:90). After elution of Fraction 8A, Fraction 9A can be obtained by elution with a solvent having a polarity equivalent to that of ethyl acetate: hexanes (about 25:75). Subsequent elution with a solvent having a polarity equivalent to that of ethyl acetate: hexanes (about 50:50) will yield Fraction 10A. Subsequent elution with a solvent having a polarity equivalent to that of ethyl acetate (about 100%) will yield Fraction 11A, after which elution with a solvent having a polarity equivalent to that of methanol (about 100%) yields Fraction 12A. Each of Fractions 8A-11A has anti-inflammatory activity. In preferred aspects, Fraction 10A or a combination of Fractions 9A-11A have anti-inflammatory activity.

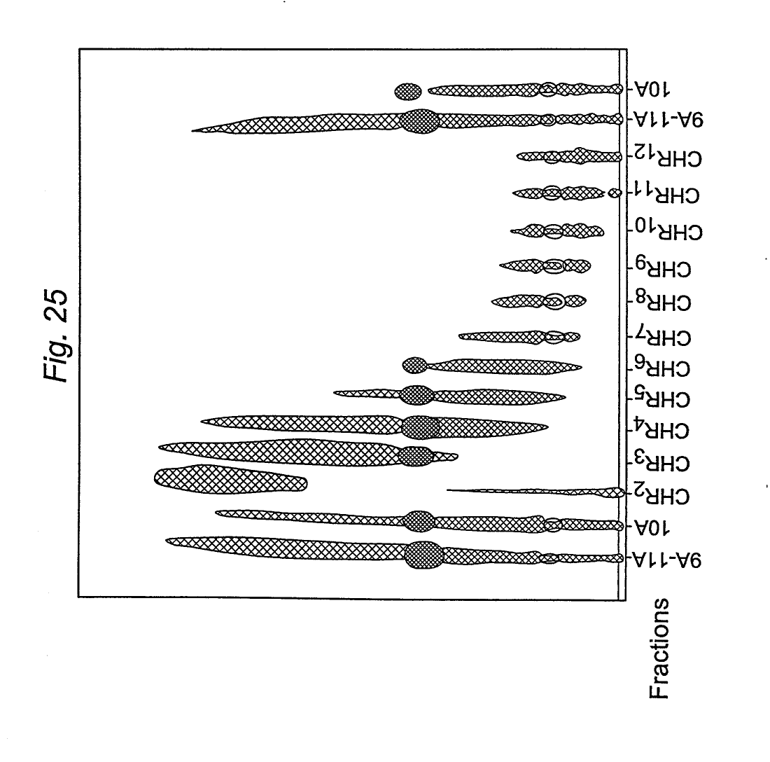

In certain aspects, fractionation cuts can be further separated using various chromatographic techniques. One preferred sub-fractionation method is the chromatotron procedure. With reference to FIG. 25, Fraction 10A can be further separated into 12 sub-fractions (Fractions 10A₁₋₁₂) using a mixed solvent system, such as hexane:ethylacetate (60:40). In this sub-fractionation technique, Fractions 10A₃₋₁₂ show activity.

In another embodiment, fractionation of fats, waxes and oils can be accomplished using solid phase extraction methods. For instance, the oil can be added to a solid support slurry, such as silica gel. A nonpolar solvent can be added and the solution can be optionally heated. The silica gel can then be filtered off, and the nonpolar solvent can be evaporated to provide a nonpolar phase of the oil. The nonpolar fraction is not a concentrated inflammation modifier. A more polar solvent can be added to the filtered silica gel and the slurry can be optionally heated. The slurry is filtered and the polar solvent is evaporated to produce the more polar phase of the natural oil.

In yet another embodiment, lanolin or lanolin oil can be separated using supercritical fluid extraction (SFE). In this technique, lanolin, lanolin oil or a derivative thereof, is charged into a high-pressure extraction vessel. Liquefied gas at a fixed temperature and pressure is introduced into one end of the vessel at a fixed flow rate and removed at the other end enriched in the desirable components. The enriched extraction fluid passes through a pressure reduction valve, which causes the extract to be removed and recovered from the liquefied gas or vapor. The liquefied gas or vapor can be recompressed and reused again in the process. While desirable components may be separated this way, pre- or post-processing can be used to remove undesirable components. For instance odor may be extracted prior to extracting the desirable fraction by using modified pressure and temperature conditions of the liquefied gas media. Sequential processing of the extracts can be used as well.

Suitable liquefied gases include, but are not limited to, carbon dioxide, sulfur dioxide, chlorofluorocarbons, propane, butane, liquefied petroleum gas and mixtures thereof Moreover, organic co-solvents can be used along with the liquefied gas or mixtures of liquefied gases can be used. Those of skill in the art will know of other modifications that can be made to the procedure, such as the addition of silica gel to increase selectivity.

In one preferred embodiment, separation by SFE consists of a two-stage process. The first stage is a deodorization in which liquid carbon dioxide at a pressure ranging from about 1500 psi to about 2600 psi and preferably, about 2200 psi and at a temperature ranging from about 100° C. to about 400° C. and preferably, about 80° C. is passed through the lanolin oil or lanolin until the odor is removed or reduced to the desired level. In the second stage, the extraction of the desired fraction is accomplished using liquid carbon dioxide, which is passed through the deodorized lanolin or lanolin oil obtained from the first stage, at a pressure ranging from about 3200 psi to about 4800 psi and preferably about 4200 psi and at a temperature ranging from about 40° C. to about 100° C. and preferably, about 80° C. until the desirable components showing the biological efficacy are extracted. These desirable components are detecting using TLC and conditions such as pressure and temperature which mimic the polar fractions using column chromatography. The preferred fractions can be compared to the polar fractions of the silica gel columns.

Without intending to be bound by any particular theory, it is thought that the polar fractions of lanolin and lanolin oil may contain some oxysterols in addition to other polar components. Oxysterols are oxygenated derivatives of cholesterol. Examples of oxysterols include, but are not limited to, 7α-hydroxycholesterol, 7β-hydroxycholesterol, 7-ketocholesterol and 25-hydroxycholesterol. As discussed above, the polar fractions are the preferred fractions for biological activity.

III. Inflammatory Diseases, Autoimmune Disorders, and Aging

The invention also provides methods of preventing and/or treating a wide variety of inflammatory diseases, immune disorders, and skin aging. Certain of the diseases for which the methods are effective are discussed herein, as are factors and events which form a theoretical basis for the embodiments of the invention. However, this discussion is not in any way to be considered as binding or limiting on the present invention. Those of skill in the art will understand that the various embodiments of the invention may be practiced regardless of the model used to describe the theoretical underpinnings of the invention.

Arthritis is a collective term for inflammatory disease characterized by pain, swelling and stiffness in the joints and associated tissues. This is usually associated with immunological defects, inappropriate response to microbial antigens, or inflammatory changes provoked by chemical and mechanical damage. Over 200 types of arthritis have been described, of which rheumatoid arthritis, osteoarthritis, seronegative arthritis (e.g., ankylosing spondylitis, which involves the sacro-iliac joints), reactive arthritis and crystal arthritis are the most common. Descriptions of other forms of arthritis can be found in standard medical textbooks such as “Textbook of Medicine” (Eds Wyngaarden et al., 1992). Arthritis may be the dominant symptom of disease, as in osteoarthritis, or one symptom of complex autoimmune diseases such as lupus erythematosus or psoriasis.

Asthma is a chronic respiratory disease characterized by rapid onset of episodes of wheezing, coughing and airway obstruction that are sometimes relieved by bronchodilators or anti-inflammatory agents. It is believed that the inflammation following an initial allergic or toxic stimulus is a key feature of the disease. Eosinophil recruitment and activation is implicated as a central event. Currently, an estimated 25 million people in the US, Europe, and Japan suffer from asthma. It has become the most common chronic disease in industrialized countries and the prevalence, severity and mortality are rising. Allergic asthma is particularly prevalent in children where it may account for 90% of the disease.

Autoimmune diseases arise when the immune system reacts with endogenous proteins that are recognized as “foreign” antigens. This results in the formation of antibodies or immune T cells that can react with these antigens present in tissue to produce destructive changes. Immunosuppressive therapy has been shown to be effective in suppressing autoimmune reactions (Bach (1993) Trends Pharmacol. Sci. 14: 213-216). However, the efficacy of immunosuppressive therapy in the treatment of autoimmune diseases has been variable, and in general it has not been as effective as in organ transplantation or in treatment of specific immune disorders (e.g., the prevention of Rh hemolytic disease of the newborn).

One of the biological consequences of cutaneous inflammatory reactions in the skin is premature aging of the skin, manifested by clinical symptoms such as wrinkles, skin atrophy, abnormal pigmentations, and the like. The best defined cause of inflammatory reactions which leads to skin aging is exposure to UV radiation (UVR). Under acute conditions, sunburn is a clinical manifestation of over-exposure to UV light. Clinically, UVR induces skin redness, edema, and in more severe cases, pain and pruritis. The pathological changes in the skin due to UV exposure have been well-documented in the literature (see, Taylor and Sober (1996) “Sun Exposure and Skin Diseases”, Ann. Rev. Med. 47: 181-191.)

Cutaneous aging is a complex biological process affecting various layers of the skin. It is further complicated by two parallel and yet independent and biologically distinct processes, i.e. intrinsic and extrinsic aging, which affect the skin simultaneously. While intrinsic aging occurs during the passage of time, extrinsic aging is caused by environmental factors such as sun exposure. Ultraviolet radiation triggers a cascade of molecular events in the epidermis and dermis that leads to dermal matrix alterations. Imperfect repair of the degenerated dermal matrix and subsequent UV exposure undermines the structural integrity of the dermis, eventually manifesting as wrinkled and photodamaged skin. Skin inflammation induced by UVR and other environmental stimuli accelerate skin aging.

The hallmark of photodamaged skin is the massive accumulation of elastic fibers in the upper and mid-dermis. This phenomenon is known as “Solar Elastosis” and has been attributed to changes in the composition and organization of elastin and a variety of other extracellular matrix components. Concurrent increased mutations of the p53 tumor suppressor gene and thinning of the epidermis are found in photodamaged skin development (see, Ziegler A., et al., Nature, 372(6508):773-6 (1994)). A less noted changes in photodamaged skin is the presence of a perivenular monocytic-lymphocytic infiltrate in which numerous mast cells are often in juxtaposition to dermal fibroblasts, suggesting that photodamaged skin is chronically inflamed (see, Lavker, R. M. et al., J. Invest. Dermatol., 90(3):325-30 (1988).

UVR is comprised of UVA, UVB and UVC portion of the spectrum. While UVC is almost entirely absorbed by the protective atmospheric ozone layer, UVB can effectively penetrate into the epidermis while UVA reaches deep into the dermal compartment of human skin. UVB affects the epidermis in two separate fashions. It induces cyclobutane pyrimidine dimer formation in DNA and leads to a cascade of events including DNA repair, mutations, and perhaps also cellular transformation. Additionally, the excised DNA photo-product, i.e. the thymidine dimer, stimulates melanogenesis in vitro and in vivo suggesting a protective mechanism to prevent subsequent UV damage. The efficient removal of DNA lesions by cellular repair mechanisms becomes a critical step in the prevention of tumor formation. It is believed that UVB, as tumor initiator and promoter, is responsible for most of the carcinogenic effects of sun exposure.

In addition, UVB is also a potent activator of epidermal cytokine production. Specifically, TNF-α production has been observed in epidermal keratinocytes following exposure to UVB in vitro (see, Kock A., et al., J. Exp. Med., 172(6): 1609-14 (1990), Barker J N W N, et al., Lancet, 337(8735):211-4 (1991), and in human epidermis in vivo after UVB exposure (see, Strickland I, et al., J. Invest. Dermatol., 108(5):763-8 (1997)). The binding of TNF-α to its receptor triggers a signal transduction pathways that leads to nuclear receptor NF-κ activation and translocation to the nucleus where it switches on the transcriptional activity of other genes involved in inflammatory and immune reactions. TNF-α induces the release of other cytokines, e.g. IL-1 (see, Kutsch C. L., et al., J. Invest. Dermatol., 101(1):79-85 (1993)), chemokines, e.g. IL-8, and adhesion molecules, e.g. ICAM-1 (see, Mitra R. S., et al., J. Cell Physiol., 156(2):348-57 (1993) and Middleton M. H., et al., J. Invest. Dermatol., 104(4):489-96 (1995)) and E-selectin (see, Strickland I, et al., J. Invest. Dermatol., 108(5):763-8 (1997)). UVB exposure not only contributes to cancer development in the epidermis, but it is also capable of initiating an inflammatory cascade in the epidermis via TNF-α and IL-1 production, consistent with the clinical observed erythema, pain and swelling of the skin after acute UV exposure.

UVA differs from UVB in that it affects mainly dermal fibroblasts by inducing matrix metalloproteinase (MMP) expression. MMPs are a large family (at least 14 members) of mammalian zinc-dependent endopeptidases that are involved in the degradation and remodeling of the extracellular matrix of connective tissue and basement membranes. With the exception of the membrane-type MMPs (MT-MMPs), all the MMPs are secreted enzymes that degrade extracellular matrix proteins, specifically collagen, fibronectin, elastin, laminin, and proteoglycans. The expression of MMPs play an important pathophysiological role in normal and diseased skin, including tissue remodeling, wound healing, inflammation and skin cancer. Following UVA exposure, upregulation of collagenase (MMP-1, Wlaschek M., et al., Photochem Photobiol., 59(5):550-6 (1994)), gelatinase (MMP-9, Koivukangas V., et al., Acta. Derm. Venereol., 74(4):279-82 (1994)) and stromolysin (see, Fisher G. J., et al., N. Engl. J. Med, 337(20):1419-28 (1997)) has been observed in the skin; localized mainly to dermal fibroblasts. Although most types of MMPs are produced by fibroblasts, the specific MMPs produced by individual cell types can vary. Macrophages, for example, secrete MMP-1, -2, -3, -7, -9, and -12, whereas T cells secrete MMP-2 and MMP-9 (see, D&MD Reports, 1997). Keratinocytes can produce MMP-1 (see, Pilcher B. K., et al., Arch. Dermatol Res., 290 Suppl:S37-46 (1998)), MMP-2 (see, Kratz, G., et al., Br. J. Dermatol. Res., 133(6):842-6 (1995)), and MMP-9 (see, McCawley, L. J., et al., J. Cell Physiol., 176(2):255-65 (1998); and Zeigler, M. E., et al., Invasion Metastasis, 16(1): 11-8 (1996)).

Moreover, up-regulation of MMP-1 (interstitial collagenase, Walschek et al. 1995), MMP-2 & 9 (72-kDa and 92-kDa gelatinases, Koivukangas V., et al., Acta. Derm. Venereol., 74(4):279-82 (1994)) and MMP-3 (45-kDa stromolysin I, Fisher, G. J., et al., N. Engl. J. Med, 337(20):1419-28 (1997)) by fibroblasts and to a lesser extent by epidermal keratinocytes have been observed in vitro and in vivo after UV exposure. The upstream event from MMP up-regulation appears to be the activation and translocation of nuclear receptor AP-1 (see, Fisher G. J., et al., Nature, 379(6563):335-9 (1996)). Since collagen and elastin provide structural integrity to the skin, the induction by UV of MMPs that degrade collagen and elastin in the skin may therefore be the primary mechanism mediating cutaneous photoaging. All-trans-retinoic acid appears to prevent and reverse skin damage due to UV exposure via blocking AP-1 (see, Fisher G. J., et al., Nature, 379(6563):335-9 (1996)). In addition, a reduction of UV-induced collagenase, gelatinase, and stromolysin was detected with retinoic acid pre-treatment. However, retinoic acid did not appear to interfere with UV-induced inflammatory response or NF-κB activation (see, Fisher G. J., et al., Nature, 379(6563):335-9 (1996)).

While it appears that UVA and UVB have distinct roles in initiating skin damage after sun exposure, the signal transduction pathways activated by UVA and UVB are shared by other stimuli. For example, recent studies suggest that UV induction of AP-1 activation can occur via ligand-independent EGF and cytokine receptor (i.e. TNF-α and IL-1 receptors) clustering and internalization (see, Tobin D., et al., Proc. Natl. Acad Sci. U.S.A., 95(2):565-9 (1998)). However, this modest receptor activation by UV can be further amplified by co-administration with their natural ligands such as EGF, TNF-α and IL-1 (see, Rosette C., et al., Science, 274(5290):1194-7 (1996)). As discussed previously, UV is a potent inducer of epidermal cytokine production; it is likely that the UV induction of proinflammatory cytokine release works in concert with the direct UV activation of nuclear factors NF-κB and AP-1 which then contribute to the massive dermal matrix degeneration seen in photoaged skin.

Similar to its response to UV irradiation, the epidermal keratinocyte can initiate and actively participate in the perpetuation of numerous cutaneous inflammatory reactions that follow exposure to a wide array of skin irritants (see, Kock A., et al., J. Exp. Med., 172(6):1609-14 (1990); Ansel J. et al., J. Invest. Dermatol., 94(6 Suppl):101S-107S (1990); Barker J N W N, et al., Lancet, 337(8735):2114 (1991); Nickoloff B. J., et al., J. Am. Acad. Dermatol., 30(4):535-46 (1994) and allergens (see, Piguet P. F., et al., J. Exp. Med, 173(3):673-9 (1991); Griffiths C. E. M. et al., Br. J. Dermatol, 124(6):519-26 (1991); Bromberg J. S., et al., J. Immunol., 148(11):3412-7 (1992); Enk A. H., et al., Proc. Natl. Acad. Sci. U.S.A., 89(4): 1398-402 (1992); Webb E. F., et al., J. Invest. Dermatol., 111(1):86-92 (1998)). Upon stimulation, keratinocytes, the major cell type of the epidermis, express a panoply of proinflammatory cytokines (viz., TNF-α and IL-1), chemokines (e.g. IL-8), and the adhesion molecule ICAM-1, which collectively facilitate the recruitment and retention of infiltrating inflammatory cells into the epidermis. For example, within 2-24 hours following poison ivy exposure, rapid epidermal TNF-α expression is observed in sensitized human volunteers (see, Griffiths C. E. M. et al., Br. J. Dermatol., 124(6):519-26 (1991); Chu C. Q., et al., Clin. Exp. Immunol., 90(3):522-9 (1992); Webb E. F., et al., J. Invest. Dermatol., 111(1):86-92 (1998)). In parallel with the clinical symptoms of irritant contact dermatitis induced by 10% sodium lauryl sulfate, an 8-10 fold increase in TNF-α levels is observed in the peripheral skin lymph nodes (see, Hunziker T., et al., Br. J. Dermatol., 127(3):254-7 (1992)). Barrier disruption induced by tape stripping; either in mice (see, Wood et al. 1992) or human volunteers (see, Nickoloff B. J., et al., J. Am. Acad. Dermatol., 30(4):535-46 (1994)) stimulates cytokine production in the epidermis. While it appears that TNF-α expression precedes IL-1 and IL-8 production in both irritant contact and allergic contact dermatitis, it is also clear that cutaneous inflammation is orchestrated by the initial keratinocyte activation triggered by various environmental stimuli. TNF-α has been implicated in many acute and chronic inflammatory diseases, e.g. psoriasis, rheumatoid arthritis, inflammatory bowel disease, etc.

Since MMP up-regulation can be triggered by TNF-α and IL-1 binding to their respective receptors that are expressed on the cellular surface of keratinocytes and dermal fibroblasts, it is possible that cutaneous inflammation induced by irritants, allergens, or other environmental challenges could cause similar dermal matrix alterations seen after extensive UV exposure. In fact, interstitial collagenase, 92-kDa gelatinase, and stromolysin expression have been observed in cells within or adjacent to extracellular matrix in skin injuries such as wounds and blisters (see, Oikarinen A., et al., J. Invest. Dermatol., 101(2):205-10 (1993); Saarialho-Kere, U.K., et al., J. Clin. Invest., 94(1):79-88 (1994)).

Based on the above-mentioned scientific evidence, it is clear that cutaneous inflammation is intimately involved in the clinical symptoms seen in acute UV exposed skin and in photodamaged skin. The cellular and molecular events leading to cutaneous inflammation by sun exposure appears to be shared by other environmental stimuli such as irritants and allergens. It follows that blocking cutaneous inflammatory cascade initiated by various environmental stimuli, including UV, may provide the broadest and most direct approach to preventing, perhaps also reversing skin aging.

Hallmark histological features of aged skin include thinning of the epidermis, the acellular nature of the dermis, and loss of undulating interface between the epidermis and dermis. Thus, compositions which can reverse or prevent the development of these histological features of aged or aging skin could reduce the clinically observed wrinkles, fine lines and hyperpigmentation associated with aged skin.

Eczema is a generalized term for a dermatitis in which the causative agents are poorly defined. Eczema presents clinically as lesions of variable size not clearly defined from the surrounding normal skin and which are characterized by itching, redness, and scaling. Atopic dermatitis (AD) is a chronically relapsing form of eczema which appears to be inherited along with other atopic diseases such as asthma. Similarly to eczema, the inflammatory processes involved in the pathogenesis of AD have not been determined although it is usually associated with abnormalities of the immune system. Elevated serum IgE levels have long been considered one of the hallmark features of the disease.

Eczematous dermatitis is not a specific disease entity but a characteristic inflammatory response of the skin. Eczematous dermatitis is sufficiently serious to account for the highest incidence of skin disease. Approximately one third of all patients in the United States seen by dermatologists have eczema. This category of skin disease includes atopic dermatitis, lichen simplex chronicus, prurigo nodularis, stasis dermatitis, nummular eczematous dermatitis, dyshidrotic eczematous dermatitis, seborrheic dermatitis, and “eczematous-like” eruptions often accompanying systemic diseases such as Wiskott-Aldrich syndrome, X-linked agammaglobulinemia, phenylketonuria, ahistidinemia, Hurler's syndrome, Hartnup disease, and acrodermatitis enteropathica. In addition, this category includes eczematous dermatitis caused by allergic contact, photoallergic contact, and polymorphous light-induced eruption, as well as infectious eczematoid dermatitis and eczematous dermatophytosis.

Allergic contact dermatitis (ACD) is often difficult to distinguish from other types of dermatitis at the clinical or histological level, yet it has a very distinct cause and mechanism of induction. Exposure to certain environmental agents can elicit the development of an allergy specific for the sensitizing agent. Examples of sensitizers include plant-derived substances such as those in poison ivy and poison oak, metals such as nickel or chromium, or various other chemicals that may be encountered in an occupational setting. In the case of transdermally delivered drugs presently on the market, clonidine, for example, is known to induce allergic contact dermatitis in a high percentage of its current users. The ACD reaction is marked by an influx of lymphocytes and monocytes into the affected area and is characterized by distinctive swelling, redness, and itching. In contrast to dermatologic diseases such as psoriasis where the etiology is poorly understood, the immunological basis of ACD is known in detail, and the reaction which occurs in humans can be accurately reproduced in animal models.

Irritant contact dermatitis (ICD) is more prevalent than allergic contact dermatitis and can occur as a result of exposure to many different chemicals. While exposure to low levels of irritants may have no effect on the skin, irritant dermatitis occurs when the intensity or duration of the exposure exceeds the repair capacity of the skin or when the chemical elicits a nonspecific inflammatory response.

Additional diseases that can be treated by administration of the anti-inflammatory and immunosuppressive agents of the invention include, but are not limited to, respiratory diseases such as chronic obstructive airway disease and adult respiratory distress syndrome, neurological disorders such as multiple sclerosis and Alzheimer's disease, inflammatory bowel disease, Crohn's disease, ischemic/reperfusion injury, and Type I diabetes, infectious diseases including septic shock, graft-vs-host disease, meningitis, gastritis and enteric infections, acne, and periodontal diseases. As the scientific knowledge of the pathophysiological factors responsible for various diseases progresses, it is anticipated that the utility of anti-inflammatory and immunomodulatory agents will expand beyond the current understanding.

IV. Formulations, Dosages, Active Agents and In vitro and In vivo Models

A. Formulations and Dosages

Typically, the concentrated inflammation modifiers described herein will be in the form of an oral, parenteral, or topical formulation for delivering the agent. The formulation will typically contain the concentrated inflammation modifier, typically in concentrations in the range from about 0.001% to 100%, preferably, from about 0.01% to about 50%; more preferably, from about 0.1% to about 15%, together with a non-toxic, pharmaceutically acceptable carrier. See, e.g., DRUG: FACTS AND COMPARISONS, Published by Facts and Comparisons, A Wolters Kluwer Company (1997) and DERMATOLOGICAL FORMULATIONS: PERCUTANEOUS ABSORPTION, Barry (ed.), Marcel Dekker Inc. (1983). In addition, the local absorption and efficacy of these concentrated inflammation modifier-containing formulations can be further enhanced by incorporating an appropriate amount of vegetable oils or botanical oils containing high unsaturated fatty acids, e.g. safflower oil, olive oil, avocado oil, wheat germ oil, etc. or other chemicals ore means that are known to facilitate absorption and delivery of compounds.

Pharmaceutically acceptable carriers will include water, saline, buffers, and other compounds described, e.g., in the MERCK INDEX, Merck & Co., Rahway, N.J. See, also, BIOREVERSIBLE CARRIERS IN DRUG DESIGN, THEORY AND APPLICATION, Roche (ed.), Pergamon Press, (1987). Various considerations are described, e.g., in Gilman et al. (eds) (1990) GOODMAN AND GILMAN'S: THE PHARMACOLOGICAL BASES OF THERAPEUTICS, 8th Ed., Pergamon Press; NOVEL DRUG DELIVERY SYSTEMS, 2nd Ed., Norris (ed.) Marcel Dekker Inc. (1989), and REMINGTON'S PHARMACEUTICAL SCIENCES, the full disclosures of which are incorporated herein by reference. For standard dosages of conventional pharmacological agents, see, e.g., PHYSICIANS DESK REFERENCE (1997 Edition); and American Medical Association (1997) Drug Evaluations (Subscriptions).

The concentrated inflammation modifiers of the invention can be administered in a variety of forms. These include, for example, solid, semi-solid and liquid dosage forms, such as tablets, pills, powders, liquid solutions or suspensions, liposomes, nasal/aerosolized dosage forms, implants, injectable and infusible solutions. These agents can also be incorporated into various cosmetic and toiletry formulations, either for therapeutic usage, or to mitigate irritation or sensitization accompanying these formulations (See, e.g., Flick E. W. COSMETIC AND TOILETRY FORMULATIONS, 2nd Ed., Noyes Publications, 1989). The preferred form depends on the intended mode of administration and therapeutic application.

Dosage forms for the topical administration of the concentrated inflammation modifiers of this invention include powders, sprays, ointments, pastes, creams, lotions, gels, solutions, patches and inhalants. The active compound can be mixed under sterile conditions with a pharmaceutically-acceptable carrier, and with any preservatives, buffers, or propellants which may be required. Topical preparations can be prepared by combining the concentrated inflammation modifiers with conventional pharmaceutical diluents and carriers commonly used in topical dry, liquid, cream and aerosol formulations. Ointment and creams may, for example, be formulated with an aqueous or oily base with the addition of suitable thickening and/or gelling agents. Such bases may include water and/or an oil such as liquid paraffin or a vegetable oil such as peanut oil or castor oil. Thickening agents which may be used according to the nature of the base include soft paraffin, aluminum stearate, cetostearyl alcohol, propylene glycol, polyethylene glycols, woolfat, hydrogenated lanolin, beeswax, and the like. Lotions can be formulated with an aqueous or oily base and will, in general, also include one or more of the following: stabilizing agents, emulsifying agents, dispersing agents, suspending agents, thickening agents, coloring agents, perfumes, and the like. Powders may be formed with the aid of any suitable powder base, e.g., talc, lactose, starch, and the like. Drops may be formulated with an aqueous base or non-aqueous base also comprising one or more dispersing agents, suspending agents, solubilizing agents, and the like.

The ointments, pastes, creams and gels also can contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof Powders and sprays also can contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The topical pharmaceutical compositions can also include one or more preservatives or bacteriostatic agents, e.g., methyl hydroxybenzoate, propyl hydroxybenzoate, chlorocresol, benzalkonium chlorides, and the like. The topical pharmaceutical compositions also can contain other active ingredients such as antimicrobial agents, particularly antibiotics, anesthetics, analgesics, and antipruritic agents.

One example of a topical formulation contains, in addition to the anti-inflammatory agent, light mineral oil, sorbitol solution, hydroxyoctacosanyl hydroxystearate, methoxy PEG-22/dodecyl glycol copolymer, stearoxytrimethylsilane and stearic alcohol, dimethicone 50 cs, fragrance, methylparaben, edetate disodium, quarterium-15, butylates hydroxytoluene, citric acid (monohydrate) and purified water.

The dosage of a specific concentrated inflammation modifier depends upon many factors that are well known to those skilled in the art, for example, the particular agent; the condition being treated; the age, weight, and clinical condition of the recipient patient; and the experience and judgment of the clinician or practitioner administering the therapy. An effective amount of the compound is that which provides either subjective relief of symptoms or an objectively identifiable improvement as noted by the clinician or other qualified observer. The dosing range varies with the compound used, the route of administration and the potency of the particular compound.

Transmucosal (i.e., sublingual, rectal, colonic, pulmonary; buccal and vaginal) drug delivery provides for an efficient entry of active substances to systemic circulation and reduce immediate metabolism by the liver and intestinal wall flora (See Chien Y. W., NOVEL DRUG DELIVERY SYSTEMS, Chapter 4 “Mucosal Drug Delivery,” Marcel Dekker, Inc. (1992)). Transmucosal drug dosage forms (e.g., tablet, suppository, ointment, gel, pessary, membrane, and powder) are typically held in contact with the mucosal membrane and disintegrate and/or dissolve rapidly to allow immediate local and systemic absorption. These formulations are used along with the concentrated inflammation modifiers of the present invention for reducing or eliminating inflammation of transmucosal membranes.

For delivery to the buccal membranes, typically an oral formulation, such as a lozenge, tablet, wash or capsule will be used. The method of manufacture of these formulations are known in the art, including but not limited to, the addition of a pharmacological agent to a pre-manufactured tablet; cold compression of an inert filler, a binder, and either a pharmacological agent or a substance containing the agent (as described in U.S. Pat. No. 4,806,356); and encapsulation. Another oral formulation is one that can be applied with an adhesive, such as the cellulose derivative, hydroxypropyl cellulose, to the oral mucosa, for example as described in U.S. Pat. No. 4,940,587. This buccal adhesive formulation, when applied to the buccal mucosa, allows for controlled release of the pharmacological agent into the mouth and through the buccal mucosa. The anti-inflammatory agents of the present invention can be incorporated into these formulations as well.

For delivery to the nasal or bronchial membranes, typically an aerosol formulation will be employed. The term “aerosol” includes any gas-borne suspended phase of the pharmacological agent which is capable of being inhaled into the bronchioles or nasal passages. Specifically, aerosol includes a gas-borne suspension of droplets of the compounds of the instant invention, as may be produced in a metered dose inhaler or nebulizer, or in a mist sprayer. Aerosol also includes a dry powder composition of a compound of the pharmacological agent suspended in air or other carrier gas, which may be delivered by insufflation from an inhaler device, for example. For solutions used in making aerosols, the preferred range of concentration of the pharmacological agent is 0.1-100 milligrams (mg)/milliliter (mL), more preferably 0.1-30 mg/mL, and most preferably, 1-10 mg/mL. Usually the solutions are buffered with a physiologically compatible buffer such as phosphate or bicarbonate. The usual pH range is 5 to 9, preferably 6.5 to 7.8, and more preferably 7.0 to 7.6. Typically, sodium chloride is added to adjust the osmolarity to the physiological range, preferably within 10% of isotonic. Formulation of such solutions for creating aerosol inhalants is discussed in Remington's Pharmaceutical Sciences, see also, Ganderton and Jones, DRUG DELIVERY TO THE RESPIRATORY TRACT, Ellis Horwood (1987); Gonda (1990) Critical Reviews in Therapeutic Drug Carrier Systems 6:273-313; and Raeburn et al. (1992) J. Pharmacol. Toxicol. Methods 27:143-159.

Solutions of the pharmacological agent may be converted into aerosols by any of the known means routinely used for making aerosol inhalant pharmaceuticals. In general, such methods comprise pressurizing or providing a means of pressurizing a container of the solution, usually with an inert carrier gas, and passing the pressurized gas through a small orifice, thereby pulling droplets of the solution into the mouth and trachea of the animal to which the drug is to be administered. Typically, a mouthpiece is fitted to the outlet of the orifice to facilitate delivery into the mouth and trachea.

Solutions and aqueous suspensions are the pharmaceutical forms most widely used to administer drugs that must be active on the eye surface or in the eye after passage through the cornea or conjunctiva. To increase bioavailability of drugs, to extend therapeutic efficacy, and to improve patient compliance, various dosage forms have been developed over the years. These include soluble inserts (undergoing gradual dissolution/or surface erosion), insoluble inserts (e.g., medicated contact lenses such as Ocusert®, etc.), gels (e.g., Gelrite®), liposomal and drug delivery via nanoparticles (emulsion, suspension, etc.), and ointment (See Edman, BIOPHARMACEUTICS OF OCULAR DRUG DELIVERY, CRC Press, 1993).

Therapeutic agents include, but are not limited to, anti-infectives such as antibiotics and antiviral agents, analgesics and analgesic combinations, anorexics, antiarthritics, antiasthmatic agents, anticonvulsants, antidepressants, antidiabetic agents, antidiarrheals, antihistamines, anti-inflammatory agents, antimigraine preparations, antimotion sickness preparations, antinauseants, antineoplastics, antiparkinsonism drugs, antipruritics, antipsychotics, antipyretics, antispasmodics including gastrointestinal and urinary, anticholinergics, sympatholytics, sympathomimetrics, xanthine derivatives, cardiovascular preparations including calcium channel blockers and beta-blockers, antiarrythmics, antihypertensives, diuretics, vasodilators including general, coronary, peripheral and cerebral, central nervous system stimulants, cough and cold preparations, decongestants, diagnostics, hormones, hypnotics, immunosuppressives, muscle relaxants, parasympatholytics, parasympathomimetrics, psychostimulants, sedatives, tranquilizers, local anesthetics, opiate agonists, opiate antagonists, and pharmacologically active peptides, polypeptides and proteins. Examples of representative drugs within these classes include, by way of example and not for purposes of limitation, ketoprofen, piroxicam, indomethacin, scopolamine, imipramine, desipramine, nortriptyline, clemastine, chlorpheniramine, diphenhydramine, daunorubicin, chloroquine, quinacrine, chlorpromazine, fluphenazine, perphenazine, propranolol, alprenolol, betaxolol, labetalol, metoprolol, timolol, pindolol, atenolol, tetracaine, prilocaine, buprenorphine, naloxone, naltrexone, phentolamine, phenylpropanolamine, ephedrine, mephentermine, bitolterol, tolazoline, streptomycin, gentamycin, somatropin, somatotropin, somatostatin, insulin, insulin-like growth factor (somatomedins), luteinizing hormone releasing hormone, tissue plasminogen activator, and growth hormone releasing hormone.

In a preferred embodiment, the therapeutic agents are sex hormones which include, but are not limited to, estradiol, diethylstilbestrol, conjugated estrogens, estrone, norethindrone, medroxyprogesterone, progesterone, and norgestrel. Other sex hormones which may be utilized include, but are not limited to, testosterone, methyltestosterone, and fluoxymesterone.

Moreover, inflammation modifiers can be used in conjunction with other known active antiinflammatory agents, such as glucocorticoids, hydrocortisone, vitamin D3, methotrexate, cyclosporine alpha-hydroxy acids, retinoic acid, retinoic acid derivatives as well as others, to enhance their respective antiinflammatory activity and to provide fewer side effects.

B. In vivo Models

Animal models that reflect inflammatory or immune responses and to have predictive ability in assessing the efficacy of various treatments for these disorders can be utilized to evaluate the procedures described herein. Various animal models have been developed for irritant contact dermatitis and allergic contact dermatitis. Selected animal models can also be indicative of efficacy in modulation of a systemic immune response, an example of which is the allergic contact dermatitis model. The majority of these models employ mice or rats, e.g., hairless mice or Balb/c mice. A new hairless strain of guinea pig also has been shown to provide a good alternative for investigating both pharmacology and delivery. See MODELS IN DERMATOLOGY, Maibach and Lowe (eds.) Basel, Karger (1989), which is incorporated herein by reference. Typically, these models involve either the topical or systemic administration of a known irritant (e.g., sodium lauryl sulfate) or allergen (e.g., poison ivy antigen, urushiol) to the animal. A specific treatment regimen can be administered via various routes, e.g. oral, topical, etc., prior to the irritant or allergen challenge, concurrently with, and/or subsequent to the challenge.

Several accepted animal models of skin disease are known, each useful to study different aspects of skin disease, for example, the immediate hypersensitivity reaction, delayed type hypersensitivity reaction, non-immunologic contact urticaria, and the like. See NON-STEROIDAL ANTI-INFLAMMATORY DRUGS: PHARMACOLOGY OF THE SKIN, Henby and Lowe (eds.), Basel, Karger (1989). With respect to skin irritation models in animals, inflammation and hyperplasia can be induced by topical application of an active phorbol ester such as 12-O-tetradecanoylphorbol-13 acetate (TPA) and delayed allergic type contact hypersensitivity can be induced using oxazolone, dinitrochlorobenzene (DNCB), or dinitrofluorobenzene (DNFB) as described in Diezel, et al., J. Invest. Derm., 93: 322 (1989), which is incorporated herein by reference. Further, compromised skin barrier can be modeled by topical acetone or detergent treatment, or by tape stripping. TPA causes epidermal inflammation and hyperplasia by activating protein kinase C, a key regulator of epidermal growth and inflammation. The pathophysiologic alterations to the skin induced by TPA bear many similarities to the pathophysiologic alterations observed in psoriatic skin. Skin challenged with DNCB or DNFB several days after sensitization has been observed to exhibit immunologic reactions similar to those observed in clinical cases of allergic contact dermatitis.

In addition to the use of normal mice where skin inflammation is induced via topical application of a specific stimulating agent, a relevant skin disease model can also be developed in immune-compromised mice such as athymic nude mice (See Fogh J. et al. THE NUDE MOUSE IN EXPERIMENTAL AND CLINICAL RESEARCH Academic Press, 1982) and in severe combined immune-deficient (SCID) mice (See Hendrickson Z. A. “The SCID Mouse: Relevance as an Animal Model System for Studying Human Disease,” Am. J. Pathology, 143: 1511-1522, 1993). In these immune-compromised, or immune-deficient mice, excised human psoriatic lesional skin is transplanted to the back of the animal. Once healed completely, these xenografts can be used to model psoriasis in man. Similar approaches can be applied to the development of other skin diseases, as well as infections.

Traditionally, different aspects of wound healing, such as angiogenesis, wound contraction, and connective tissue rearrangement, have been investigated using in vitro and in vivo models. See CLINICAL AND EXPERIENTAL APPROACHES TO DERMAL AND EPIDERMAL REPAIR: NORMAL AND CHRONIC WOUNDS, Barbul et al. (eds.), Wiley-Liss (1991), which is incorporated herein by reference.

As for UV-induced skin aging, several animal models have been used to test for the efficacy of agents which can interfere with photoaging (see, Kligman L H, “The Ultraviolet-irradiated hairless mouse: A model for photoaging”, J. Am. Acad. Dermatol, 21:623-631, 1989; Darr D et al., “Topical vitamin C protects porcine skin from ultraviolet radiation-induced damage”, Br. .J Dermatol., 127:247-253, 1992; Bissett D L et al., “Photoprotective effect of superoxide-scavenging anti-oxidants against ultraviolet radiation-induced chronic skin damage in hairless mouse”, Photodermatol Photoimmunol Photomed, 56-62, 1990; Kligman L H “The hairless mouse and photoaging” Photochem Photobio., 1990.)

V. Methods and Compositions for Modulating Inflammatory and Autoimmune Disorders

The present invention provides methods and compositions for modulating inflammatory conditions and immune responses of mammals. The methods involve administering a concentrated inflammation modifier which is isolated from naturally sources. These concentrated inflammation modifiers are capable of treating preexisting inflammatory conditions and immune disorders, and also preventing or reducing inflammation that would otherwise be induced by a drug or stimulus administered to the animals. For example, the methods and compositions can prevent or reduce inflammation caused by an inflammation-inducing drug or cosmetic administered transdermally or iontophoretically. Also provided are methods and compositions for modulating wound healing, both enhancing the healing process and preventing the progression of wound development. Unlike previously available methods of preventing or treating inflammation, the methods provided by the present invention are relatively free of undesirable side effects.

In another embodiment, the present invention provides methods for inhibiting IL-2 secretion from a cell by contacting the cell with a composition that contains a concentrated inflammation modifier. For instance, proliferation of T-cells in response to antigen recognition is mediated by an autocrine growth pathway, wherein the T-cell secretes IL-2 and also expresses cell surface receptors for this cytokine. The principal autocrine growth factor for most T-cells is IL-2. Thus, by inhibiting IL-2 secretion the promotion of T-cell proliferation is also inhibited.

In yet another embodiment, the present invention provides methods for inhibiting TNF-α secretion from a cell by contacting the cell with a composition that contains a concentrated inflammation modifier. TNF-α is an important proinflammatory mediator; it upregulates the expression of other cytokines, chemokines, adhesion molecules, proteases, and enzymes involved in prostaglandin, leukotriene, and nitric oxide production. By inhibiting TNF-α secretion, the inflammatory response is thereby decreased. For example, TNF-α activates inflammatory leukocytes to kill microbes and TNF-α is especially potent at activating neutrophils, but also affects eosinophils and mononuclear phagocytes.

A. Treatment of Skin and Mucosal Membrane Inflammation by Administering Selected Concentrated Inflammation Modifiers

The invention provides methods for treating inflammatory conditions and diseases of the skin and mucosal membranes. In some embodiments, the treatment involves administering a therapeutically effective amount of a concentrated inflammation modifier as described herein. Preferred embodiments involve administering an effective amount of an concentrated inflammation modifier obtained by fractionating petrolatum or lanolin or lanolin-related compounds. In particular, preferred anti-inflammatory compositions include one or more of Fractions 6-9 of lanolin; with Fraction 8 being most preferred. An effective amount is that amount which can reduce or eliminate the inflammation and/or symptoms of the inflammation. Because these selected concentrated inflammation modifiers are each effective anti-inflammatory agents alone, the compositions comprising the fraction can be essentially free of other anti-inflammatory agents, or can increase the potency of other anti-inflammatories.

The methods described herein find use in the treatment of a variety of skin disorders, including those associated with keratinocyte differentiation and proliferation, for example, psoriasis, atopic dermatitis, allergic dermatitis, eczematous or atopic dermatitis, acne vulgaris, comedones, polymorphs, nodulokystic acne, conglobata, senile acne, secondary acne such as solar acne, medicinal acne, irritant contact dermatitis or professional acne; other types of keratinization disorders, for example, ichthyoses, ichthyosiform conditions, Darier malady, palmoplantary keratodermies, leucoplasies and leucoplasiform conditions and lichen; other dermatologic disorders such as cutaneous T-cell lymphoma, blistery dermatoses and collagen maladies; and aging of the skin, be it photoinduced or not. Other specific skin and mucosal membrane inflammatory diseases and conditions that are amenable to treatment using the methods provided include, but are not limited to, irritant contact dermatitis, allergic contact dermatitis, T-cell and B-cell mediated skin disorders, and skin disorders that are caused by local inflammatory mediator release.

To modulate an inflammatory condition of the skin or mucosal membrane, a composition that contains one or more concentrated inflammation modifiers is administered to the skin or mucosal membrane in an amount effective to modulate the inflammatory condition. An effective amount can be determined by applying the compositions containing the anti-inflammatory agent to test animal models as described herein. Typically, the compositions that are useful in the claimed methods include one or more concentrated inflammation modifiers at a concentration of between 0.001% and 100%. Preferably the concentrated inflammation modifier concentration will be between about 0.15% and about 15%. The compositions can also include additional ingredients, as described below, but an additional anti-inflammatory agent is not generally required. For example, for acute diseases such as allergic and irritant contact dermatitis, the inflammatory reaction can be prevented by prophylactic application of the described concentrated inflammation modifiers, either alone or in combination with a penetration blocking agent.

In a preferred embodiment, the compositions that contain-one or more concentrated inflammation modifiers can contain an oil that is high in unsaturated fatty acids. Examples of such oils that are useful include, but are not limited to, safflower oil, almond oil, amyl butyrate, apricot kernel oil, avocado oil, camphor, castor oil, 1-carvone, coconut oil, corn oil, cotton seed oil, eugenol, menthol, oil of anise, oil of clove, orange oil, peanut oil, peppermint oil, rose oil, sesame oil, shark liver oil (squalene), soybean oil, sunflower oil, and walnut oil. The oils are typically present in the compositions at a concentration of between about 0.1% and about 10%, preferably between about 0.5% and about 5%.

Treatment can be repeated until the inflammation and associated symptoms are reduced or eliminated. Generally, application of the concentrated inflammation modifier compositions is repeated at intervals of about once or twice per day, although more or less frequent administration is advisable where so indicated by the severity and discomfort associated with the inflammatory condition.

B. Treatment of Sensitization, Inflammation, and Irritation Accompanying Transdermal Drug Delivery or the Delivery of Drugs Using an Electric Field

The present invention also provides methods for reducing or eliminating inflammation, sensitization, and irritation that can result from administration of pharmacological agents via transdermal dosage forms, including transdermal patches, iontophoresis, sonophoresis, electroporation, and the like. These methods involve administering a concentrated inflammation modifier to the area of the skin to which the transdermal dosage form is applied.

Transdermal dosage forms are advantageous for many applications because they provide for controlled delivery of a pharmacological agent to the skin or body. See TRANSDERMAL DRUG DELIVERY: DEVELOPMENTAL ISSUES AND RESEARCH INITIATIVES, Hadgraft and Guy (eds.), Marcel Dekker, Inc., (1989); CONTROLLED DRUG DELIVERY: FUNDAMENTALS AND APPLICATIONS, Robinson and Lee (eds.), Marcel Dekker Inc., (1987); and TRANSDERMAL DELIVERY OF DRUGS, Vols. 1-3, Kydonieus and Berner (eds.), CRC Press, (1987). Such dosage forms can be made by dissolving, dispersing or otherwise incorporating the pharmacological agent in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

However, it is well recognized that application of drug-delivering transdermal delivery systems to the skin can result in the development of an immediate or delayed-type contact sensitivity to the drug being delivered or even to components of the delivery system. In addition, a variety of other inflammatory/immune related side effects, including edema, erythema, urticaria, hyperpigmentation, sensitization, and the like have been reported with the use of transdermal drug delivery devices and topical drug delivery.

1. Transdermal Patch

The methods described herein are useful, with a variety of types of transdermal delivery methods. For example the described methods are useful for modulating inflammation caused by an inflammation-inducing pharmacological agent that is administered using an adhesive patch, or adhesive matrix patch, which can be prepared from a backing material and an adhesive, such as an acrylate adhesive. The concentrated inflammation modifiers, the pharmacological agent, and any enhancer, or combination of enhancers, can be formulated into the adhesive casting solution and allowed to mix thoroughly. The solution is cast directly onto the backing material and the casting solvent is evaporated in an oven, leaving an adhesive film. A release liner can be attached to complete the system. Examples of adhesive matrix-type transdermal patches are found in U.S. Pat. Nos. 5,302,395, Ebert et al.; 5,262,165, Govil et al.; 5,248,501, Parnell, F. W.; 5,232,702, Pfister et al.; 5,230,896, Yeh et al.; 5,227,169, Heiber, S., et al.; 5,212,199, Heiber et al.; 5,202,125, Ebert et al.; 5,173,302, Holmblad, et al.; 5,154,922, Govil, et al.; 5,139,786, Ferrini et al.; 5,122,383, Heiber et al.; 5,023,252, Hseih, D.; and 4,978,532, El-Rashidy, T. Each of these disclosures is incorporated herein by reference.

The methods and compositions are also useful for modulating inflammation that arises from the use of a polyurethane matrix patch to deliver the pharmacological agent. The layers of this patch comprise a backing, a polyurethane drug/enhancer matrix, a membrane, an adhesive, and a release liner. The polyurethane matrix is prepared using a room temperature curing polyurethane prepolymer. Addition of water, alcohol, and drug to the prepolymer results in the formation of a tacky firm elastomer that can be directly cast onto the backing material. A further embodiment of this invention will utilize a hydrogel matrix patch. Typically, the hydrogel matrix will comprise alcohol, water, drug, and several hydrophilic polymers. This hydrogel matrix can be incorporated into a transdermal patch between the backing and the adhesive layer. Examples of such patches were described in, for example, U.S. Pat. Nos. 5,324,521, Gertner, et al.; 5,306,503, Muller, W.; 5,302,395, Ebert, C. D.; 5,296,230, Chien, et al.; 5,286,491, Amkraut, et al.; 5,252,334, Chiang, et al.; 5,248,501, Parnell, F. W.; 5,230,896, Yeh, et al.; 5,227,169, Heiber et al.; 5,212,199, Heiber et al.; 5,202,125, Ebert, C. D.; 5,173,302, Bergstrom, et al.; 5,171,576, Amkraut, et al.; 5,139,786, Ferrini, et al.; 5,133,972, Ferrini, et al.; 5,122,383, Heiber, et al.; 5,120,546, Hansen, J. A.; 5,118,509, Amkraut, A.; 5,077,054, Amkraut et al.; 5,066,494, Becher, F.; 5,049,387, Amkraut, A.; 5,028,435, Katz, et al.; 5,023,252, Hseih, D.; 5,000,956, Shaw, J. E.; 4,911,916, Cleary, G. W.; 4,898,734, Mathiowitz, et al.; 4,883,669, Chien, et al.; 4,882,377, Sweet et al.; 4,840,796, Sweet et al.; 4,818,540, Chien et al.; 4,814,173, Song et al.; 4,806,341, Chien et al.; 4,789,547, Song et al.; 4,786,277, Powers et al.; 4,702,732, Powers et al.; 4,690,683, Chien et al.; and 4,627,429, Tsuk, A. G.; and 4,585,452, Sablotsky, S.

Another type of patch for which the described anti-inflammatory methods and compositions are useful is the liquid reservoir patch. This patch comprises an impermeable or semi-permeable, heat sealable backing material, a heat sealable membrane, an acrylate based pressure sensitive skin adhesive, and a siliconized release liner. The backing is heat sealed to the membrane to form a reservoir which can then be filled with a solution of the drug, enhancers, gelling agent, and other excipients. Such patches were described in, for example, U.S. Pat. Nos. 5,324,521, Gertner, et al.; 5,300,299, Sweet, et al.; 5,275,819, Amer, et al.; 5,246,705, Venkatraman, et al.; 5,242,111, Nakoneczny, et al.; 5,232,702, Pfister, et al.; 5,227,169, Heiber, et al.; 5,213,568, Lattin, et al.; 5,212,199, Heiber, et al.; 5,156,591, Gross, et al.; 5,122,383, Heiber, et al.; 5,108,710, Little, et al.; 5,013,552, Amer, et al.; 4,999,379, Fankhauser, P.; 4,810,499, Nuwayser, E. S.; 4,777,170, Heinrich, W. A.; and 4,687,481, Nuwayser, E.

Similarly, the methods and compositions are useful with foam matrix patches, which are similar in design and components to the liquid reservoir system, except that the gelled drug solution is constrained in a thin foam layer, typically a polyurethane. This foam layer is situated between the backing and the membrane which have been heat sealed at the periphery of the patch.

There are at least seven transdermal therapeutic systems presently on the market: scopolamine (motion sickness), nitroglycerin (angina), clonidine (hypertension), estradiol (hormone replacement), nicotine (smoking cessation), fentanyl (analgesic) and testosterone (hypogonadism). See, e.g., THE PHYSICIAN'S DESK REFERENCE (“PDR”), 48th Ed. (Medical Economic Data 1994), which is incorporated herein by reference. Among all the transdermal patch users, “irritation” is observed in approximately 15% to 20% of the population with various degree of severity. This number does not include those cases where the irritation and/or sensitization was so prominent (for example, transdermal delivery systems for β-blockers such as propranolol, antihistamines, etc.) that the overall development of these patches was suspended.

Irritation developed from either acute and especially chronic use of the transdermal patches could be a result of the vehicle, enhancer, adhesive, drug, or any combinations of these components. Both irritant and allergic contact dermatitis have been observed. For example, clonidine is known to cause allergic contact dermatitis. See, PDR, supra. The methods and concentrated inflammation modifiers described in this application can be used before, during, or subsequent to the application of a transdermal patch and can be incorporated in the same patch, or in a separate dosage form. Therefore, irritation due to the patch application can either be prevented by pretreatment and/or co-administration with the transdermal candidate, or be treated by the method and agents mentioned in this application following the use of transdermal patches to further improve the safety profile and compliance of any given transdermal product.

Methods of pretreatment include applying one or more of the compositions described above to the skin in the form of a topical preparation such as an ointment, gel or cream prior to the administration of the transdermal patch; for example, the compositions can be applied about 1-2 hours prior to administration of the patch. The methods can also prevent or reduce inflammation that is induced by a drug that causes inflammation as an undesirable side effect, as well as inflammation that is associated with the attachment of transdermal patches to the skin. The concentration of concentrated inflammation modifier present in the compositions used in these methods can range from about 0.015% to 100%. Preferably, the concentrated inflammation modifier will be present in the compositions at a concentration of between about 0.05% and 30%, and more preferably between about 1% and 10%. The compositions can include additional ingredients, including, but not limited to, safflower oil, avocado oil, cholesterol, glycerin, and the like.

The compositions of the invention can be applied to the skin prior to application of a transdermal dosage form, or after the transdermal dosage form has been removed. The anti-inflammatory compositions are applied to the area of the skin to which the dosage form was, or is to be, placed. A sufficient amount of the composition is applied to completely cover the surface area of the transdermal patch, generally about 20 cm². A dosage of concentrated inflammation modifier that is effective to prevent or reduce inflammation associated with transdermal delivery of a drug can easily be determined by using animal models such as those described herein. Transdermal patches are applied to the animal with or without a composition comprising the concentrated inflammation modifier in different amounts, and the extent to which inflammation is reduced compared to controls that do not receive a concentrated inflammation modifier is determined. Generally, the concentration of concentrated inflammation modifier present in the compositions used in these methods is as described above for pretreatment.

In another embodiment, the concentrated inflammation modifiers can be applied to the skin at the same time as the device or delivery system that is used to deliver the inflammation-inducing drug, or other excipient including adhesive polymers. For example, the concentrated inflammation modifiers can be incorporated into a transdermal patch, in combination with one or more different drugs. The concentrated inflammation modifiers can be present in the dosage form as a mixture with the drug being delivered, or can be present in a separate reservoir or layer. The concentrated inflammation modifiers can also be present in the adhesive layer of a transdermal dosage form. In addition, the concentrated inflammation modifiers of the present invention can be combined with known anti-inflammatory agents such as hydrocortisone, to generate combinations with enhanced activity.

2. Occlusion

The concentrated inflammation modifiers and methods of the invention are also useful for preventing or reducing inflammation and or maceration of the local tissue that can result from drug delivery by occlusion, which comprises the application of a hydrated dressing, optionally with a pharmacological agent and a sealing material overlaid on the outside, to the area of skin to be treated. Occlusion prevents loss of the drug from the skin, promotes skin hydration, and increases skin temperature. These actions have been shown to enhance the penetration of certain medications used in the treatment of psoriasis, leg ulcers, some dermatitis and ekratodermas. The concentrated inflammation modifiers described herein can be administered in conjunction with any number of different occlusive dressings, including those described below, to treat inflammatory conditions that arise as a result of the drug delivery.

A variety of synthetic occlusive dressings with variable occlusive properties are now available for use in promoting topical drug absorption and wound healing. Examples include hydrocolloids, such as hydroactive particles in a hydrophobic polymer (e.g., DuoDERM® Dressing, Convatec/Division, Squibb Co., Princeton, N.J.); hydrogels, such as water and polyethylene oxide reinforced with polyethylene film (e.g., Vigilon®, Bard Home Health Division, Berkeley Heights, N.J. and Spenco 2nd Skin Dressing®, Spenco Medical Inc., Ward, Tex.); polyethylene (e.g., Glad Cling Wrap®, Union Carbide Corp., Home and Automotive Products Division, Danbury, Conn.); polyurethane (e.g., Op-Site®, Smith & Nephew, Welwyn Garden City, Hertfordshire, England; Tegaderm®, 3M Company, Medical Surgical Division, St. Paul, Minn.; Bioclusive®, Johnson & Johnson Products, New Brunswick, N.J.; and Ensure®, Becton Dickinson Acute Care/Deseret, Sandy, Utah); polyvinylidine (e.g., Saran Wrap®, Dow Chemical Co., Indianapolis, Ind.); and siloxane or silicone dressings, including dimethylpolysiloxane polymer-elastomer bonded to a finely knit nylon fabric (e.g., Biobrane®, Woodroof, Santa Ana, Calif.). See, also, American Medical Association (1992) Drug Evaluations (Subscriptions), Section 1. The concentrated inflammation modifiers described herein can be used in conjunction with these dressings.

3. Iontophoresis

Similar methods are also useful for preventing or reducing inflammation that can result from delivery of drugs by iontophoresis and related methods, which involve the use of an electric field to administer a therapeutic agent. The term “electrotransport” is used to refer to methods and apparatus for transdermal delivery of therapeutic agents by means of an applied electromotive force to an electrolyte-containing reservoir. The particular therapeutic agent being delivered may be charged or uncharged, depending upon the particular method chosen.

The generation of pain and bums has been reported during electrotransport treatments. Erythema, a primary cutaneous reaction to irritant stimuli, has been frequently noted as a reaction to electrotransport but usually resolves without sequelae and is not necessarily associated with any permanent damage to the skin. Erythema associated with electrotransport can arise as a result of mild, non-specific irritation, for example, delivery of an irritant drug. The methods claimed herein provide means for controlling the sensitization, inflammation, and/or irritation accompanying transdermal drug delivery. The agents of the present invention may be used before, during or following the application of electrotransport to reduce irritation and sensitization resulting from such application. Such transdermal- and iontophoresis-related inflammation is described in, e.g., Hogan, et al., J. Am. Acad. Dermatol., 22:811-814 (1991); Holdiness, Contact Dermatitis, 20: 3-9 (1989); Ledger, Advanced Drug Delivery Reviews, 9: 289-307 (1992); and Lynch, et al., J. Control. Release, 6: 39-50 (1987).

Therefore, according to another aspect of the present invention, the inflammation, irritation, and/or sensitization which frequently occurs with transdermal or iontophoretic delivery of drugs, and in other topical products such as cosmetics, can be ameliorated by pre-, co-, or post-administration of a concentrated inflammation modifier as described herein. The agent or agents may be administered to the skin prophylactically, i.e., before the application of the iontophoretic current either topically or subcutaneously, or the agent or agents may be administered contemporaneously with the iontophoretic current, for example, by inclusion of the agent or agents with the reservoir of material to be delivered to the skin.

By way of terminology, when the therapeutic species being delivered is charged, for example potassium ion, calcium ion, or any charged atom or molecule, the process is referred to as iontophoresis. When the therapeutic species delivered is uncharged, it may be considered delivered by means of electro-osmosis techniques or other electrokinetic phenomenon such as electrohydrokinesis, electro-convection or electrically-induced osmosis. In general, these latter electrokinetic delivery processes of uncharged species into a tissue result from the migration of solvent, in which the uncharged species is dissolved, as a result of the application of electromotive force to the electrolyte reservoir. Of course during the process, some transport of charged species will take place as well.

In general, iontophoresis is an introduction, by means of electric current, of ions of soluble salts into the tissues of the body. More specifically, iontophoresis is a process and technique which involves the transfer of ionic (charged) species into a tissue (for example through the skin of a patient) by the passage of a electric current through an electrolyte solution containing ionic molecules to be delivered (or precursors for those ions), upon application of an appropriate electrode polarity. That is, ions are transferred into the tissue, from an electrolyte reservoir, by application of electromotive force to the electrolyte reservoir.

If the electrotransport method is iontophoresis, generally the active electrode includes the therapeutic species as a charged ion, or a precursor for the charged ion, and the transport occurs through application of the electromotive force to the charged therapeutic species. If other electrotransport phenomenon are involved, the therapeutic species will be delivered in an uncharged form, transfer being motivated, however, by electromotive force. For example, the applied current may induce movement of a non-therapeutic species, which carries with it water into the subject. The water may have dissolved therein the therapeutic species. Thus, electrotransport of the non-therapeutic charged species induces movement of the therapeutic but non-charged species.

Through iontophoresis, either positively charged drugs (medication) or negatively charged drugs (medication) can be readily transported through the skin and into the patient. This is done by setting up an appropriate potential between two electrode systems (anode and cathode) in electrical contact with the skin. If a positively charged drug is to be delivered through the skin, an appropriate electromotive force can be generated by orienting the positively charged drug species at a reservoir associated with the anode. Similarly, if the ion to be transferred across the skin is negatively charged, appropriate electromotive force can be generated by positioning the drug in a reservoir at the cathode. Of course, a single system can be utilized to transfer both positively charged and negatively charged drugs into a patient at a given time; and, more than one cathodic drug and/or more than one anodic drug may be delivered from a single system during a selected operation. For general discussions of iontophoresis, see, e.g., Tyle (1989) J. Pharm. Sci. 75:318; Burnette, Iontophoresis (Chapter 11) in Transdermal Drug Delivery, Hadgraft and Guy (eds.) Marcel Dekker, Inc.: New York, N.Y.; Phipps et al. (1988) Solid State Ionics 28-30:1778-1783; Phipps et al. (1989) J. Pharm. Sciences 78:365-369; and Chien et al. (1988) J. Controlled Release, 7:1-24, the full disclosures of which are incorporated herein by reference.

A wide variety of iontophoresis devices are presently known. See, e.g., Phipps et al., U.S. Pat. No. 4,744,788; Phipps et al., U.S. Pat. No. 4,747,819; Tapper et al., European Patent Application Publication No. 0318776; Jacobsen et al., European Patent Application Publication No. 0299631; Petelenz et al., U.S. Pat. No. 4,752,285; Sanderson et al., U.S. Pat. No. 4,722,726; Phipps et al., U.S. Pat. No. 5,125,894; and Parsi, U.S. Pat. No. 4,731,049, the full disclosures of each which are incorporated herein by reference.

In typical, conventional, electrotransport devices, for example iontophoresis devices, two electrodes are generally used. Both electrodes are disposed so as to be in intimate electrical contact with some portion (typically skin) of the subject (human or animal) typically by means of two remote electrolyte-containing reservoirs, between which current passes as it moves between the skin and the electrodes. One electrode, generally referred to herein as the “active” electrode, is the electrode from which the substance (medicament, drug precursor or drug) is delivered or driven into the body by application of the electromotive force. The other electrode, typically referred to as an “indifferent” or “ground” electrode, serves to close the electrical circuit through the body. In some instances both electrodes may be “active”, i.e. drugs may be delivered from both. Herein the term electrode, or variants thereof, when used in this context refers to an electrically conductive member, through which a current passes during operation.

A variety of electrode materials, ranging from platinum to silver-silver chloride, are available for these devices. The primary difference in these materials is not in their ability to generate an electric potential across the skin, but rather in certain nuances associated with their performance of this function. For example, platinum electrodes hydrolyze water, thus liberating hydrogen ions and subsequently, changes in pH. Obviously, changes in pH can influence the ionization state of therapeutic agents and their resulting rate of iontophoretic transport. Silver-silver chloride electrodes, on the other hand, do not hydrolyze water. However, these electrodes require the presence of chloride ion which may compete for current-induced transport.

Electrotransport devices generally require a reservoir as a source of the species (or a precursor of such species) which is to be moved or introduced into the body. The reservoir typically will comprise a pool of electrolyte solution, for example an aqueous electrolyte solution or a hydrophilic, electrolyte-containing, gel or gel matrix, semi-solid, foam, or absorbent material. Such drug reservoirs, when electrically connected to the anode or the cathode of an iontophoresis device, provide a source of one or more ionic species for electrotransport.

Generally, buffers will also be incorporated into the reservoir to maintain the reservoir environment at the same charge as the electrode. Typically, to minimize competition for the electric current, a buffer having the opposite charge to the drug will be employed. In some situations, for example, when the appropriate salt is used, the drug may act as its own buffer.

Other variables which may effect the rate of transport include drug concentration, buffer concentration, ionic strength, nonaqueous cosolvents, and any other constituents in the formulation. In general, to achieve the highest transport efficiency, the concentration of all ionic species, save the therapeutic agent itself, is minimized.

In conjunction with the patient's skin in electrical communication with the electrodes, the circuit is completed by connection of the two electrodes to a source of electrical energy as a direct current; for example, a battery or a source of appropriately modified alternating current. As an example, if the ionic substance to be driven to the body is positively charged, then the positive electrode (the anode) will be the active electrode and the negative electrode (the cathode) will serve to complete the circuit. If the ionic substance to be delivered is negatively charged, then the negative electrode (cathode) will be the active electrode and the positive electrode (anode) will be the indifferent electrode.

Chemical enhancers, vasodilators, and electroporation can also be utilized to alter the iontophoretic transport rate. For example, the co-application of oleic acid to the skin causes a large decrease in the skin impedance or resistance which is inversely related to permeability or transport. See Potts et al. (1992) Solid State Ionics 53-56:165-169. Thus, instead of the current passing primarily through the shunt pathways (e.g., the follicles and sweat ducts), the ions constituting the current can more uniformly permeate the lipid milieu of the stratum corneum at a lower current density. Thus, the epidermis, as well as the peripheral neurons surrounding the hair follicles and sweat ducts, will be able to experience the electrical stimulation.

In electroporation, which may have the same net effect as the use of chemical enhancers plus conventional iontophoresis, transient pores in the lipid structure of membranes, such as the stratum corneum, are created by the applied electric field. Such a technique has been used to introduce DNA into various cells. See Chang et al. (1992) HANDBOOK OF ELECTROPORATION AND ELECTROFUSION Academic Press: New York. Generally, electroporation involves the application of infrequent, short (about 1 millisecond), high voltage (5-300 volts) electric pulses.

4. Combinations

The described methods for reducing or eliminating inflammation are also useful when combinations of the various techniques described herein, i.e., electrotransport, sonophoresis, pharmacological intervention, and occlusion, are utilized. For example, pharmacological agents can be administered “actively” through the use of iontophoresis, or sonophoresis, optionally with stratum corneum lipid perturbants, or “passively”, for example via the topical application of pharmacological agents, alone or with stratum corneum lipid perturbants. A further embodiment will combine iontophoresis with occlusion. Other embodiments will provide for the combination of occlusion and pharmacological agents. For example, microparticle encapsulated drugs can be suspended in the hydrophilic gel of an occlusive dressing. In addition, for the treatment of animals, pharmacological intervention frequently will be combined with occlusion. Still other embodiments will include the combination of various pharmacological agents. Still further embodiments of the invention include combinations of pharmacological agents which are administered by a combination of methods, described above. Alternatively, the patient may receive concurrent treatments with the various therapies. For example, an occlusive dressing containing a pharmacological agent may be applied to the affected area. Alternatively, a pharmacological agent may be delivered iontophoretically. In any of these situations, the compositions that contain concentrated inflammation modifiers can be used to reduce or eliminate inflammation that results as an undesirable side effect of the drug treatment.

C. Treatment of Wounds

The major cause of chronic wounds is poor local blood circulation (ischemia) which leads to tissue infarction followed by secondary infection. A partial list of the causes of chronic ulcers of the skin includes circulatory disturbances, such as varicose veins and obliterative arterial disease, extensive injury from frostbite or burns, trophic changes accompanying many neurologic disorders, bedsores or decubiti, systemic diseases such as sickle cell disease, neoplasms, diabetes and various infections. See PROGRESS IN CLINICAL AND BIOLOGICAL RESEARCH, Vol. 365, “Clinical and Experimental Approaches to Dermal and Epidermal Repair: Normal and Chronic Wounds”, Barbul et al. (eds.), Wiley-Liss, Inc., (1991). No matter what the underlying cause, secondary infection is very likely to occur and to interfere with healing, complicate grafting or other restorative procedures, or produce extension of the process. Since the concentration inflammation modifiers don not act in the same manner as glucocorticoids, they do not vasorestrict and thus decrease blood flow.

Current therapies for chronic wounds include debridement to remove the necrotic and possibly infected tissue and subsequently, promoting wound repair. Another therapeutic regimen which is presently under clinical development is skin or epidermal allografts. These treatment regimens involve major surgical procedures and significant medical costs and health risks.

The methods described herein provide a low-cost, non-surgical procedure for the treatment of wounds. Specifically, the methods provide a means for modulating the healing of a wound by administering a concentrated inflammation modifier as described herein. Wound conditions for which these methods are applicable include, but are not limited to, preventing maceration at peri-ulcer or peri-stomal skin, after facial peels or dermabration, epidermal blistering diseases, toxic epidermal necrolysis, erythema multiforme, superficial thermal bums, intertrigo, and premature infant skin. The methods are effective for both reducing the progression of wound development and for enhancing wound healing. The formulations useful for these methods are as described herein. Treatment of a wound with the compositions can begin immediately after the wound is incurred, and can continue until the wound is completely healed.

D. Treatment of Inflammation Associated With Cosmetics or Skin Care Products

The compositions and methods of the invention can also be used to alleviate skin sensitization, irritation or inflammation associated with cosmetics or skin care products. The concentrated inflammation modifiers described herein can be used before, during or in response to skin sensitization, irritation or inflammation associated with cosmetic, cosmeceutical, dermatological or other dosage forms.

Typical skin care products and cosmetics that can cause adverse skin reactions such as sensitization, irritation or inflammation include depilatories such as described in U.S. Pat. Nos. 5,271,942 and 5,296,472; hair color treatments such as described in U.S. Pat. No. 5,318,776; antiperspirants such as disclosed in U.S. Patent No. 5,298,236; fragrances, such as discussed in U.S. Pat. No. 5,297,732 and 5,278,141. Each of these patent disclosures is incorporated herein by reference. Other cosmetics include, e.g., creams, lotions, sunscreens, make-up preparations, face powders, lipsticks, mascaras, eye shadows, nail products, hair preparations, bath products, shaving preparations, soaps, and toothpastes. These compositions are formed using methods well known in the chemical arts see, e.g., THE KIRK-OTHMER ENCYCLOPEDIA OF CHEMICAL TECHNOLOGY, 3rd Ed., Vol. 7 (Wiley Interscience 1979), which is incorporated herein by reference.

E. Prevention and Treatment of Skin Aging

The compositions and methods of the present invention can also be used to prevent the development and also treat symptoms and signs associated with skin aging. The concentrated inflammation modifiers described herein can be used before exposure to environmental factors which are known to accelerate skin aging, e.g. ultraviolet light irradiation, or it can be applied after exposure to minimize the damage caused by the exposure. Furthermore, by stimulating the growth of dermal fibroblasts, the concentrated inflammation modifiers can also reverse the paucity of dermal cells observed in aged skin and provide a means to reverse aged skin.

As such, the present invention provides a method for preventing skin aging, the method including: applying a concentrated inflammation modifier to the skin thereby preventing skin aging. In certain aspects, the concentrated inflammation modifiers can be combined with other active agents. Typical agents include, but are not limited to, retinoic acid, retinol, and alpha-hydroxy acids in a lotion or a cream.

### EXAMPLES

The following examples are offered to illustrate, but not to limit the present invention.

### Example 1

### Process to Isolate the Active Fractions from Natural Sources

This Example describes a procedure for isolating and enriching concentrated inflammation modifiers of the invention from natural product mixtures. Fractions from one natural product mixture, lanolin, were isolated based on this procedure. Initial studies to examine the separation of lanolin components were carried out using one-dimensional and two-dimensional thin-layer chromatography (TLC). The results were used to select conditions for flash column chromatography separation of 10 and 20 grams of U.S.P. lanolin. Fractions were analyzed using TLC to assure that fractions contained a minimal amount of overlap of the same components.

The method was used to fractionate lanolin into 9 fractions. The first six fractions constitute approximately 93% of the weight of lanolin applied to the columns. Fractions 1 and 2 elute in very low polarity solvent systems and may be difficult to fractionate further. TLC data indicates that fractions 3 through 9 can be fractionated further using silica gel-based chromatography.

Initial Screening Using Thin-Layer Chromatography

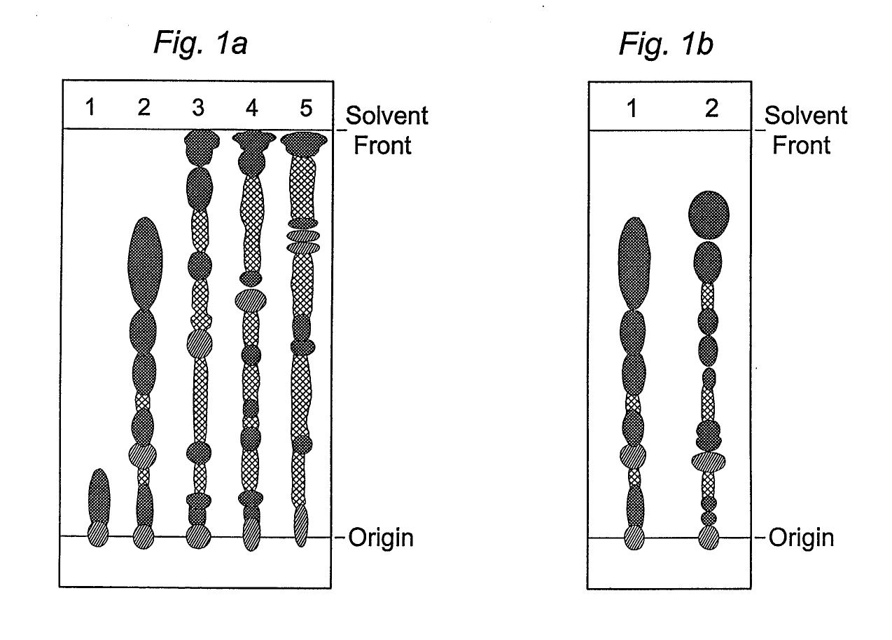

As a first step for developing a method to fractionate U.S.P. Lanolin, we explored several solvent systems using thin-layer chromatography. We focused on two-component solvent systems that had a very low polarity solvent (hexanes, carbon tetrachloride, benzene) mixed with a slightly polar agent (ethyl acetate, diethyl ether). Materials were visualized by spraying the TLC with sulfuric acid followed by flame-charring. FIG. 1 shows examples of TLC results. As shown in FIG. 1(a), a 2.5% ethyl acetate (EtOAc) in hexanes solvent system eluted a major portion of the lanolin above an R_{f} of 0.5. A second series of hexanes/EtOAc mixtures (99.4:0.6; 98.8:1.2; 98.2:1.8; 97.5:2.5) was used to evaluate the potential for using a gradient to fractionate lanolin. As shown in FIG. 1(b), an EtOAc/carbon tetrachloride (CCl₄) solvent system (98:2) gave slightly better resolution of the compounds. We decided to use a system based on carbon tetrachloride for our first attempt at lanolin fractionation.

Crude Fractionation Of Lanolin Using Column Chromatography

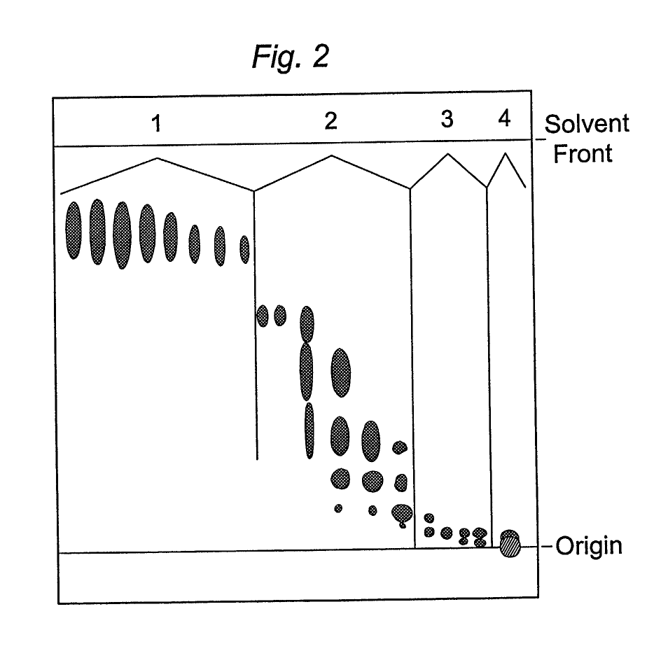

U.S.P. lanolin was then fractionated into 3 to 5 fractions using flash chromatography. These crude fractions would be subjected to further fractionation by using either a second flash column or a recycling high-performance liquid chromatography system. U.S.P. Lanolin (10.0 g; RITA Corporation Lot # 2385) was dissolved in a minimal amount of CCl₄ (˜30 mL) and applied to a bed of silica gel (200 mL, EM Science Kieselgel 60, 230-400 mesh ASTM) and eluted with CCl₄ (400 mL), CCl₄/EtOAc (400 mL; 99:1), CCl₄/EtOAc (400 mL, 98:2), and CCl₄/EtOAc (400 mL, 95:5). Fractions (˜70 mL) were collected analyzed by TLC using 2.5% EtOAc in hexanes as the eluant, shown in FIG. 2. Fractions 1 through 7 were combined and evaporated to give 4.0 grams (40%) of recovered material. Fractions 8 through 13 were combined and evaporated to give 3.4 grams (34%) of material. Fractions 14 through 17 were combined and evaporated to give 0.5 grams (5%) of material. Ethyl acetate (250 mL) was used to flush the column and the eluted material was collected as a single fraction. Evaporation gave 2.0 grams (20%) of material. Elution with EtOAc/methanol (300 mL; 90:10) resulted in the recovery of less than 100 mg of baseline material. The overall recovery of lanolin from the column was close to 100%.

Because fraction 1 from the first column accounts for approximately 40% of the mass of the lanolin applied to the column, it was necessary to find chromatography systems that could be used to further fractionate the material. A TLC system of diethyl ether/hexanes (2:98) indicated that the fraction could be separated into two discreet subfractions. Attempts to separate fraction 1 using a Japan Analytical Industry LC-908-G30 recycling HPLC equipped with a Waters PrepPak radial compression silica gel column (40∞300 mm) failed.

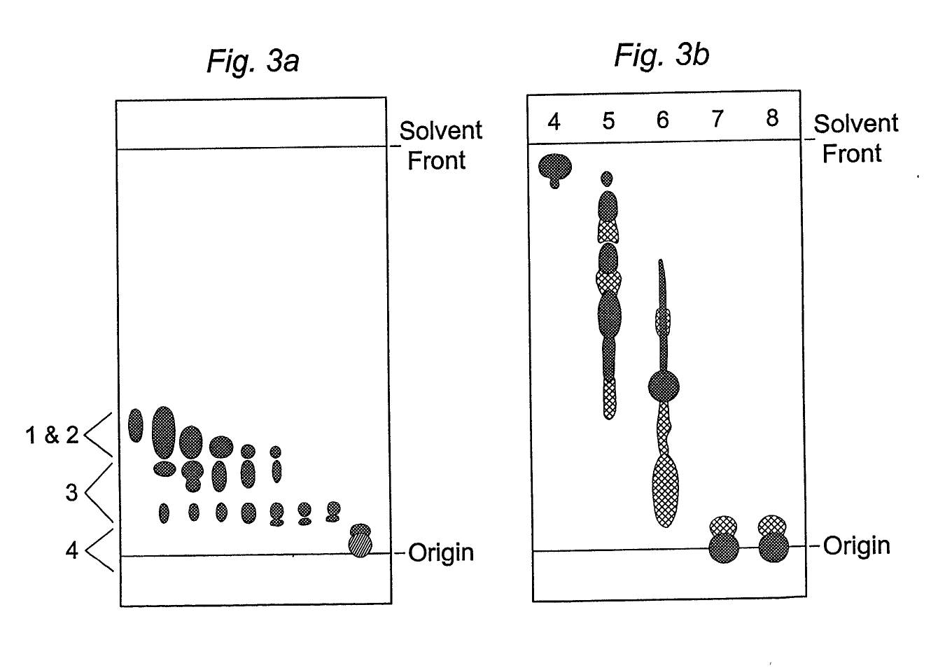

A larger scale fractionation of lanolin was conducted using a hexane/ethyl acetate gradient. Although CCl₄-based solvent systems showed better resolution on TLC, the toxicity of CCl₄ makes it a poor choice for scale-up. U.S.P. Lanolin (20.0 g; RITA Corporation Lot # 2385) was dissolved in a minimal amount of hexanes (˜100 mL), applied to a bed of silica gel (350 mL; EM Science Kieselgel 60, 230-400 mesh ASTM), and then eluted with hexanes (1L), hexanes/EtOAc (1L, 99:1), hexanes/EtOAc (1L, 98:2), hexanes/EtOAc (1L, 97:3), hexanes/EtOAc (1L, 95:5), hexanes/EtOAc (1L, 90:10), hexanes/EtOAc (1L, 75:25), EtOAc (1L), methanol/EtOAc (500 mL; 10:90). Fractions (˜200 mL) were collected and analyzed by TLC. As shown in FIG. 3, the hexanes/EtOAc column did not fractionate lanolin as well as the CCl₄/EtOAc column. The first 8 fractions containing material were combined and evaporated to give ˜11.5 g of material. Subsequent fractions were combined based on TLC data and evaporated to give lanolin fractions 4 through 9.

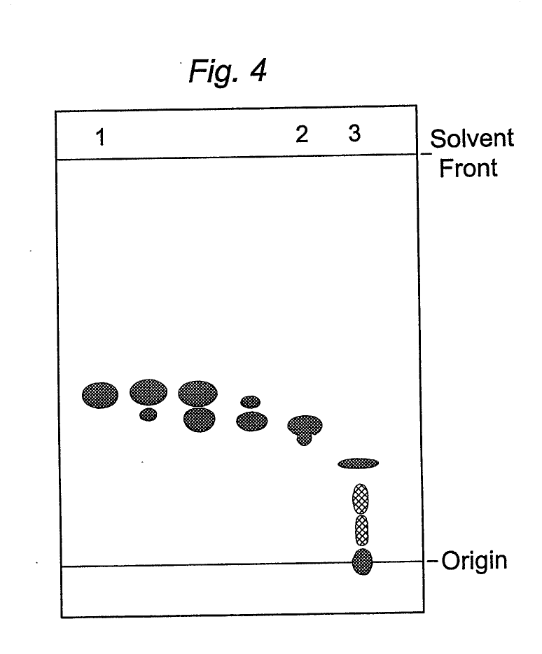

During this time, we discovered that hexanes/methylene chloride (CH₂Cl₂) (95:5) clearly separated fraction 1 from the first column into three distinct spots on TLC. Fraction 1 of column 2 was dissolved in hexanes (˜50 mL), applied to a bed of silica gel (200 mL; EM Science Kieselgel 60, 230-400 mesh ASTM, and then eluted with hexanes (250 mL), hexanes/CH₂Cl₂ (1 L, 95:5), hexanes/CH₂Cl₂ (500 mL, 90:10), and EtOAc (300 mL). As shown in FIG. 4, the elution gave fractions that contained only the highest moving spot (fraction 1), the lower two spots (fraction 2), and mixtures of the two. Ethyl acetate was used to flush the column of the lower R_{f} materials—these were combined and kept as fraction 3.

### Conclusions

We successfully fractionated lanolin into 9 fractions. As shown above in FIG. 3 and FIG. 4, fractions 1 through 6 have minimal overlaps in constituents. Based on the recoveries from the first column, fractions 1 and 2 represent approximately 40% of the material weight of the lanolin used. TLC suggests that fraction 1 is larger than 2 and may represent 25-30% of lanolin. Because of their mobility in nonpolar solvent systems further fractionation of fractions 1 and 2 using standard silica gel columns will be challenging. We estimate that fractions 3, 4, 5, and 6 constitute approximately 18%, 8%, 20%, and 7% of lanolin respectively. Each of these fractions contain multiple compounds that should be separable using flash silica gel chromatography. Fractions 7 through 9 represent a combined 7% weight of lanolin. These fractions were collected based on their resistance of elution from the column and may contain overlaps of components.

The 9 fractions were dissolved in diethyl ether, transferred to vials, and the ether removed under argon with mild heating (hot water bath). Some residual ether remained trapped within the fractions. Table 1 summarizes the amounts of materials obtained along with a physical description of the materials.

TABLE 1
LANOLIN FRACTIONATION

| Fraction | Reference Number | Mass | Physical Description |
|---|---|---|---|
| 1 | 10400-13-01 | 620 mg | white wax |
| 2 | 10400-13-02 | 710 mg | clear oil |
| 3 | 10400-13-03 | 1570 mg | white wax |
| 4 | 10400-13-04 | 1520 mg | creamy light yellow wax |
| 5 | 10400-13-05 | 1960 mg | creamy yellow wax |
| 6 | 10400-13-06 | 1330 mg | medium yellow wax |
| 7 | 10400-13-07 | 795 mg | deep yellow wax/oil |
| 8 | 10400-13-08 | 295 mg | yellow viscous oil |
| 9 | 10400-13-09 | 325 mg | yellow viscous oil/wax |

Using methylene chloride/hexanes and EtOAc/hexanes solvent systems indicates that the scale-up fractionation of lanolin is feasible. In addition to chromatographic techniques, one can further separate constituents by means of fractional distillation (Kugelrohr).

### Example 2

### Fractionation of Lanolin-Related Compounds

This Example describes the use of a chromatographic method to fractionate two other sources of materials, lanolin oil and avocadin. These were found to contain similar fractions to those obtained for lanolin.

Thin-Layer Chromatography

Two-dimensional thin-layer chromatography (2-D TLC) on a silica gel chromatography plate was performed to examine the separation of the materials derived from several natural sources. The plates were first eluted with methylene chloride in hexanes (1:4), and then eluted orthogonally with ethyl acetate in hexanes (1:19). The plates were visualized by sulfuric acid spray followed by charring. Numbered clusters correspond to the original fractions obtained for the first fractionation of lanolin.

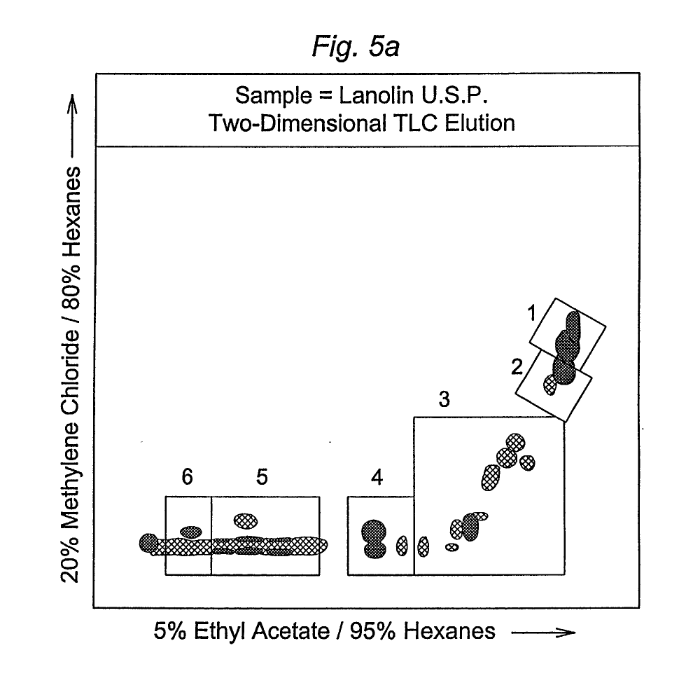

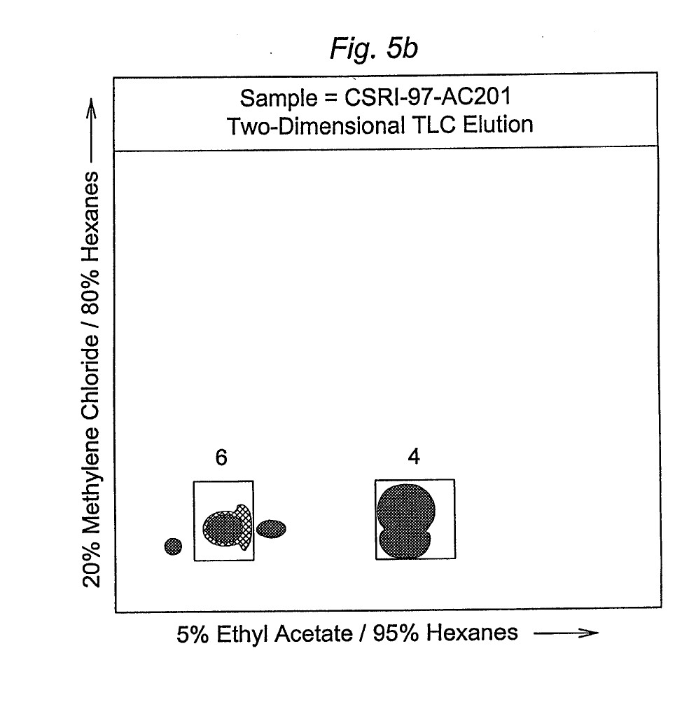

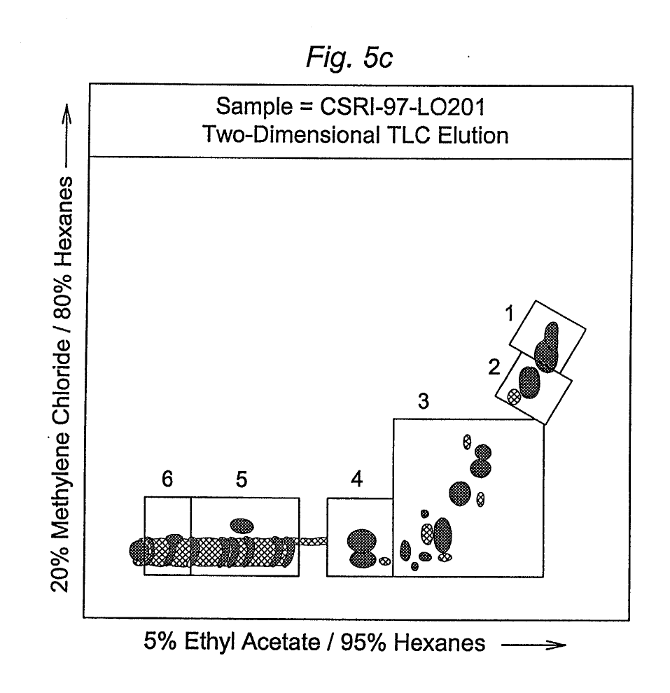

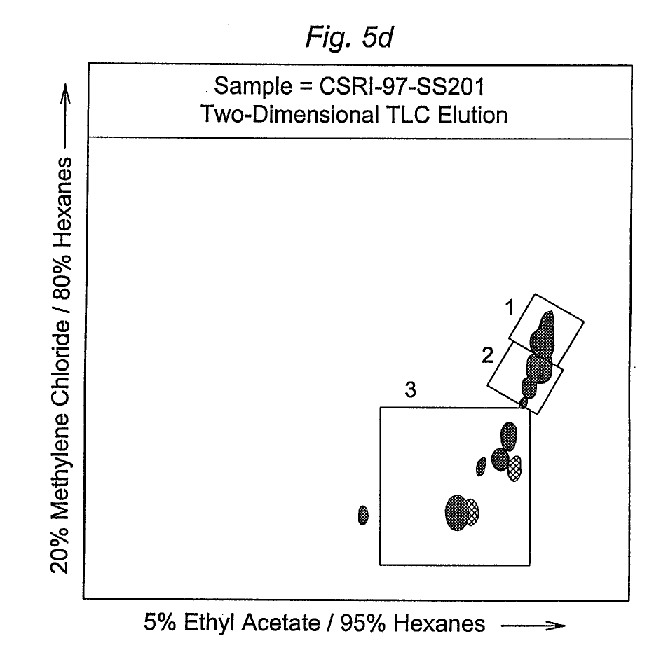

FIG. 5 shows that lanolin, lanolin oil (LO-201) and avocadin (AC-201), U.S.P. lanolin and lanolin oil (LO201) appear very similar in overall content. Avocadin (AC201), a preferred lipid material derived from avocado oil, contains two clusters of compounds, whereas super sterol ester (SS201) contains material found in the first three fractions of lanolin.

Material fractionation using column chromatography

The use of dichloromethane in hexanes as a flash silica gel chromatography eluant was found to provide the cleanest separation of components of lanolin and related compounds. It was apparent from the 2-D TLC study that this approach would be useful for lanolin oil (LO201) and super sterol ester (SS201), but would not be necessary for avocadin (AC201) because it consists mainly of fractions 1 through 3. A Merck 60 Flash Grade silica gel column (approximately 240-400 particle size) was used for the chromatographic separation.

Table 2 lists the amounts of the different eluants used for the chromatography of 30 grams of material on a 600 mL bed of flash chromatography grade silica gel. The columns were packed using 100% hexanes and the materials loaded using a minimum amount of hexanes (100 to 200 mL). The column was eluted with dichloromethane in hexanes (2.5:97.5) until the material corresponding from fraction 1 had come off the column or was being contaminated by material corresponding to fraction 2. The eluant was changed to dichloromethane in hexanes (5:95) and later to This was followed a step-gradient elution with ethyl acetate in hexanes to recover fractions 3 through 7. Elution with 100% ethyl acetate followed by methanol in ethyl acetate (10:90) led to the recovery of fractions 8 and 9. Eluted materials were collected in 200 mL fractions. These were evaluated by TLC and combined to give nine fractions that corresponded to the previously described TLC fractionation of lanolin.

TABLE 2
ELUTION PROTOCOL FOR THE FRACTIONATION
OF LANOLIN-RELATED MATERIALS

| | Approximate Solvent Amounts | | |
|---|---|---|---|
| | Used For Separation of: | | |
| Eluant | U.S.P. Lanolin | LO201 | SS201 |
| Dichloromethane/Hexanes (2.5:97.5) | 3.5 L | 3.5 L | 6 L |
| Dichloromethane/Hexanes (5:95) | 3 L | 3 L | 4 L |
| Dichloromethane/Hexanes (10:90) | 2.5 L | 2.5 L | 4 L |
| Ethyl acetate/hexanes (2:98) | 1.5 L | 1.5 L | |
| Ethyl acetate/hexanes (3:97) | 1.5 L | 1.5 L | |
| Ethyl acetate/hexanes (5:95) | 1.5 L | 1.5 L | 1.5 L |
| Ethyl acetate/hexanes (10:90) | 1.5 L | 1.5 L | |
| Ethyl acetate/hexanes (25:75)) | 1.5 L | 1.5 L | |
| Ethyl acetate | 1.5 L | 1.5 L | |
| Methanol/Ethyl acetate (10:90) | 1.5 L | 1.5 L | |

Using this protocol, U.S.P. lanolin was separated into nine fractions. Fraction 3, which contained a significant amount of the main component of fraction 4, was subjected to further chromatography. The material was dissolved in a minimal amount of hexanes, loaded onto an 80 mL bed of flash chromatography grade silica gel, and eluted with ethyl acetate in hexanes (2:98). Fractions containing the desired materials were combined and evaporated. Comparison with the previous fractionation of lanolin using TLC indicated the two sets of fractionated materials were very similar in nature.

Lanolin oil (LO201) was separated into nine fractions using the protocol listed in Table 2. Unlike what was observed for lanolin, no further purification of any of the fractions was required. The components observed in LO201 fractions are very similar to those observed for lanolin. Similar to lanolin, approximately 60% of the material is contained in the first three fractions.

Because super sterol ester (SS201) contains components that correspond to only the first three components of lanolin, the full protocol was not required to recover the materials from the column. After fractions 1 and 2 of lanolin oil (LO201) were eluted using the dichloromethane in hexanes gradient, ethyl acetate in hexanes (5:95) was used to quickly elute fraction 3.

Avocadin (AC201) was separated by dissolving 10 grams of the material in a minimal amount of dichloromethane and loading the material on a 150 mL bed of flash chromatography grade silica. Fraction 1, which accounts for over 90% of the material, was obtained by elution with additional dichloromethane. Fraction 2 was obtained by subsequent elution with 100% ethyl acetate.

Separated materials were evaporated under vacuum. The resulting compounds were dissolved in a minimal amount of dichloromethane and transferred to bottles and the solvent removed first by placing the material in an argon stream and residual solvents removed under vacuum (>10 microns). Complete removal of solvent can be complicated by the material either foaming or bumping out of the packaging. Thus, some solvent may have remained trapped in the samples.

Table 3 through Table 6 list the results of the fractionations of the various samples. Also included is a brief physical description and the amounts of materials obtained.

TABLE 3
LANOLIN FRACTIONATION

| Fraction | Reference Number | Mass | Physical Description |
|---|---|---|---|
| 1 | 10400-23-01 | 3.2 g | white wax |
| 2 | 10400-23-02 | 0.9 g | clear oil/wax |
| 3 | 10400-23-03 | 4.6 g | white wax |
| 4 | 10400-23-04 | 2.1 g | creamy light yellow wax |
| 5 | 10400-23-05 | 4.1 g | creamy yellow wax |
| 6 | 10400-23-06 | 1.4 g | medium yellow wax |
| 7 | 10400-23-07 | 1.8 g | deep yellow wax/oil |
| 8 | 10400-23-08 | 1.4 g | yellow viscous oil |
| 9 | 10400-23-09 | 0.4 g | yellow viscous oil/wax |

TABLE 4
LANOLIN OIL (LO201) FRACTIONATION

| | | | Equivalent | |
|---|---|---|---|---|
| Fraction | Ref. Number | Mass | Lanolin Fraction | Physical Description |
| 1 | 10400-25-01 | 3.1 g | 1 | white wax |
| 2 | 10400-25-02 | 1.6 g | 2 | clear oil |
| 3 | 10400-25-03 | 4.3 g | 3 | clear oil |
| 4 | 10400-25-04 | 1.5 g | 4 | creamy light yellow |
| | | | | wax |
| 5 | 10400-25-05 | 3.5 g | 5 | light yellow oil/wax |
| 6 | 10400-25-06 | 1.6 g | 6 | light yellow wax |
| 7 | 10400-25-07 | 1.6 g | 7 | yellow oil |
| 8 | 10400-25-08 | 2.5 g | 8 | yellow oil |
| 9 | 10400-25-09 | 0.8 g | 9 | yellow oil |

TABLE 5
SUPER STEROL ESTER (SS201) FRACTIONATION

| | | | Equivalent | |
|---|---|---|---|---|
| Fraction | Ref. Number | Mass | Lanolin Fraction | Physical Description |
| 1 | 10400-27-01 | 10.2 g | 1 | white wax |
| 2 | 10400-27-02 | 5.8 g | 2 | clear oil |
| 3 | 10400-27-03 | 6.7 g | 3 | white wax |

TABLE 6
AVOCADIN (AC201) FRACTIONATION

| | | | Equivalent | |
|---|---|---|---|---|
| Fraction | Ref. Number | Mass | Lanolin Fraction | Physical Description |
| 1 | 10400-29-01 | 5.2 g | 4 | pale yellow oil/wax |
| 2 | 10400-29-02 | 0.6 g | 6 | white solid |

Comparison of Fractions

Preliminary structural identification of these fractions was conducted, mainly via NMR spectroscopy technique. Selected information regarding the fractions obtained is presented in Table 7.

TABLE 7
Analysis of Fractions

| | Fraction | No. of UV absorbing | |
|---|---|---|---|
| Source | No. | components | Sterols apparent in NMR |
| U.S.P. Lanolin | 1 | 0 | cholesterol esters (1 or 2) |
| | | | signature peaks at δ 5.38 (bd), |
| | | | 4.61 (m), 1.03 (s), 0.92 (d), |
| | | | 0.84 (d), 0.68 (s) |
| Lanolin oil (LO201) | 1 | 0 | cholesterol esters (1 or 2) |
| | | | signature peaks at δ 5.38 (bd), |
| | | | 4.61 (m), 1.03 (s), 0.92 (d), |
| | | | 0.84 (d), 0.68 (s) |
| Super Sterol Ester | 1 | 0 | cholesterol esters (1 or 2) |
| (SS201) | | | signature peaks at δ 5.38 (bd), |
| | | | 4.61 (m), 1.03 (s), 0.92 (d), |
| | | | 0.84 (d), 0.68 (s) |
| U.S.P. Lanolin | 2 | 1 (very weak) | Small amount of cholesterol |
| | | | esters (1 or 2) signature peaks |
| | | | at δ 5.38 (bd), 4.61 (m), 1.03 |
| | | | (s), 0.92 (d), 0.84 (d), 0.68 (s). |
| | | | Another sterol appears to be |
| | | | present based on methyl |
| | | | singlets at δ 1.01 and 0.69. |
| Lanolin oil (LO201) | 2 | 0 | Small amount of cholesterol |
| | | | esters (1 or 2) signature peaks |
| | | | at δ 5.38 (bd), 4.61 (m), 1.03 |
| | | | (s), 0.92 (d), 0.84 (d), 0.68 (s). |
| | | | Another sterol appears to be |
| | | | present based on methyl |
| | | | singlets at δ 1.01 and 0.69. |
| Super Sterol Ester | 2 | 0 | Small amount of cholesterol |
| (SS201) | | | esters (1 or 2) signature peaks |
| | | | at δ 5.38 (bd), 4.61 (m), 1.03 |
| | | | (s), 0.92 (d), 0.84 (d), 0.68 (s). |
| | | | Another steroid appears to be |
| | | | present based on methyl |
| | | | singlets at δ 1.01 and 0.69. |
| U.S.P. Lanolin | 3 | 0 | none discernible |
| Lanolin oil (LO201) | 3 | 2 (minor) | none discernible |
| Super Sterol Ester | 3 | 0 | none discernible |
| (SS201) | | | |
| U.S.P. Lanolin | 4 | 2 (moderate/weak) | lanosterol ? |
| Lanolin oil (LO201) | 4 | 2 (moderate/weak) | lanosterol ? |
| Avocadin (AC201) | 1 | 0 | none discernible |

### Example 3

A glass column was packed with 30 kilograms of ICN 60 Å, 63-200 mesh silica. The column elution profile (see, Table 8) is the same as set forth in Example 2 with the exception of the following. The same mobile phase composition was used for fraction 5A as was used for fraction 4A. For fraction 10A, a new mobile phase composition of 50/50 hexane/ethyl acetate was used, for fraction 12A, 100% methanol was used in place of 90/10 ethyl acetate/methanol.

TABLE 8
ELUTION PROFILE FOR THE FRACTIONATION
OF U.S.P. LANOLIN

| | | | | Elution |
|---|---|---|---|---|
| | | Mobile Phase | | Volume |
| Fraction | Lot # | Composition | Percent | (Liters) |
| 1A | P124-93-1 | Hexane/methylene | 98.2 | 361 |
| | | chloride | | |
| 2A | P124-94-1 | Hexane/methylene | 95/5 | 323 |
| | | chloride | | |
| 3A | P124-95-1 | Hexane/methylene | 90/10 | 247 |
| | | chloride | | |
| 4A | P124-96-2 | Hexane/ethyl acetate | 98/2 | 152 |
| 5A | P124-97-1 | Hexane/ethyl acetate | 98/2 | 152 |
| 6A | P124-98-1 | Hexane/ethyl acetate | 97/3 | 152 |
| 7A | P124-99-1 | Hexane/ethyl acetate | 95/5 | 152 |
| 8A | P124-100-1 | Hexane/ethyl acetate | 90/10 | 152 |
| 9A | P124-101-2 | Hexane/ethyl acetate | 75/25 | 152 |
| 10A | P124-102-1/2 | Hexane/ethyl acetate | 50/50 | 152 |
| 11A | P124-103-1 | Ethyl acetate | 100 | 152 |
| 12A | P124-104-1 | Methanol | 100 | 152 |

Using this method, lanolin was separated into twelve fractions. Each fraction was concentrated in a 120-liter QVF under atmospheric conditions at a temperature of 70° C. to approximately 4 liters. The concentrate was transferred to a one liter round bottom flask over time and stripped to dryness under moderate vacuum at a temperature of 70° C. The four fractions of interest (8A, 9A, 10A, 11A) were placed under high vacuum and heated to 70° C. for three hours.

Table 9 lists the characteristics of the 12 fractions, Including the mass recovered for each fraction, the equivalent Lanolin fraction from example 2, and a physical description.

TABLE 9
THE FRACTIONATION OF U.S.P. LANOLIN

| | | | Equivalent | |
|---|---|---|---|---|
| | | | Fraction | |
| | | | from | Physical |
| Fraction | Lot # | Mass (g) | Example 2 | Description |
| 1A | P124-93-1 | 126.77 | *NC | White wax |
| 2A | P124-94-1 | 287.88 | NC | White wax |
| 3A | P124-95-1 | 460.36 | NC | White wax |
| 4A | P124-96-2 | 84.78 | NC | White wax |
| 5A | P124-97-1 | 558.38 | 3 | Yellow |
| | | | | wax |
| 5A | P124-97-2 | 217.95 | 3 | Yellow |
| | | | | wax |
| 6A | P124-98-1 | 197.24 | 4 | Yellow |
| | | | | wax |
| 7A | P124-99-1 | 187.04 | 5 | Yellow |
| | | | | wax |
| 8A | P124-100-1 | 264.85 | 6 | Yellow |
| | | | | wax |
| 9A | P124-101-2 | 294.09 | 7 | Yellow |
| | | | | wax |
| 10A | P124-102-1 | 59.22 | 8 | Dark |
| | | | | Yellow wax |
| 10A | P124-102-2 | 37.01 | 8 | Dark |
| | | | | yellow wax |
| 11A | P124-103-1 | 28.78 | 9 | Dark |
| | | | | yellow wax |
| 12A | P124-104-1 | 42.80 | NC | Dark |
| | | | | yellow wax |
| | | 2.85 kg | | |

*NC: No correlation by TLC against internal standard

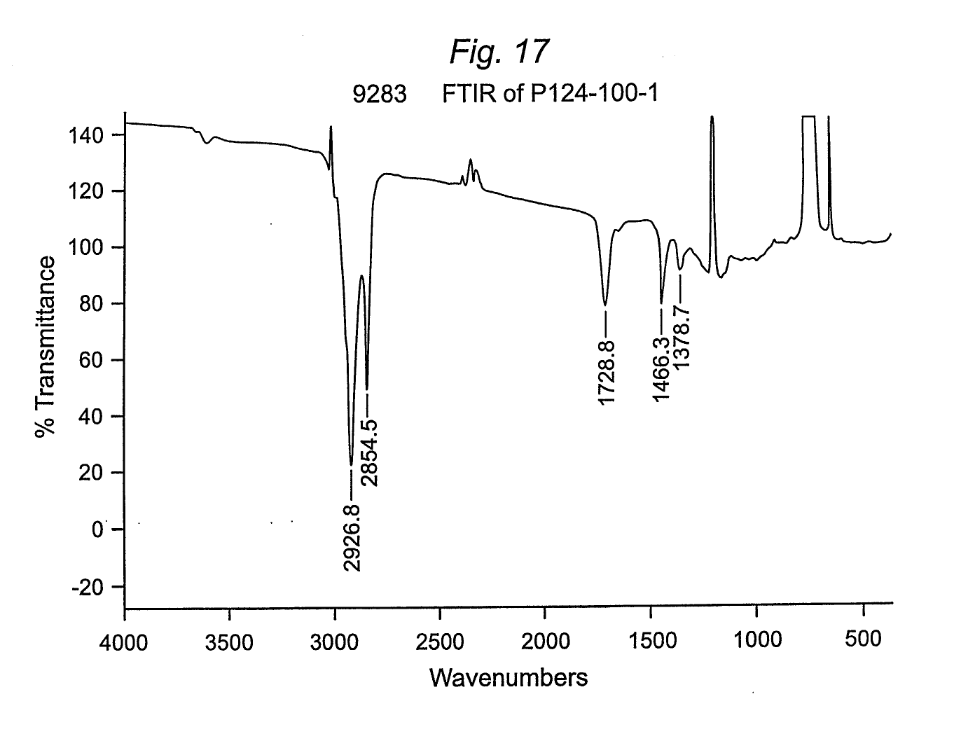

In addition to the physical descriptions of the fractions above, FIGS. 17-21 set forth Fourier infrared spectra obtained using a Digilab FTS-45 spectrometer of fractions 8A-12A, respectively. The IR spectra of these samples were obtained as a solution of 100 mg of sample in 2 mL of chloroform using a 0.050 mm path KBr cell, at 4 wave number resolution with chloroform being used for the background.

### Example 4

### In Vitro Immunomodulatory Activities of Lanolin-related Compounds

Two separate in vitro assays were used to identify fractions of lanolin and related compounds that exhibit immune modulation activity. Fractions which gave higher than 20% suppression are considered positive responders in these assays.

Materials and Methods

A. T-Lymphocyte proliferation assay

T-lymphocytes were isolated from mouse thymus by standard methods, and suspended in DMEM (see, e.g., Mishell B B and Shiigi S M, eds., “Cell Proliferation” in Selected Methods in Cellular Immunology, V, XXIX, W. H. Freeman Co., San Francisco, Calif. pp. 153-160, 1980 and Dayton et al. (1992) Mol. Pharmacol. 41: 671-676). A total of 5×10⁶ cells/ml were incubated overnight at 37° C. in the presence of 3 μg/ml Con A to assess stimulation (positive score, related to 10 U/ml of rat interleukin-2) or suppression (negative score, related to control) of cell proliferation. Test substances were evaluated under these conditions at a pre-defined concentration. After overnight incubation, 2 μCi [³H]thymidine was added to each well. Cells were harvested after an additional 48 hours incubation, and thymidine incorporation assessed by liquid scintillation counting. Compounds used as positive control in the suppression assay were azathioprine (IC₅₀>10 μM), cyclophosphamide (IC₅₀>10 μM) and cyclosporin A (IC₅₀=0.012 μM. A parallel cytotoxicity assay was also conducted to verify that the suppression effect observed was not due to cell killing.

B. B-lymphocyte proliferation assay

B-lymphocytes were isolated from mouse spleen by standard methods, and suspended in DMEM. A total of 10⁶ cells/ml were incubated overnight at 37° C. in the presence of 1 μg/ml lipopolysaccharide (LPS) to assess stimulation (positive score, related to control) or suppression (negative score, related to control) of cell proliferation. Test substances were evaluated under these conditions at a pre-defined concentration. After overnight incubation, 2 μCi [³H]thymidine was added to each well. Cells were harvested. after an additional 48 hours incubation, and thymidine incorporation assessed by liquid scintillation counting. Compounds known to show positive effect on suppression are azathioprine (IC₅₀>10 μM), cyclophosphamide (IC₅₀>10 μM), and cyclosporin (IC₅₀=0.32 μM). A parallel cytotoxicity assay was also conducted to verify that the suppression effect observed was not due to cell killing.

Results

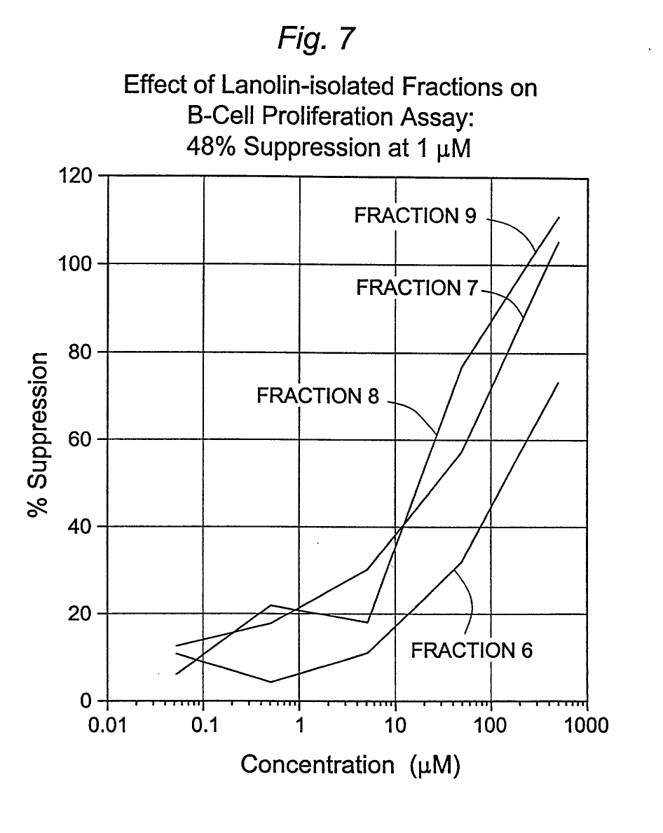

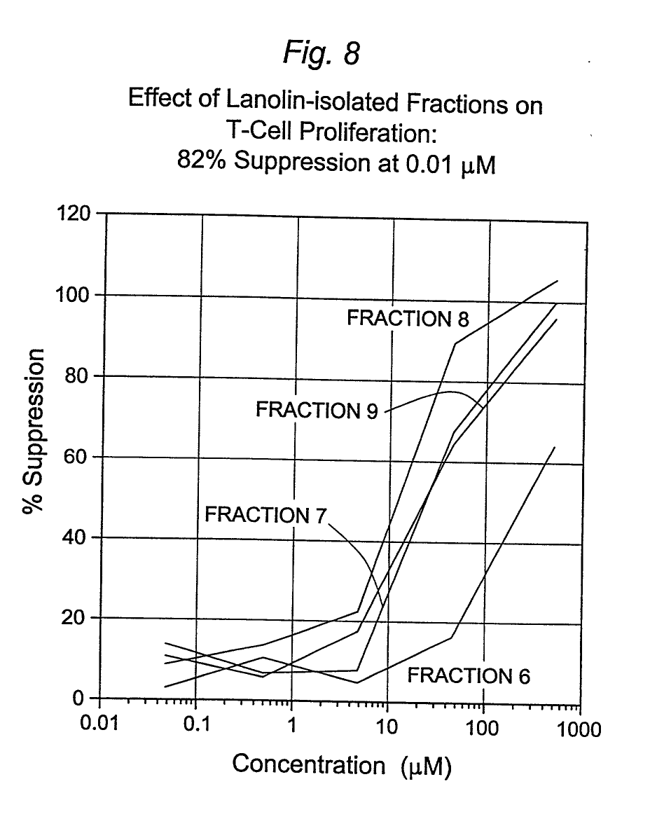

Initial studies were conducted using lanolin related materials and fractions isolated from lanolin. Results from the T-cell and B-cell assays (Table 10) indicate that 1) selected lanolin related materials, including lanolin USP, are active in blocking both T-cell and B-cell proliferation in vitro, 2) fractions 1 through 5 have no effect on either T-cell or B-cell proliferation, and 3) starting from fractions 6 through 9, there is a continuous increase in effectiveness of these fractions in suppressing both T-cell and B-cell proliferation, with fractions 8 and 9 having the highest activities. Dose response curves are shown for suppression of B cell proliferation (FIG. 7) and T cell proliferation FIG. 8). None of the test materials appear to affect cell viability indicated by the absence of cytotoxic effect in the assay. Therefore, these results indicate the polar fractions isolated from lanolin are effective in suppressing T-cell and B-cell proliferation.

TABLE 10
Effect of Lanolin-Related Compounds, and Fractions thereof,
on Lymphocyte Proliferation

| | T-cell Proliferation Assay | | B-cell Proliferation Assay | |
|---|---|---|---|---|
| | at 500 μM | | at 500 μM | |
| Material | Suppression | Cytotoxicity | Suppression | Cytotoxcity |
| | 81% | −6% | 78% | 11% |
| (+) Lanolin USP | 55% | 0% | 37% | 2% |
| | 78% | 5% | 80% | 3% |
| (+) Lanolin Oil | 65% | 1% | 70% | 17% |
| (+) Hydroxylated | 60% | 7% | 30% | 0% |
| Lanolin | | | | |
| | 3% | 4% | 8% | −6% |
| (+) Super Sterol Esters | 41% | 0% | 40% | 9% |
| LA-10400-13-01 | 26% | 10% | 28% | 3% |
| LA-10400-13-02 | −11% | 15% | 11% | 7% |
| LA-10400-13-03 | 2% | −3% | 6% | −7% |
| LA-10400-13-04 | 10% | −8% | 14% | −8% |
| LA-10400-13-05 | 2% | −4% | 11% | −11% |
| LA-10400-13-06 | 46% | −7% | 41% | −2% |
| LA-10400-13-07 | 83% | 24% | 108% | −26% |
| LA-10400-13-08 | 100% | 11% | 122% | −32% |
| LA-10400-13-09 | 101% | 19% | 126% | −35% |

### Example 5

### Animal Models for Inflammatory Conditions

Irritant Contact Dermatitis (ICD)

The phorbol ester 12-O-tetradecanoyl-13-phorbol acetate (TPA), the active ingredient in croton oil, induces skin inflammation which peaks between 24 and 30 hours when the ear is the target and between 8 and 16 hours when the flank is the target. These two alternative targets allow for observations of both short term and longer term activity of compounds in suppressing TPA-induced inflammation. The TPA swelling response is considered an acute irritant reaction mediated by eicosanoids and pro-inflammatory cytokine release from epidermal keratinocytes and infiltrating inflammatory cells and without a cell-mediated immune response.

Seven to eight week old female ICR mice (SKH-1, Charles River or Hr/Hr, Simonsen Labs) or ICR Swiss albino mice (Simonsen) are anesthetized and treated with 5 μl of a TPA working solution on each ear surface. The TPA working solution is made from a 10 mg/ml stock solution in dimethylsulfoxide and diluted 1:40 in ethanol to a final concentration of 0.25 μg/ul. One hour after TPA treatment, the ears are treated with the anti-inflammatory agent. Ear thickness measurements are taken before TPA treatment and before anti-inflammatory agent application, as well as at 6 hours, 24 hours and at 30 hours post-treatment, when the experiment is terminated. Ear swelling due to the acute irritant, TPA, reaches a peak between 24 and 30 hours. Alternatively, this treatment may be performed on the mouse flank using 25 μl of the same TPA solution; in this case, swelling of the flank reaches a peak between 8 and 16 hours, usually 8 hours. It should be noted that a more severe reaction to TPA in terms of erythema and eventual necrosis of the outer pinna is observed in the hairless mouse population compared with that of the ICR mouse population.

To test the efficacy of a potential anti-inflammatory agent, the agent can be incorporated into the solution containing TPA, with or without with excipients such as safflower oil, or avocado oil, and applied to the mouse ear with the irritant TPA. The effect of the anti-inflammatory can be measured by the ear swelling response as described above.

To test an anti-inflammatory agent in transdermal patch applications, the agent can be incorporated into the adhesive before casting the adhesive onto either the release liner, semi-permeable membrane or the backing material of a conventional transdermal patches. Alternatively, an anti-inflammatory agent can be incorporated into the drug reservoir and be applied to the skin concurrently. The irritation or allergic reactions due to the drug, the adhesive used, penetration enhancers, or solvents can be mitigated by the anti-inflammatory agent incorporated into the patch. In this approach, TPA can be used to model an irritant drug and the effect of the anti-inflammatory agent can be measured as inhibition of the swelling response induced by the patch.

Allergic Contact Dermatitis (ACD)

Allergic contact hypersensitivity (CH) is a clinically important type of dermatitis that can occur as a result of exposure to occupational or environmental agents. A number of chemicals can be used to experimentally reproduce this phenomenon. The sequence of events following initial application of a contact allergen to the skin (sensitization phase) is thought to involve presentation of the allergen in association with MHC class II molecules by the Langerhans cells (LCs). LCs migrate to the regional lymph nodes where they stimulate antigen-specific T cell proliferation. When the allergen is reapplied to the skin several days later (challenge phase), an allergic reaction occurs that takes 24-48 hours to develop. This type of response is termed delayed type hypersensitivity, in contrast to the immediate hypersensitivity mediated by mast cell degranulation.

Challenging mouse ears with the haptens 1-chloro-2,4-dinitrobenzene (DNCB), or 2,4-dinitrofluorobenzene (DNFB), after a 5 day sensitization phase, induces severe swelling associated with an immune response that includes a memory T cell infiltrate. Maximum swelling occurs between 24 and 48 hours after challenge. Thus, DNFB and DCNB are considered to induce delayed contact hypersensitivity.

The protocol involves shaving the posterior portion of the backs of anesthetized 7-8 week old female Balb/c mice with electric clippers. DNCB in acetone:olive oil (4:1), or DNFB in acetone alone is applied to the shaved area in a volume of 50 μl (DNCB) or 100 μl (DNFB). The concentration of DNCB in the sensitizing solution ranges from 0.5% to 4% w/v or 0.1 to 0.5% for DNFB. Five days after sensitization, the thickness of both ears is measured with a spring-loaded micrometer and both surfaces of the ears are challenged with either 10 μl of DNCB in acetone:olive oil ranging in concentration from 0.25%-2.5% w/v or 10 μl of DNFB in acetone at 0.1%, or vehicle alone. Post challenge ear thickness measurements are taken at 8, 16, 24, 32, 48, 56 and 72 hours. Typical ear thickness measurements are more variable than in the TPA model and depend on the sensitization and challenge doses of hapten as well on the individual groups of mice. However, a swelling response of at least 50% above baseline is considered reasonable. Although it is assumed that only the challenged ears will demonstrate swelling, the control ears will be used to identify any effects of sensitization and the vehicle on inflammation.

### Example 6

### Reduction of TPA- and DNFB-induced Inflammation by Administration of Lanolin

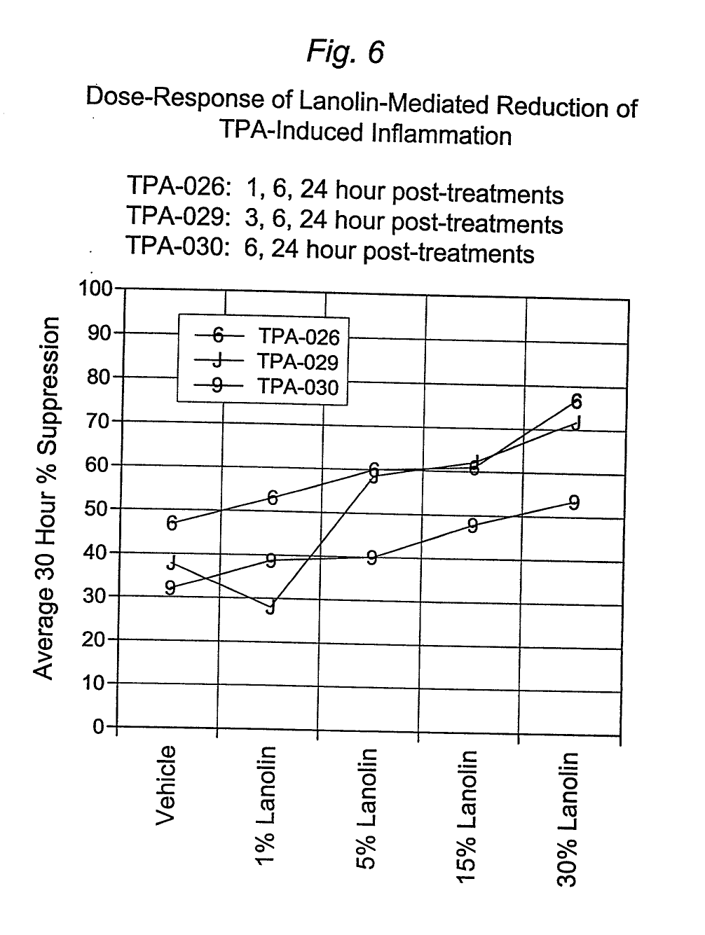

These experiments demonstrate the ability of lanolin to reduce both acute inflammation and delayed contact hypersensitivity inflammation. The model systems described in Example 5 were employed, using TPA challenge as a model for acute inflammation and DNFB challenge as a model for delayed contact hypersensitivity. One hour after challenge with TPA or DNFB, formulations containing up to 30% lanolin were applied to mice. The percent suppression for each formulation was determined as described in Example 5 at 30 hours post-challenge (for TPA) or 48 hours post-challenge (for DNFB). The results are reported as the percent suppression of inflammation, compared to untreated control animals, as measured by ear thickness. As shown in Table 11, lanolin reduced both TPA- and DNFB-induced inflammation in a dose-dependent manner up to 76% and 49%, respectively. Lanolin and lanolin oil (see below) typically suppress TPA-induced ear swelling to a greater extent than DNFB-induced inflammation. A dose-response curve of lanolin-mediated suppression of TPA-induced inflammation is also shown in FIG. 6.

### Example 7

### Reduction of TPA- and DCNB-induced Inflammation by Administration of Lanolin Oil

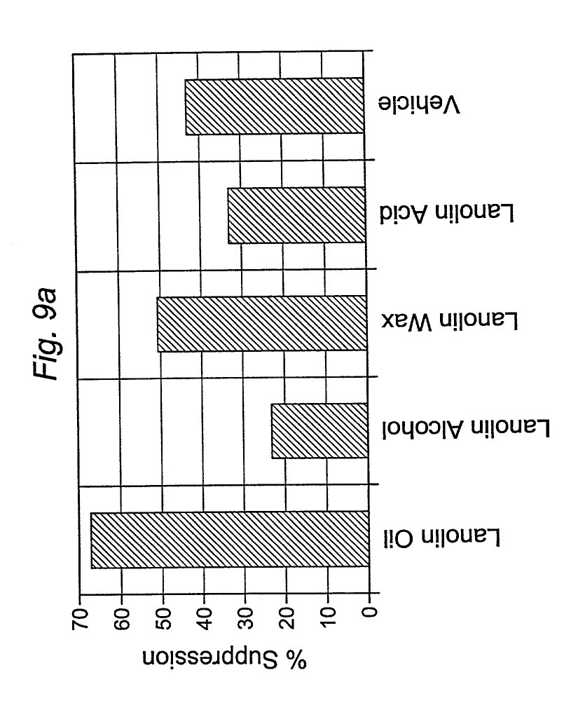

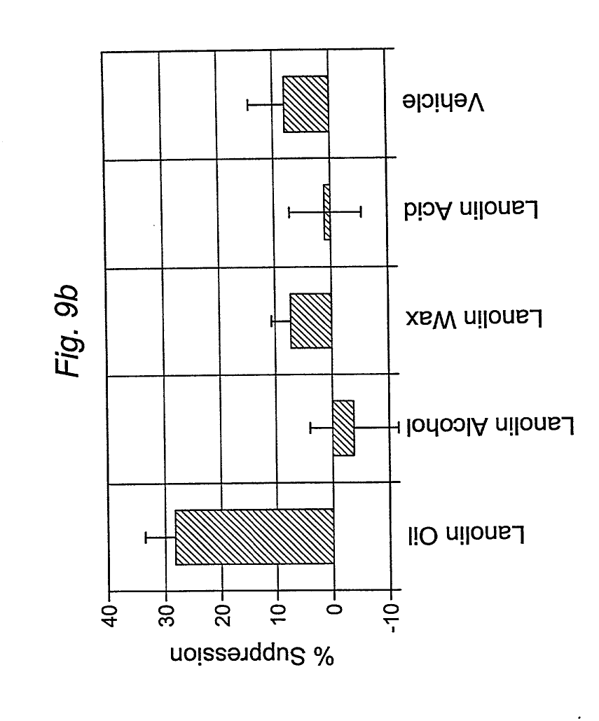

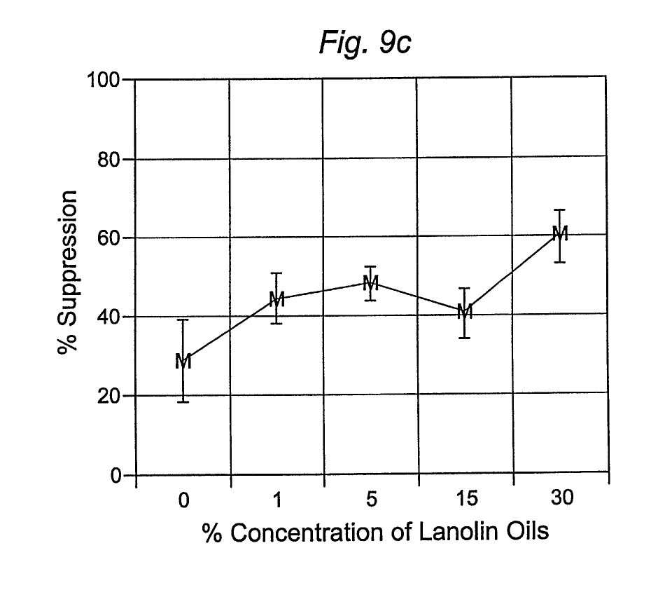

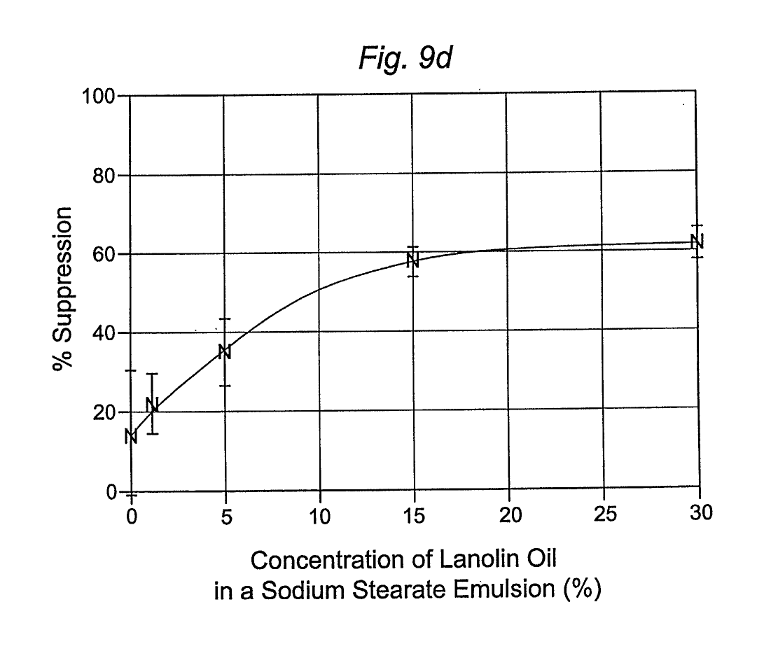

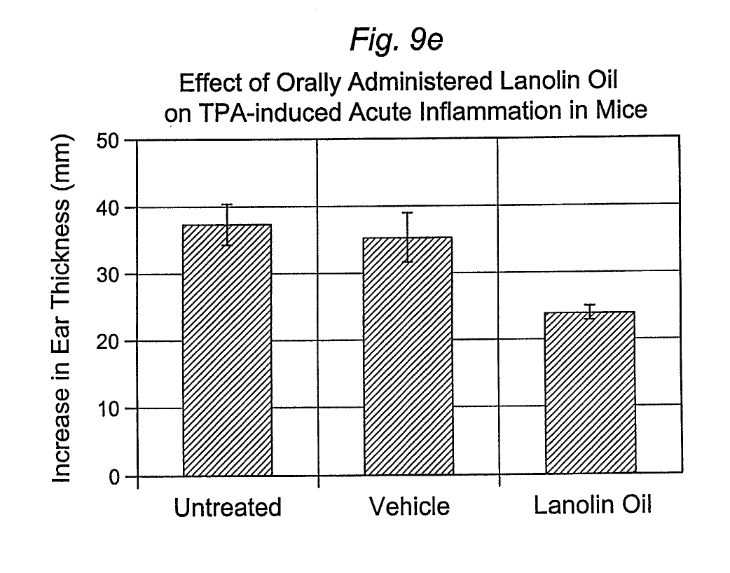

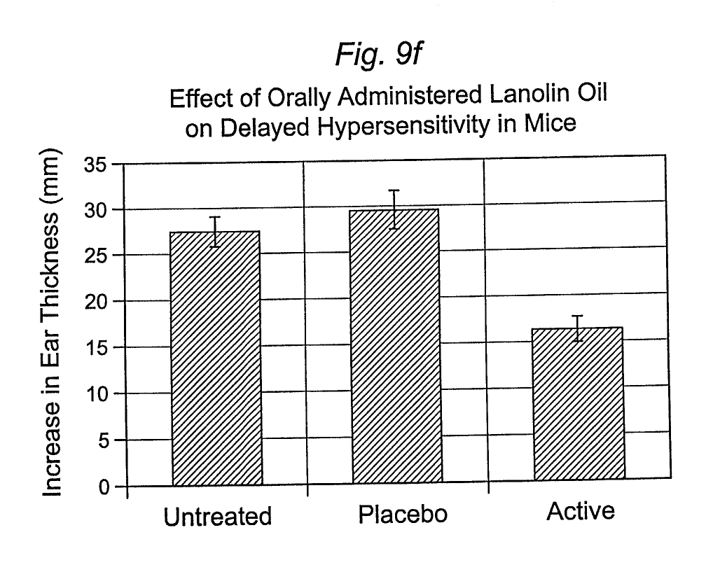

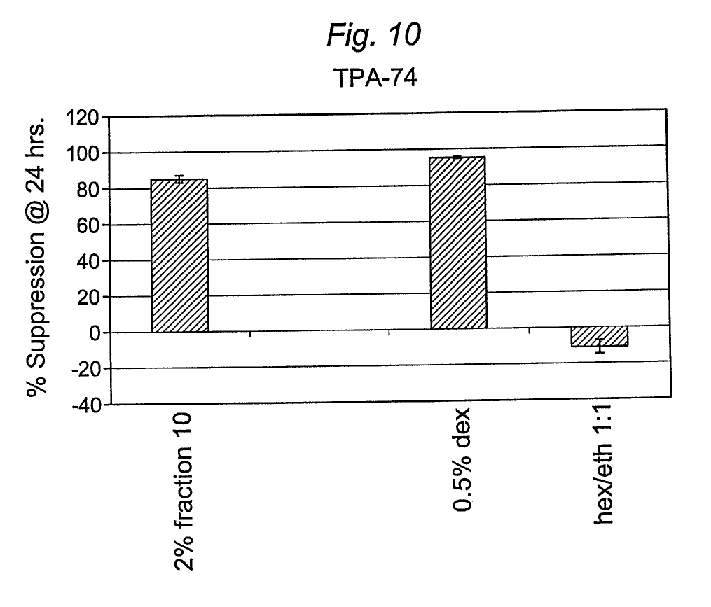

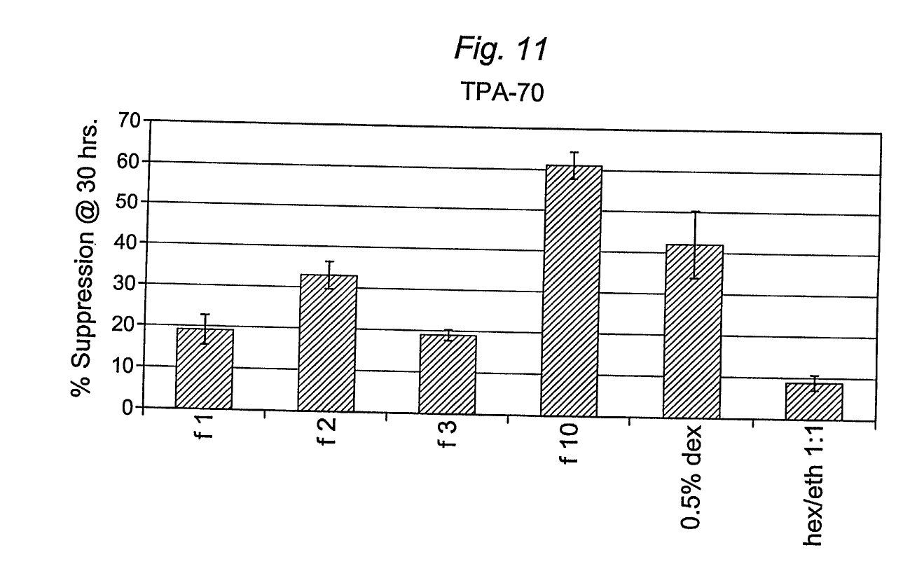

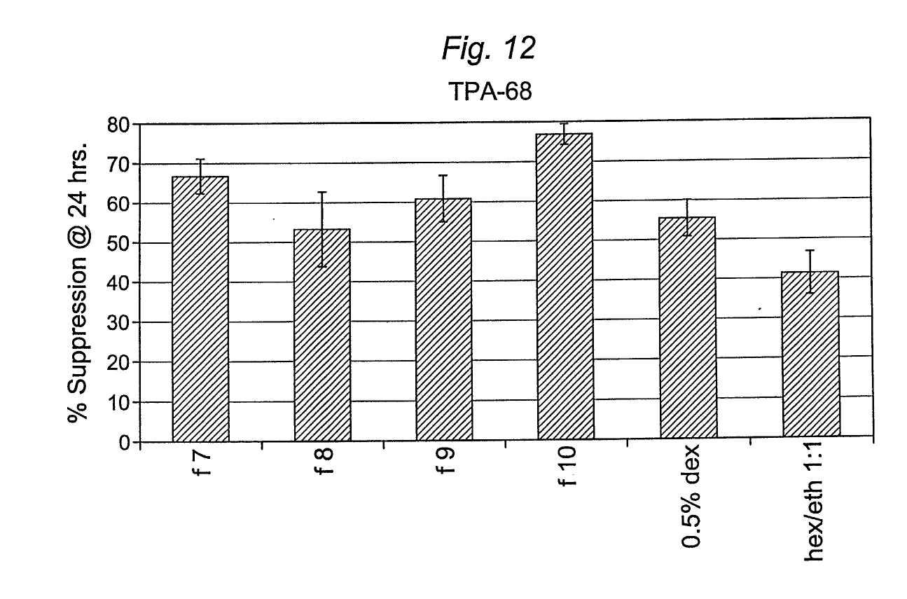

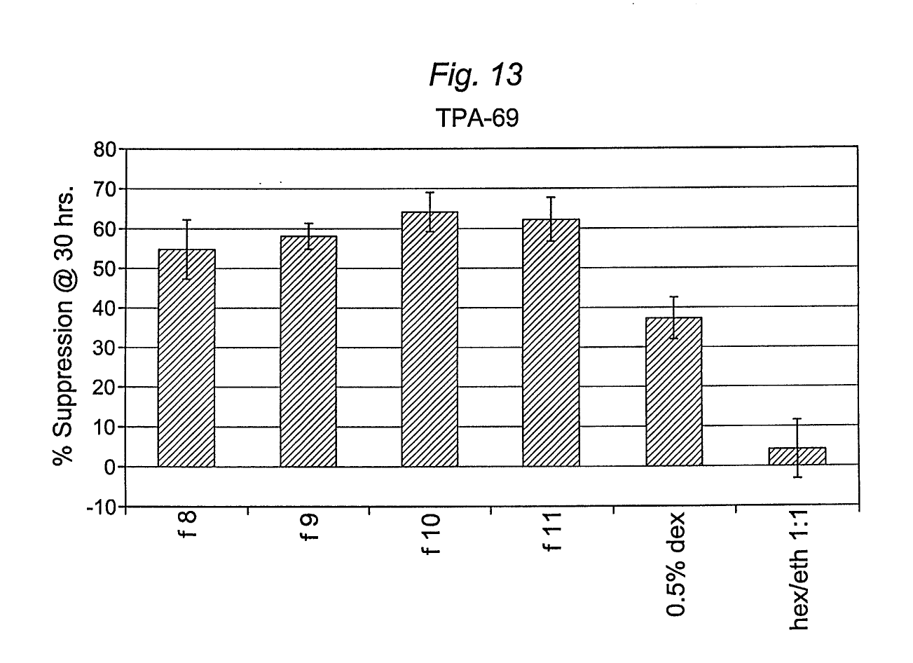

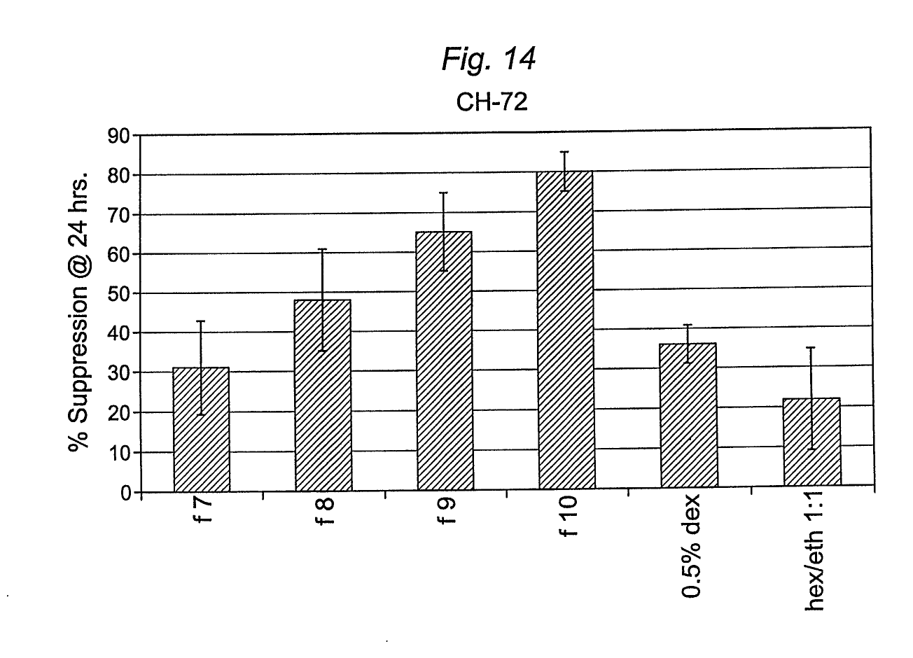

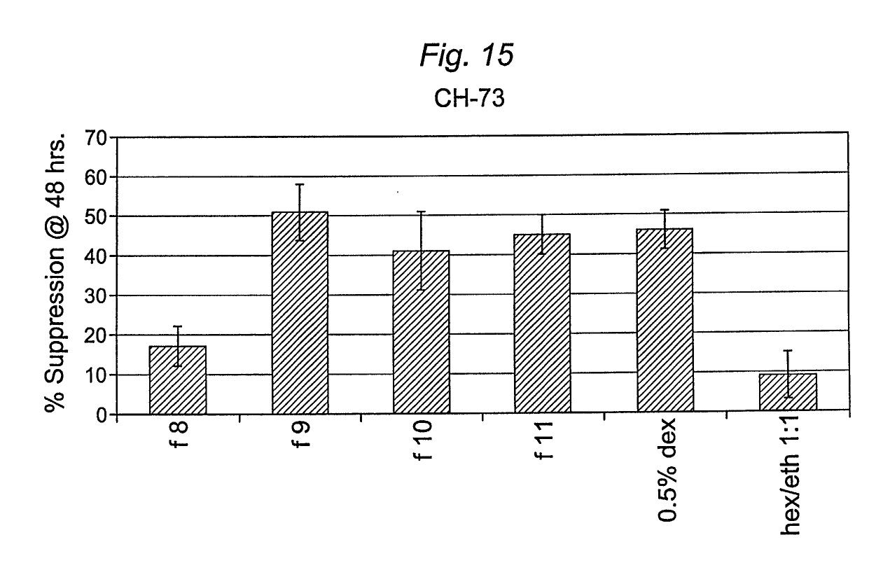

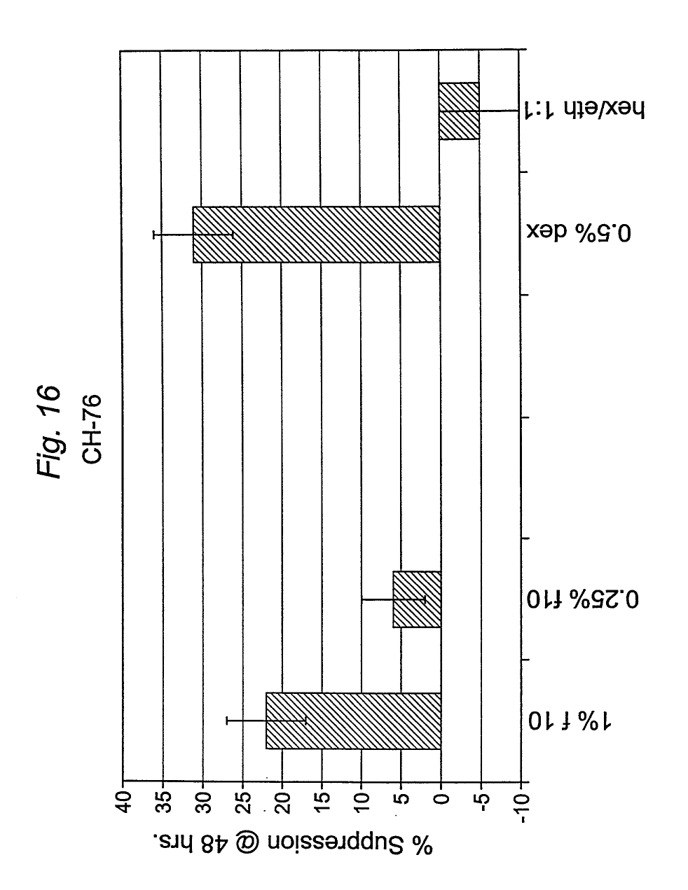

These experiments demonstrate that lanolin oil is also able to reduce both acute inflammation and delayed contact hypersensitivity inflammation. The model systems described in Example 5 were employed, using TPA challenge as a model for acute inflammation and DNFB challenge as a model for delayed contact hypersensitivity. One hour after challenge with TPA or DNFB, formulations containing up to 30% lanolin were applied to mice. The percent suppression for each formulation was determined as described in Example 5 at 30 hours post-challenge (for TPA) or 48 hours post-challenge (for DNFB). Results are reported as the percent suppression of inflammation, compared to untreated control animals, as measured by ear thickness. As shown in Table 11, lanolin oil suppressed both TPA- and DNFB-induced inflammation in a dose-dependent manner up to 62% and 50%, respectively. Lanolin oil was comparable to lanolin in suppressing the acute irritant reaction induced by TPA, whereas it was generally more potent in suppressing the DNFB-induced contact hypersensitivity. Lanolin oil (15%) incorporated into a stearic acid emulsion also reduced TPA-induced acute inflammation nearly 60% (FIG. 9D).

TABLE 11
Suppression of Inflammation in vivo by Lanolin and Lanolin Oil

| | TPA-Induced Acute | DNFB-Induced Delayed |
|---|---|---|
| | Irritant Reaction | Contact Hypersensitivity |
| | (Suppression at 30 hr) | (Suppression at 48 hr) |
| Lanolin (Conc.) | | |
| Vehicle Control | 26% | 12% |
| 1% | 53% | 2% |
| 5% | 55% | 6% |
| 15% | 60% | 27% |
| 30% | 76% | 49% |
| Lanolin Oil (Conc.) | | |
| Vehicle Control | 26% | 12% |
| 1% | 34% | 23% |
| 5% | 60% | 26% |
| 15% | 46% | 47% |
| 30% | 62% | 50% |

### Example 8

### Reduction of TPA- and DCNB-induced Inflammation by Administration of SuperSterol Esters

The ability of super sterol ester to suppress inflammation was determined. The model systems described in Example 5 were employed, using TPA challenge as a model for acute inflammation and DNFB challenge as a model for delayed contact hypersensitivity.

As shown in Table 12, super sterol ester exhibits anti-inflammatory activity in both animal models.

TABLE 12

| | | % Suppression | |
|---|---|---|---|
| | Concentration | (TPA) | % Suppression (CH) |
| Compound | (%) | (30 hr) | (48 hr) |
| Super Sterol Ester | 15% | 51% | 37% |
| | 10% | | 38% |
| | 5% | | 23% |

### Example 9

### Reduction of Glycolic Acid-Induced Skin Inflammation

Topical glycolic acid is known to have irritating effects in humans, which include erythema and edema. This experiment was designed to assess topical irritation in the SKH-1 hairless mouse using 8% glycolic acid lotions applied to the flanks with or without occlusion. The lotions also contained a stearic acid emulsion, a preservative, carbopol polymer, and purified water. An 8% concentration was selected since it has been well-recognized that ≧8% concentration of α-hydroxy acids (AHA), including glycolic acid, induce a high incidence of irritation in humans. Irritation was measured by determining transepidermal water loss (TEWL) with a Tewameter (Courage+Khazaka Electronics GmbH, Cologne, Germany) and visual inspection.

Four female SKH-1 mice, aged nine weeks, had AHA lotions applied to each flank with or without occlusion under a Finn chamber (Allerderm Laboratories, Petaluma, Calif.). After 24 hours, each flank was examined for signs of erythema or edema. Baseline and 24-hour TEWL readings were taken. Two mice had AHA lotions applied to each entire flank, without occlusion. Two mice had AHA lotions applied to two spots per flank under occlusion.

| Group | |
|---|---|
| 1 | CAB-96-330 |
| 2 | CAB-96-332 |

Solutions:

Anesthetic:

Anased/Ketaset/saline (0.8 mL/0.05 mL/0.15 mL)

Lotions:

a) CAB-96-330 is a formulation containing 8% glycolic acid and a 21% skin protectant blend that contained petrolatum, lanolin, safflower oil, cholesterol, glycerin.

b) CAB-96-332 is a formulation containing 8% glycolic acid in the same vehicle as used for CAB-96-330, but without skin protectant.

Both formulations were adjusted to approximately pH 2.9.

Results:

As shown in Table 13, CAB-96-332 (without skin protectant blend) increased TEWL significantly at 48 and 72 hours whereas, CAB-96-330 (containing 21% skin protectant blend) prevented any increase in TEWL due to the 8% glycolic acid. Furthermore, no erythema nor edema was observed in skin sites treated with CAB-96-330. Therefore, the skin protectant can minimize irritation induced by AHA-like glycolic acid.

TABLE 13
Petrolatum- and Lanolin-Mediated Reduction of
Glycolic Acid-induced Inflammation

| Mouse | | Pretreatment | 4-hour | 48-hour | 72-hour | 96-hour |
|---|---|---|---|---|---|---|
| # | Treatment | TEWL | TEWL | TEWL | TEWL | TEWL |
| 1 | CAB-96- | 6.8 | 19.2 | 23.9 | 13.6 | 21.3 |
| | 330 | | | | | |
| 1 | CAB-96- | 6.5 | 19.1 | 27.9 | 16.0 | 23.1 |
| | 330 | | | | | |
| 2 | CAB-96- | 6.7 | 18.2 | 54.8 | 67.2 | 47.0 |
| | 332 | | | | | |
| 2 | CAB-96- | 7.4 | 16.5 | 48.1 | 67.0 | 69.3 |
| | 332 | | | | | |

### Example 10

### Prevention or Reduction of Transdermal Drug Delivery System-Induced Irritation

This Example describes the use of the anti-inflammatory compositions of the invention to reduce or prevent inflammation caused by transdermal drug delivery systems. It has been well documented that transdermal therapeutic systems can cause local irritation in approximately 15-20% of patients, and occasionally, contact hypersensitivity reactions. For example, moderate irritation and allergic contact dermatitis are known to develop during 21-day consecutive use of clonidine transdermal patches in man for 21 consecutive days. (Catapres-TTS®) (Maibach HI, Contact Dermatitis 12:192-195 (1985)).

To prevent or to reduce these local adverse reactions due to the use of transdermal patches, an anti-inflammatory agent formulation as described herein is administered along with the drug being administered transdermally. Such drugs include, but are not limited to, diuretics (e.g., furosemide, spironolactone), antidiarrheal agents (e.g., loperamide, diphenoxylate) or calcium channel blockers (e.g., isradipine, nicardipine, verapamil, etc.). The drug is formulated in a topical gel dosage form and is administered in conjunction with a transdermal patch such as a clonidine transdermal patch (Catapres-TTS®).

Various combinations of pre-, co- and/or post-treatment regimen are tested to achieve the best result with respect to minimizing these adverse skin reactions. Testing can be conducted by pretreating the skin at the site of patch application with the anti-inflammatory formulation, for example) at a 50-200 μl per cm² dose three times during the day prior to the application of the patch. Alternatively, the anti-inflammatory formulation can be applied about two hours prior to the application of the patch, or can be applied concurrently with the patch, as well as following the removal of the patch. Local adverse skin reactions, i.e., relative irritancy potential (21-day cumulative irritancy assay) and allergic contact dermatitis potential (Draize repeat insult patch test assay) of the clonidine patches with and without co-administration of an anti-inflammatory formulation is compared.

Ten percent lanolin oil was incorporated into a standard acrylic polymer adhesive system in a simple adhesive transdermal patch device. The anti-inflammatory properties of the lanolin oil patch were tested in the mouse TPA-induced inflammation model where either the control patch or the patch containing lanolin oil was placed on the skin one hour following challenge with TPA. The incorporation of lanolin oil into the patch resulted in minimal loss of adhesive capability, compared to the acrylic adhesive itself. Furthermore, the incorporation of lanolin oil in the same acrylic adhesive patch reduced the inflammation induced by topical TPA treatment. Therefore, this Example demonstrates the effectiveness of lanolin oil incorporation on mitigating inflammation in a conventional transdermal patch system.

### Example 11

### Prevention of Surfactant-Induced Skin Inflammation-Induced by Lanolin

It has been shown previously that continuous exposure to surfactant used in many soap preparations led to the development of irritant contact dermatitis in man. In fact, a human soap chamber model has been developed to assess the irritancy potential of new soap products. (see, e.g., Frosch and Kligman (1979) J. Am. Acad. Dermtol. 1:35-41; and Babulak et al. (1986) J. Soc. Cosmet. Chem., 37:475-479). In order to test the effectiveness of these naturally derived materials in blocking surfactant-induced irritation, lanolin was incorporated into a conventional soap base and was tested against the soap base alone. Following a standard soap chamber test procedure, an 8% aqueous solution of either the conventional soap base or the soap base containing approximately 3.5% of lanolin was applied to human forearm under an occlusive chamber. Approximately 24 hours after the initial application, the chamber was removed and the underlying test site thoroughly rinsed with running tap water after which the forearm was patted dry. Two and half hours after removal of the occlusive chamber, clinical assessments was performed in an environmental chamber. In this case, the skin erythema level was measured based on the Draize score and was also recorded with a Minolta Chromameter (see reference Babulak S W, Rhein L D, Scala D D, Simmion A F, and Grove G L, “Quantitation of Erythema in a Soap Chamber Test Using Minolta Chroma (Reflectance) Meter: Comparison of Instrumental Results with Visual Assessment, J. Soc. Cosmet. Chem., 37:475-479, 1986). Results from these studies are shown below. It is apparent that lanolin was effective in blocking surfactant-induced erythema in man.

### Example 12

### Isolation and Testing of Sub-fractions of Fraction 8

Fraction 8 isolated from lanolin or anti-inflammatory materials from other natural sources can be further purified by an HPLC method. An HPLC method was used to separate fraction 8 into another 40 fractions (Table 14); all fractions were collected in a microtiter plate.

TABLE 14

| Column: | Spherex 5 diol (5 μm, 250-460 mm) |
|---|---|
| Amount injected: | 1.5 mg |
| Injection Volume: | 20 μL (EtOAc Solution) |
| Flow Rate: | 1 ml/min |
| Solvents: | A = EtOAc |
| | B = Hexane |
| Time (minutes): | Gradient Condition |
| 0 | 10% A, 90% B |
| 0-30 | 40% A, 60% B |
| 30-40 | 100% A |
| Collection Time: | 1 min |
| Total Fractions: | 40 |

All 40 fractions were subsequently tested in the ConA-induced T-cell proliferation assay, the same assay described in Example 4. Fractions providing greater than or equal to 40% suppression are considered active. Results from this assay showed that fractions 21, 23, 26, 32, 33, 34, 35 and 39 were all effective in suppressing con-A induced T-cell proliferation.

### Example 13

### Inhibition of IL-2 Secretion from Human PBMC

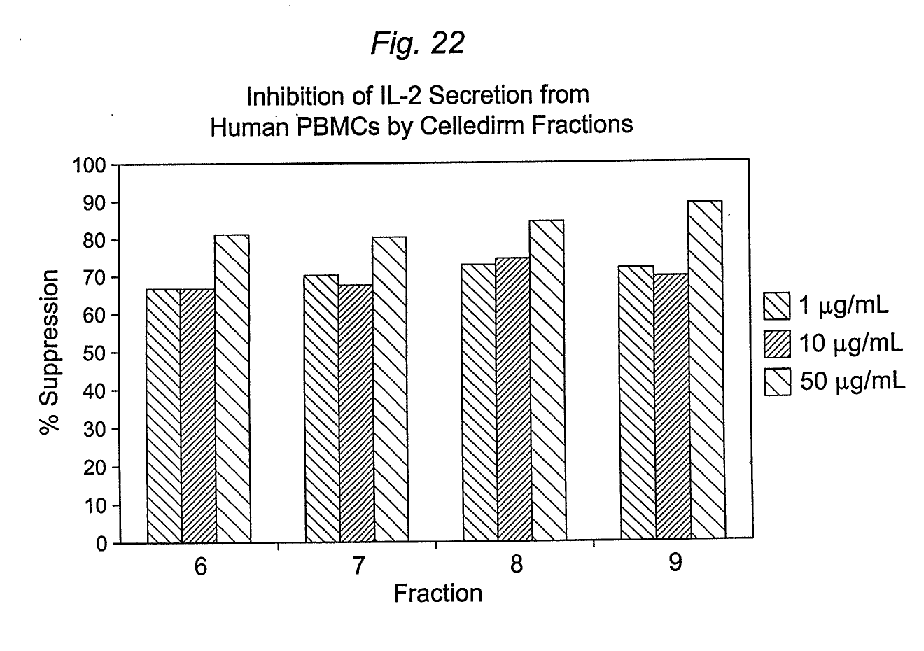

In this example, the ability of lanolin fractions to inhibit PHA-stimulated IL-2 production from human peripheral blood mononuclear cells (PBMCs) was assayed. IL-2 production from human PBMC was stimulated by phytohemagglutanin (20 μg/mL) based on a procedure described by Konno et al. (Konno S. I. et al. “Inhibition of Cytokine Production from Human Peripheral Blood Leukocytes by Anti-allergic Agents in vitro.” Eur. J. Pharmacol, 264:265-268, 1994) wherein IL-2 levels were measured 48 hours post-stimulation with a commercially available enzyme-immuno assay (EIA) kit and spectrophotometric measurements were performed with a microplate reader (MRX, Dynatech.) The various compositions of the present invention were tested at final concentrations of 1, 10, and 50 μg/m, in duplicate. Dexamethasone was used as the positive control in these studies; the IC₅₀ of dexamethasone was 5 nM. The levels of suppression caused by the test compounds are expressed as the percent decrease relative to the vehicle control values. The data for Fractions 6-9 are shown in FIG. 22.

### Example 14

### Inhibition of TNF-α Production from Human PBMC

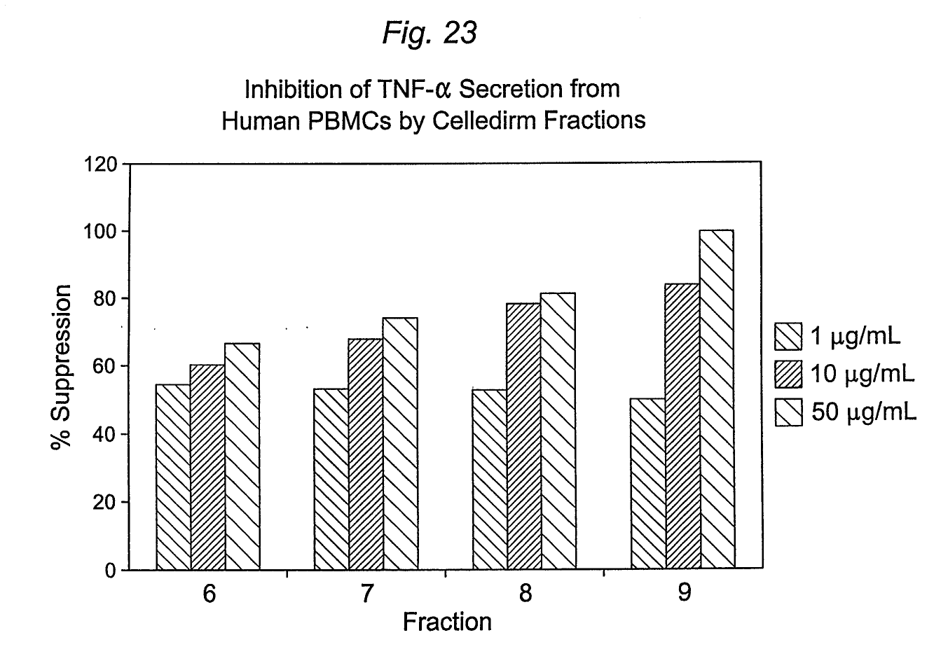

TNF-α production by human PBMCs was stimulated using lipopolysaccharide (1 μg/mL) based on a procedure by Schindler et al. (Schindler R et al. Correlations and Interactions in the Production of Interleukin (IL)-6, IL-1, and Tumor Necrosis Factor (TNF) in Human Blood Mononuclear Cells: IL-6 Suppresses IL-1 and TNF. Blood, 75:40-47, 1990). The levels of TNF-α were measured 24 hours following LPS stimulation with a commercially available enzyme-immuno assay (EIA) kit and spectrophotometric measurements were performed with a microplate reader (MRX, Dynatech). The compositions of the present invention were tested in this study were 1, 10, and 50 μg/mL in duplicate. Dexamethasone was used as the positive control; IC₅₀ of dexamethasone was typically 16 nM. The suppression caused by test compounds is expressed as the percent decrease relative to the vehicle control value. The results for fractions 6-9 are set forth in FIG. 23.

### Example 15

### In Vitro T-cell Anti-Proliferative and Cytotoxic Assay

In this example, the anti-proliferative and cytotoxic properties of Fraction 1A-12A were assayed on ConA-stimulated murine thymic T-cells and cultured A431 cells, respectively.

Materials and Methods

A. T-Lymphocyte proliferation assay

T-lymphocytes were isolated from mouse thymus by standard methods, and suspended in DMEM (see, e.g., Mishell B B and Shiigi S M, eds., “Cell Proliferation” in Selected Methods in Cellular Immunology, V, XXIX W. H. Freeman Co, San Francisco, Calif. pp. 153-160, 1980 and Dayton et al. (1992) Mol. Pharmacol. 41: 671-676). A total of 1×10⁶ cells/mL were pre-incubated in Con A at 2 μg/mL for one hour prior to the addition of the test compounds. Test substances were evaluated under these conditions at pre-defined concentrations. Cells were harvested 72 hours after incubation with test compounds, and evaluated using two methods, i.e., 1) visual microscopic evaluation and colorimetric evaluation to establish viable cell number at 570 nm using a MTT assay. The compound used as a positive antiproliferative control was rapamycin. As shown in Table 15, the anti-proliferative activity of lanolin resided predominantly in fractions 10A to 12A.

B. Cytotoxic Assay using human Epidermoid carcinoma cells

A parallel cytotoxicity assay was conducted to verify that the anti-proliferative effect of test substances on the T-cells was not due to cell killing. Using a cytotoxic assay on A431 human epidermoid carcinoma cells, the cytotoxicity of Fraction 1A-12A on A431 cells was evaluated. As shown in Table 15, the lanolin fractions were largely non-cytotoxic. A431 cell cytotoxicity was restricted primarily to fraction 12A.

TABLE 15
Effects of Lanolin Fractions on T Cell Proliferation and Cell Viability

| | |
|---|---|
| T Cell Proliferation | |
| (% Inhibition) | Cytotoxicity (% of Control) |

| Fraction | 50 μg/ml | 10 μg/ml | 50 μg/ml | 10 μg/ml |
|---|---|---|---|---|
| 1A | −12 | −4 | 100 | 99 |
| 2A | −21 | 6 | 99 | 95 |
| 3A | −5 | −4 | 95 | 101 |
| 4A | −11 | −9 | 92 | 94 |
| 5A | 10 | −17 | 96 | 100 |
| 6A | −12 | −15 | 94 | 98 |
| 7A | −20 | −5 | 99 | 102 |
| 8A | −1 | 6 | 103 | 95 |
| 9A | 28 | 10 | 89 | 93 |
| 10A | 82 | 62 | 85 | 97 |
| 11A | 86 | 79 | 75 | 102 |
| 12A | 85 | 19 | 74 | 86 |

### Example 16

### Suppression of Single Dose Ultraviolet B-Induced Erythema

Human subjects of skin type I to III are first screened for previous history of skin disorders where patients with atopic eczema are excluded from the study. Subjects qualified for the study are first photo-patch tested to define the individual minimal erythema dose (MED) of UVB. Four test areas are marked on the buttock skin before the study; the first site is not treated but UV irradiated, one is treated with a placebo vehicle without a concentrated inflammation modifiers and UV irradiated and the third is treated with a concentrated inflammation modifier in the placebo vehicle and UV irradiated. Finally, a no treatment and not UV irradiated site is used as a baseline comparison. Treatments can be applied 30 minutes before the UV irradiation and two and ten hours after the UV irradiation. Twenty four hours after UV irradiation, the levels of erythema are measured based on Draize score. Subsequently, 4 mm skin biopsies are removed from all four sites of the skin whereas the degree of inflammation is determined histopathologically. In addition to quantifying the number of infiltrating inflammatory cells, the levels of TNF-α, IL-1 and ICAM-1 (Intercellular Adhesion Molecule-1) expression will be assessed immunohistochemically, similar to a procedure reported earlier (see, Nickoloff B. J., et al., J. Am. Acad. Dermatol., 30(4):535-46 (1994), “Perturbation of Epidermal Barrier Function Correlates with Initiation of Cytokine Cascade in Human Skin. J. Am. Acad. Dermatol., 30:535-546, 1994). Furthermore, the levels of matrix metalloproteinase will be measured as an indication of the ability of concentrated inflammation modifiers on dermal matrix alteration, and thus, signs of skin aging. The overall erythema and inflammation readings among all four treatment sites will be evaluated to substantiate the anti-inflammatory properties of the concentrated inflammation modifiers.

### Example 17

### Suppression of Ultraviolet B-induced Skin Inflammation in Mice

A dose of 0.47 J/cm² given to hairless mice (127 seconds at 4 inches from the bulbs to the table top) provides a good swelling response (approximately two-fold over baseline at 24 hours) that can be overcome by the application of sunblock with an SPF of 15. This dose does not cause erythema or wound-like lesions on the skin and, therefore, can be considered an ideal dose for inflammation studies. In this experiment, effective concentrated inflammation modifier formulations, as determined by their ability to reduce swelling induced by TPA, will be tested for their ability to reduce swelling induced by UVB radiation.

Five hairless female mice were anesthetized and the right flanks covered with a piece of opaque tape with three 7/16″ cutouts. All mice were irradiated at the same time for 127 seconds at 4″ from the bulbs to the table top to produce an effective dose of 0.047 J/cm² or 1 MED (minimal edema dose). The irradiated spots were demarcated with a marker pen and the tape removed. The spots were painted with 1 formulation per mouse immediately after irradiation and at 8 hours, 24, 32 and 48 hours after irradiation. The swelling response will be measured also at these times. One mouse will be treated with UVB alone. Formulation CAB-96-295 contains concentrated inflammation modifiers; commercial products “Eterna 27” and Halog, a corticosteroid were used as comparison.

CAB-96-295, a formulation containing concentrated inflammation modifiers, was effective in blocking inflammation induced by UVB. CAB-96-295 was more effective than Halog, a commercially available glucocorticoid steroid in blocking UVB-induced inflammation in mice.

TABLE 16
Suppressing UVB-induced Inflammation in Mice

| Treatment | % Suppression at 24-hour |
|---|---|
| Halog | 28.5 + 18.9 |
| Eterna 27 | 3.8 + 8.46 |
| CAB-96-304 | −10.64 + 11.70 |
| CAB-96-295 | 49.4 + 7.6 |

### Example 18

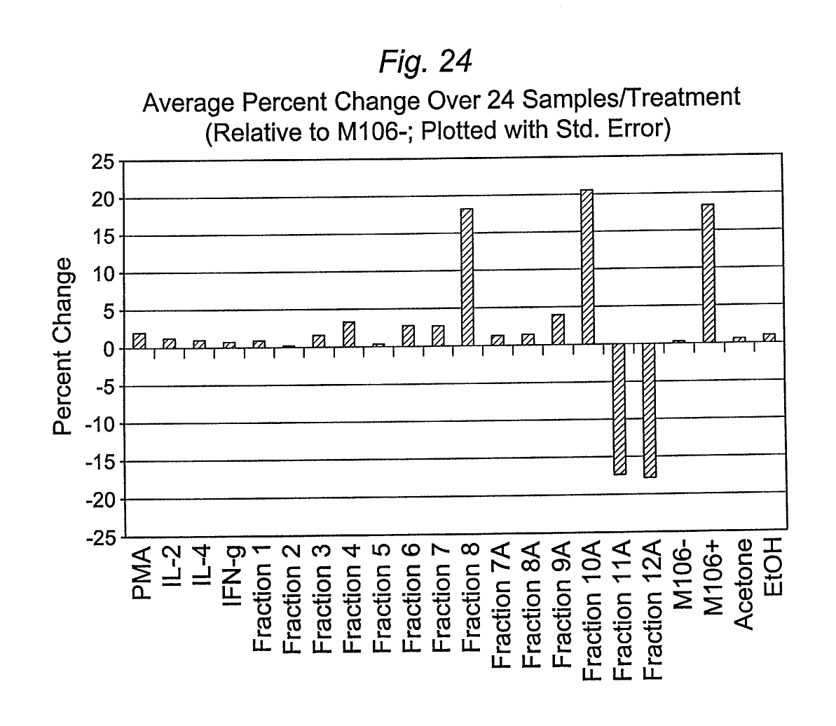

In this Example, various lanolin-derived fractions were tested for their ability to enhance the growth of primary human dermal fibroblasts in culture. Results shown in FIG. 24 suggest that fractions 8 and 10 A have profound effects on promoting cell viability. In most individual experiments, cell viability was increased 66-188%, compared to cells incubated in the absence of M106 growth factor supplement. When OD values for each treatment were averaged over 6 separate plates, (4 wells/plate×6 plates=24 wells), fractions 8 and 10A appear to be as good as M106 in promoting cell viability.

### Example 19

### Suppression of Multiple Ultraviolet B Exposures-Induced Erythema

In this Example, human subjects of skin type I to III are first screened for previous history of skin disorders where patients with atopic eczema are excluded from the study. Subjects qualified for the study are first photo-patch tested to define the individual minimal erythema dose (MED) of UVB. Four test areas are marked on the buttock skin before the study; the first site is not treated but UV irradiated, one is treated with placebo vehicle without a concentrated inflammation modifier and UV irradiated and the third one is treated with a concentrated inflammation modifier in the placebo vehicle and UV irradiated. Finally, a no treatment and no UV-irradiated site is used as a baseline comparison. Treatments can be applied twice daily for two consecutive weeks whereas three of the four sites are UV irradiated three times weekly, i.e. Monday, Wednesday and Friday after the morning treatment phase for two consecutive weeks. At the end of the two-week study period, the levels of erythema and pigmentation are measured based on Daize score and by Minolta Chronometer. Subsequently, 4 mm skin biopsies are removed from all four sites of the skin whereas the degree of inflammation is determined histopathologically. In addition to quantitate the actual number of infiltrating inflammatory cells, the levels of TNF-α, IL-1 and ICAM-1 (Intercellular Adhesion Molecule-1) expression will be assessed immunohistochemically, similar to a procedure reported earlier. (see, Nickoloff B. J., et al., J. Am. Acad. Dermatol., 30(4):535-46 (1994); “Perturbation of Epidermal Barrier Function Correlates with Initiation of Cytokine Cascade in Human Skin. J. Am. Acad. Dermatol., 30:535-546, 1994). The overall erythema and inflammation reading among all four treatment sites will be compared to substantiate the anti-inflammatory property of concentrated inflammation modifiers.

### Example 20

### Effect of Concentrated Inflammation Modifiers for Photodamaged Skin

Patients with clinically moderate to severe photoaging of the face and forearm are recruited to a 48-week study design to assess the efficacy of the concentrated inflammation modifiers of the present invention. The concentrated inflammation modifiers are applied once or twice daily to the entire face and forearms for 48 weeks. Clinical evaluation are performed at baseline; after two weeks of treatment; and then monthly until the end of the study. The overall clinical response at each visit, compared with the baseline, was rated as worse, 1; no change, 2; slightly improved, 3; improved, 4; much improved, 5. Overall severity of photoaging and individual features of photoaging, including fine and coarse wrinkling, mottled hyperpigmentation, and actinic letigines, were assessed at baseline and at each clinical visit. An ordinal scale 0 through 9 was used for these assessments, where 0 indicates no evidence of the parameter in question; 1 through 3, mild severity; 4 through 6, moderate severity; and 7 through 9, severe.

In order to assess the skin improvement histologically, a 2-mm punch biopsy specimen was taken from one periorbital area of each patient, at baseline and at the end of treatment. After standard tissue processing, various parameters, including stratum corneum compaction, spongiosis, granular layer thickness, dermal matrix protein organization, and inflammatory cell infiltration were assessed. Statistically significant improvement from baseline, either based on clinical scores or on histological assessment is an indication that concentrated inflammation modifier is effective in preventing and/or reversing the signs and symptoms of skin aging.

### Example 21

### Solid-Phase Extraction with Silica Gel

A solution of 5.14 g of lanolin oil in 15 mL of hexane was slowly poured in portions into a round-bottom flask containing 5 g of silica gel. During the addition of the lanolin oil solution, the flask was periodically swirled to effect thorough mixing/dispersal of the solution within the silica gel. The flask was then placed on a rotary evaporator and the residual hexane was removed. The remaining solid was transferred to an Erlenmeyer flask and 200 mL of hexane was added to it. The resulting slurry was stirred at approximately 50° C. for 1 hr. The silica gel was filtered off, and the hexane solution evaporated to provide approximately 2.32 g (app 45%) of thick oil containing the less polar phase of lanolin oil. The filtered silica gel was placed into an Erlenmeyer flask and to it was added 100 mL of ethyl acetate. This slurry was stirred at 50° C. for 1 hr, followed by filtration. Rotary evaporation of the ethyl acetate solution afforded 1.17 g (app. 23%) of a very thick, yellow oil comprising the more polar phase of lanolin oil. (Thin layer chromatography analysis of the extracts was performed using 60:40 hexane/ethyl acetate eluent, silica gel plate, developed with 2% aq. sulfuric acid. R_{f} Values: Less-polar extract—major spot at R_{f} 0.64; minor spot at 0.35; More-polar extract—major spots at R_{f} 0.35, 0.13, 0.08, and 0.06; minor spot at R_{f} 0.64.

### Example 22

### Subfrationation of Fraction 10A Using a Chromatotron

Fraction 10A can be further separated into 12 sub-fractions designated Fractions Chr1-Chr12 using a mixed solvent system, such as hexane:ethylacetate (60:40). In this technique, centrifugal thin layer chromatography (Chromatotron) is used to further fractionate fraction 10A. The subfractions were then tested in the in vitro T-cell anti-proliferative and cytotoxic assays as set forth in Example 15. Subfraction Chr1 was inactive in the T-cell proliferation assay (not shown). Significant activity was detected in Chr3, which intensified in Chr4-Chr12 (Table 17). None of the chromatotron data exhibited significant cytotoxicity.

TABLE 17

| | T-cell Proliferation Assay | | | |
|---|---|---|---|---|
| | Suppression | | Cytotoxicity | |
| | OD 570 nm | | OD 570 nm | |
| SUBFRACTION | 50 μg/mL | media | 50 μg/mL | media |
| Chr. 2 | 0.44 | 0.554 | 0.672 | 0.668 |
| Chr. 3 | 0.136 | 0.554 | 0.62 | 0.668 |
| Chr. 4 | 0.076 | 0.554 | 0.633 | 0.668 |
| Chr. 5 | 0.069 | 0.572 | 0.658 | 0.704 |
| Chr. 6 | 0.075 | 0.572 | 0.704 | 0.704 |
| Chr. 7 | 0.077 | 0.572 | 0.698 | 0.704 |
| Chr. 8 | 0.071 | 0.572 | 0.67 | 0.704 |
| Chr. 9 | 0.062 | 0.53 | 0.651 | 0.628 |
| Chr. 10 | 0.069 | 0.53 | 0.683 | 0.628 |
| Chr. 11 | 0.062 | 0.53 | 0.64 | 0.628 |
| Chr. 12 | 0.062 | 0.53 | 0.59 | 0.628 |

### Example 23

### Fractionation of Lanolin Oil using Supercritical Fluid Extraction

This example shows the extraction of lanolin oil using a supercritical fluid extraction method. The extraction vessel is charged with 100.7 grams of lanolin oil. Carbon dioxide gas is liquefied at 2200 psi and 80° C. using a compressor and a heat exchanger. The liquid carbon dioxide is fed into one end of the extraction vessel using a slow feed rate (˜1 g/min). The liquid carbon dioxide is collected with the lanolin oil extract at the other end of the extraction vessel by passing it through the pressure reduction valve. A 0.7-gram (0.5-1.5 gm) fraction containing the odor was collected and discarded. Next, the pressure of the liquefied carbon dioxide was increased to 4200 psi at 80° C. A new collection flask was placed and the remaining deodorized lanolin oil was extracted with liquid carbon dioxide at 4200 psi and a temperature of 80° C. A total of 31.0 grams (25-40 gm) of concentrated test material was collected. There were 62.0 grams of depleted material which remained in the extraction vessel that was biologically inactive and discarded. The concentrated test material typically produced anti-inflammatory and anti-allergic activities in the in vitro T- cell proliferation with an IC₅₀ of 7.5 μg/ml (5-15 μg/ml).

### Example 24

### Comparison of Biological Efficacy with Various Fractionation Procedures

This Example compares various fractions from different chromatographic procedures. Fraction 10A is a lanolin derived fraction obtained using gravity column chromatography. It is largely cholesterol and more polar constituents. It is a dark amber, tacky semi-solid. Fractions 9A-11A are lanolin derived fractions obtained using gravity column chromatography similar to Fraction 10A, but also contain material that are less polar than cholesterol. It is an amber color and less tacky than Fraction 10A. SFE-2 is a lanolin oil super critical fluid extraction fraction. It contains cholesterol and less polar constituents. It is a thick, light amber liquid with little odor.

TABLE 18
Biological Efficacy

| | |
|---|---|
| In Vitro | In Vivo |

| Product | T-cell | Cytotoxic | TPA | DNFB |
|---|---|---|---|---|
| Fraction 10A | +++ | Negative: | +++ | +++ |
| | | A431, Jukat | | |
| Fraction 9A-11A | ++ | Negative: A431 | ++ | ++ |
| SFE-2 | ++ | Negative: A431 | ++ | ++ |

All fractions, especially Fraction 10A, are active product both in vitro and in vivo. Fraction 10A inhibits T cell proliferation (ConA-stimulated mouse thymocyte assay) half maximally at about 2.5 μg/ml. None of the fractions are cytotoxic to A431 cells (epidermoid carcinoma line). In addition, Fraction 10A is not cytotoxic to Jurkat cells (T cell lymphoma line), primary thymic epithelial cells, or primary skin fibroblasts. Fraction 10A stimulates fibroblast growth, even in the absence of growth factors. All the products are active in the TPA-induced acute inflammation model.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A composition comprising a therapeutic agent which causes inflammation and a concentrated inflammation modifier, wherein the concentrated inflammation modifier comprises at least one polar chromatographic fraction of a fat, oil, or wax from a plant, animal, or mineral source and wherein administration of said therapeutic agent in conjunction with said concentrated inflammation modifier results in an inflammatory response, as measured in an ear swelling assay, that is reduced by at least about 50% compared to said inflammatory response induced by administration of said therapeutic agent in the absence of said concentrated inflammation modifier.

2. The composition according to claim 1, wherein said therapeutic agent is administered as a topical dosage form.

3. The composition according to claim 1, wherein said composition inhibits proliferation of T cells or B cells.

4. The composition according to claim 1, wherein said concentrated inflammation modifier comprises at least one chromatographic fraction of a fat, oil, or wax obtained from a sebaceous secretion of a ruminant.

5. The composition according to claim 4, wherein said ruminant is selected from the group consisting of cattle, sheep, goats, llamas, alpaca, and deer.

6. The composition according to claim 5, wherein said concentrated inflammation modifier comprises at least one chromatographic fraction of a wool fat-related compound.

7. The composition according to claim 6, wherein said wool fat-related compound is selected from the group consisting of lanolin, lanolin oil, and super sterol ester.

8. The composition according to claim 1, wherein said fat, oil, or wax is obtained from a plant source.

9. The composition according to claim 8, wherein said fat, oil, or wax is a member selected from the group consisting of: aloe vera leaf oil, olive oil, castor oil, apricot kernel oil, avocado oil, grain germ oils, candelilla wax, carnauba wax, carrot oil, soybean oil, shea butter, nut oils, palm kernel oil, jojoba oil, rape seed oil, vegetable oil, sesame oil, sunflower oil, safflower oil, corn oil, canola oil, rice bran oil, rose hip seed oil, coconut oil, amyl butyrate, camphor, 1-carvone, cotton seed oil, eugenol, menthol, oil of anise, oil of clove, orange oil, peanut oil, peppermint oil, rose oil, shark liver oil, soybean oil, walnut oil, and cocoa butter.

10. The composition according to claim 1, wherein said fat, oil, or wax is obtained from a mineral-based source.

11. The composition according to claim 10, wherein said mineral-based source is a member selected from the group consisting of paraffin products, petrolatum and mineral oil.

12. The composition according to claim 1, wherein said polar chromatographic fractions are obtained using a chromatographic method selected from the group consisting of a silica gel column, silica gel solid phase extraction, high pressure liquid chromatography, thin layer chromatography, and super critical fluid extraction.

13. The composition according to claim 1, wherein said polar chromatographic fractions are obtained using a silica gel column.

14. The composition according to claim 13, wherein said polar chromatographic fraction is obtained by loading said fat, oil, or wax onto said silica gel column in a first solvent and eluting said fractions having modulatory activity from said silica gel by contacting said silica gel with a second solvent having a greater polarity than said first solvent.

15. The composition according to claim 14, wherein said silica gel is washed using a solvent having a polarity intermediate to those of said first solvent and said second solvent.

16. The composition according to claim 14, wherein said fraction comprises at least one of fractions 6, 7, 8 and 9; and wherein
fraction 1, is eluted from said column using a second solvent having a polarity equivalent to that of dichloromethane:hexanes (about 2.5:97.5);
fraction 2, is eluted from said column using a second solvent having a polarity equivalent to that of dichloromethane:hexanes (about 5:95 followed by about 10:90) after elution using a solvent having a polarity equivalent to that of dichloromethane:hexanes (about 2.5:97.5);
fraction 3, is eluted from said column using a second solvent having a polarity equivalent to that of ethyl acetate:hexanes (about 2:98) after elution using a solvent having a polarity equivalent to that of dichloromethane:hexanes (about 10:90);
fraction 4, is eluted from said column using a second solvent having a polarity equivalent to that of ethyl acetate:hexanes (about 3:97) after elution using a solvent having a polarity equivalent to that of ethyl acetate:hexanes (about 2:98);
fraction 5, is eluted from said column using a second solvent having a polarity equivalent to that of ethyl acetate:hexanes (about 5:95) after elution using a solvent having a polarity equivalent to that of ethyl acetate:hexanes (about 3:97);
fraction 6, is eluted from said column using a second solvent having a polarity equivalent to that of ethyl acetate:hexanes (about 10:90) after elution using a solvent having a polarity equivalent to that of ethyl acetate:hexanes (about 5:95);
fraction 7, is eluted from said column using a second solvent having a polarity equivalent to that of ethyl acetate:hexanes (about 25:75) after elution using a solvent having a polarity equivalent to that of ethyl acetate:hexanes (10:90);
fraction 8, is eluted from said column using a second solvent having a polarity equivalent to that of ethyl acetate (about 100%) after elution using a solvent having a polarity equivalent to that of ethyl acetate:hexanes (about 25:75); and
fraction 9, is eluted from said column using a second solvent having a polarity equivalent to that of methanol:ethyl acetate (about 10:90) after elution using a solvent having a polarity equivalent to that of ethyl acetate (about 100%).

17. The composition of claim 16, wherein said column is equilibrated with 100% hexanes, and said fat, oil, or wax is dissolved in 100% hexanes prior to loading on said column.

18. The composition of claim 16, wherein said fat, oil, or wax is a wool fat-related compound selected from the group consisting of lanolin and lanolin oil.

19. The composition of claim 18, wherein said wool fat-related compound is lanolin and fraction 6 is a medium yellow wax, fraction 7 is a deep yellow wax/oil, fraction 8 is a yellow viscous oil, and fraction 9 is a yellow viscous oil/wax.

20. The composition in accordance with claim 14, wherein said fraction comprises at least one of fractions 8A, 9A, 10A, 11A and 12A; and wherein
fraction 8A, is eluted from said column using a second solvent having a polarity equivalent to that of ethyl acetate:hexanes (about 10:90) after elution using a first solvent having a polarity equivalent to that of ethyl acetate:hexanes (about 5:95) to remove fractions 1A to 7A;
fraction 9A, is eluted from said column using a second solvent having a polarity equivalent to that of ethyl acetate:hexanes (about 25:75) after elution using a solvent having a polarity equivalent to or less than that of ethyl acetate:hexanes (10:90);
fraction 10A, is eluted from said column using a second solvent having a polarity equivalent to that of ethyl acetate:hexanes (about 50:50) after elution using a solvent having a polarity equivalent to that of ethyl acetate:hexanes (25:75);
fraction 11A, is eluted from said column using a second solvent having a polarity equivalent to that of ethyl acetate (about 100%) after elution using a solvent having a polarity equivalent to that of ethyl acetate:hexanes (about 50:50); and
fraction 12A, is eluted from said column using a second solvent having a polarity equivalent to that of methanol (about 100%) after elution using a solvent having a polarity equivalent to that of ethyl acetate (about 100%).

21. The composition in accordance with claim 13, wherein said silica gel in said silica gel column is silica gel having about 63 mesh to about 200 mesh and is about 60 Å and is in a column.

22. A composition in accordance with claim 20, wherein said composition comprises fraction 10A.

23. A composition in accordance with claim 20, wherein said composition comprises a member selected from the group consisting of fractions 9A, 10A, 11A, or mixtures thereof.

24. A composition comprising a fraction obtained by chromatographic separation of a fat, oil, or wax, wherein said fraction is prepared by:
loading said fat, oil, or wax onto a silica gel column in a solvent having a polarity that is less than ethyl acetate:hexanes (5:95);
eluting undesired components of said fat, oil, or wax using a first eluant selected from the group consisting of ethyl acetate:hexanes (5:95), ethyl acetate:hexanes (10:90), ethyl acetate:hexanes (25:75), and ethyl acetate (100%); and
eluting said fraction from said column using at least one second eluant selected from the group consisting of ethyl acetate:hexanes (10:90), ethyl acetate:hexanes (25:75), ethyl acetate (100%), and methanol:ethyl acetate (10:90), wherein said second eluant is more polar than said first eluant.

25. The composition of claim 24, wherein said fat, oil, or wax is loaded onto said column in 100% hexanes.

26. The composition of claim 24, wherein said first eluant is ethyl acetate:hexanes (about 5:95) and said second eluant is methanol:ethyl acetate (about 10:90).

27. The composition of claim 24, wherein said first eluant is ethyl acetate:hexanes (about 10:90) and said second eluant is methanol:ethyl acetate (about 10:90).

28. The composition of claim 24, wherein said first eluant is ethyl acetate:hexanes (about 25:75) and said second eluant is methanol:ethyl acetate (about 10:90).

29. The composition of claim 24, wherein said first eluant is ethyl acetate:hexanes (about 25:75) and said second eluant is ethyl acetate (about 100%).

30. The composition of claim 24, wherein said first eluant is ethyl acetate (about 100%) and said second eluant is methanol:ethyl acetate (about 10:90).

31. The composition in accordance with claim 20, wherein Fraction 8A (a) is a yellow wax; and (b) has an infrared transmittance spectrum as shown in FIG. 17.

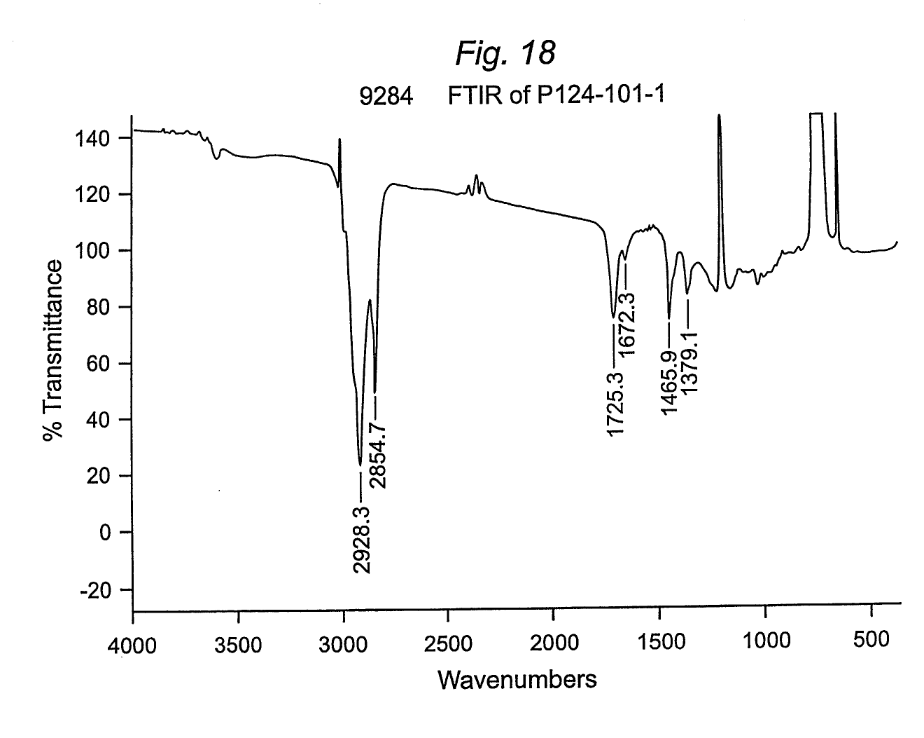

32. The composition in accordance with claim 20, wherein Fraction 9A (a) is a yellow wax; and (b) has an infrared transmittance spectrum as shown in FIG. 18.

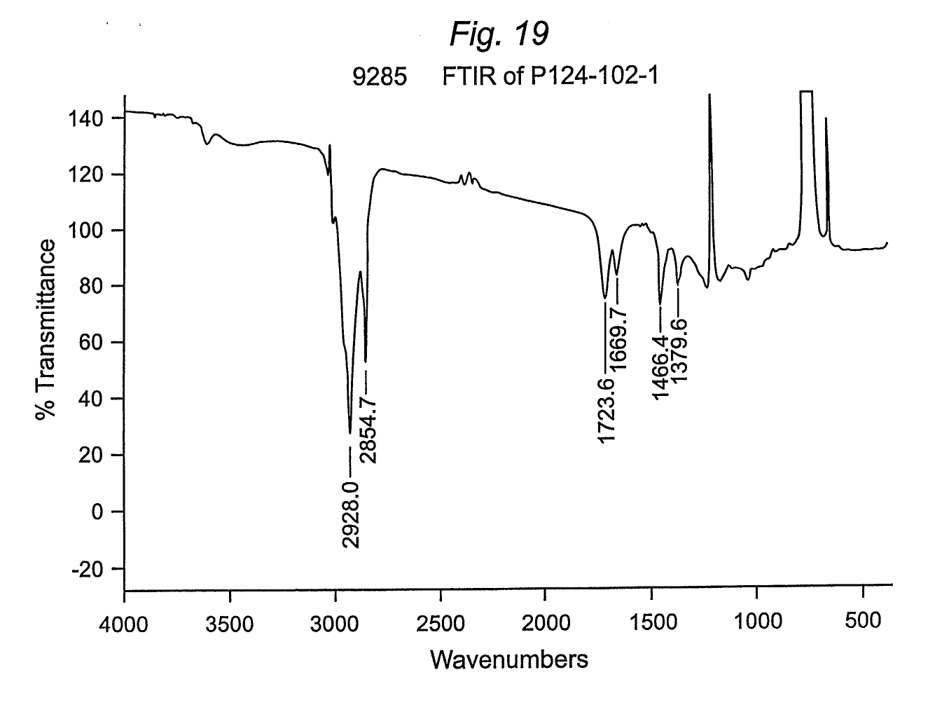

33. The composition in accordance with claim 20, wherein Fraction 10A (a) is a yellow wax or dark yellow wax; and (b) has an infrared transmittance spectrum as shown in FIG. 19.

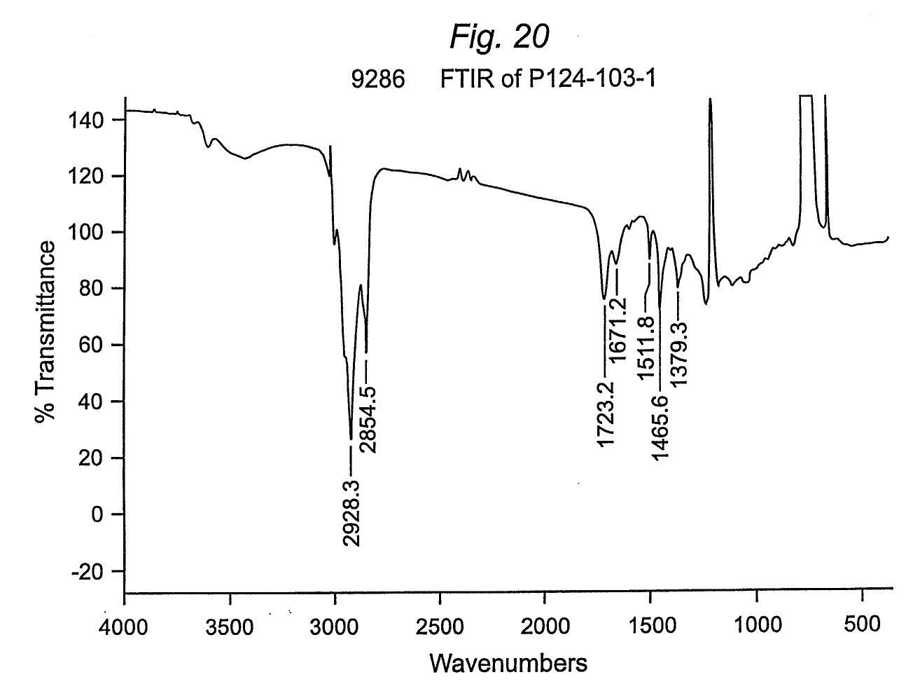

34. The composition in accordance with claim 20, wherein Fraction 11A (a) is a dark yellow wax; and (b) has an infrared transmittance spectrum as shown in FIG. 20.

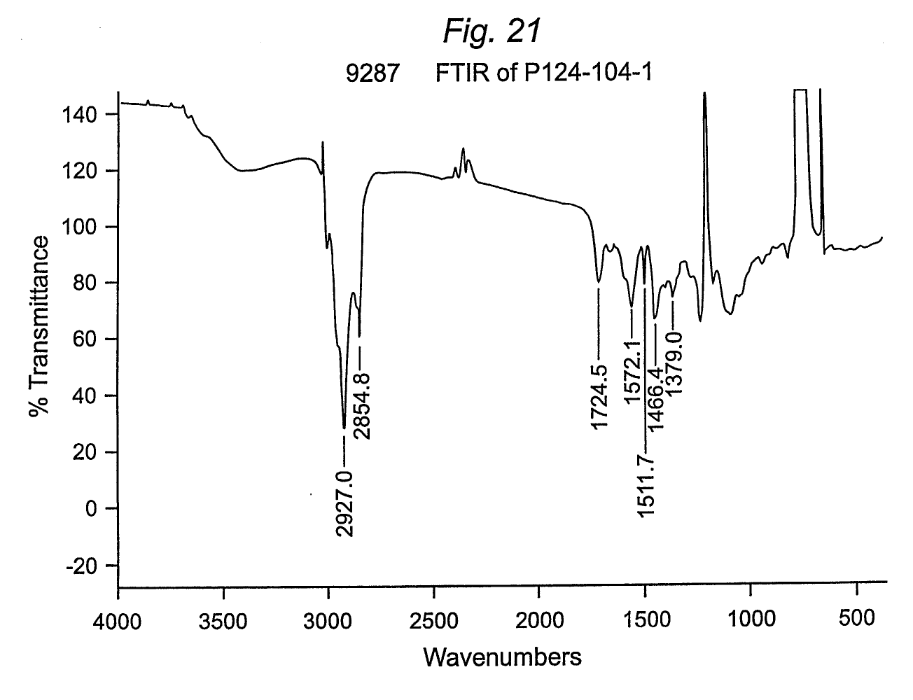

35. The composition in accordance with claim 20, wherein Fraction 12A (a) is a dark yellow wax; and (b) has an infrared transmittance spectrum as shown in FIG. 21.

36. A method of modulating an immune response in a mammal, said method comprising:
administering to said mammal a composition comprising a concentrated inflammation modifier which comprises at least one polar chromatographic fraction of a fat, oil, or wax from a plant, animal, or mineral source.

37. The method of claim 36, wherein said fat, oil, or wax is a wool fat-related compound selected from the group consisting of lanolin, lanolin oil, and super sterol ester.

38. The method of claim 36, wherein said chromatographic fraction is obtained by chromatography on a silica gel column.

39. The method of claim 38, wherein said chromatographic fraction is obtained by loading said fat, oil, or wax onto said silica gel column in a first solvent and eluting said fractions having modulating activity from said silica gel by contacting said silica gel with a second solvent having a greater polarity than said first solvent.

40. The method of claim 38, wherein said silica gel is washed using a solvent having a polarity intermediate to those of said first solvent and said second solvent.

41. The method of claim 38, wherein said fraction is prepared by:
loading said fat, oil, or wax onto a silica gel column in 100% hexanes;
eluting undesired components of said fat, oil, or wax using a first eluant selected from the group consisting of ethyl acetate:hexanes (about 5:95), ethyl acetate:hexanes (about 10:90), ethyl acetate:hexanes (about 25:75) and ethyl acetate (about 100%); and
eluting said fraction from said column using at least one second eluant selected from the group consisting of ethyl acetate:hexanes (about 10:90), ethyl acetate:hexanes (about 25:75), ethyl acetate (about 100%), and methanol:ethyl acetate (about 10:90), wherein said second eluant is more polar than said first eluant.

42. The method of claim 36, wherein said immune response is associated with a condition selected from the group consisting of skin sensitization, skin irritation, or inflammation.

43. The method of claim 42, wherein said condition is an inflammatory condition which is selected from the group consisting of irritant contact dermatitis, allergic contact dermatitis, T-cell mediated skin disorders, and skin disorders that are caused by local inflammatory mediator release.

44. A method of inhibiting proliferation of a B- or T-lymphocyte, said method comprising:
contacting a cell containing said lymphocyte with a composition comprising a concentrated inflammation modifier which comprises at least one chromatographic fraction of a fat, oil, or wax from a plant, animal, or mineral source.

45. The method of claim 44, wherein said fraction is prepared by:
loading said fat, oil, or wax onto a silica gel column in 100% hexanes;
eluting undesired components of said fat, oil, or wax using a first eluant selected from the group consisting of ethyl acetate:hexanes (about 5:95), ethyl acetate:hexanes (about 10:90), ethyl acetate:hexanes (about 25:75) and ethyl acetate (about 100%); and
eluting said fraction from said column using at least one second eluant selected from the group consisting of ethyl acetate:hexanes (about 10:90), ethyl acetate:hexanes (about 25:75), ethyl acetate (about 100%), and methanol:ethyl acetate (about 10:90), wherein said second eluant is more polar than said first eluant.

46. A method of administering a skin inflammation-inducing drug to skin, said method comprising:
administering to said skin a composition comprising said drug and a concentrated inflammation modifier in an amount effective to reduce said drug-induced skin inflammation, wherein said concentrated inflammation modifier comprises a polar fraction obtained by chromatographic separation of a fat, oil, or wax obtained from a plant, animal, or mineral source.

47. The method of claim 46, wherein said fraction is prepared by:
loading said fat, oil, or wax onto a silica gel column in 100% hexanes;
eluting undesired components of said fat, oil, or wax using a first eluant selected from the group consisting of ethyl acetate:hexanes (about 5:95), ethyl acetate:hexanes (about 10:90), ethyl acetate:hexanes (about 25:75) and ethyl acetate (about 100%); and
eluting said fraction from said column using at least one second eluant selected from the group consisting of ethyl acetate:hexanes (about 10:90), ethyl acetate:hexanes (about 25:75), ethyl acetate (about 100%), and methanol:ethyl acetate (about 10:90), wherein said second eluant is more polar than said first eluant.

48. The method of claim 46, wherein said concentrated inflammation modifier is administered prior to application of said drug.

49. A method of administering a skin inflammation-inducing drug to skin, said method comprising:
administering said drug to said skin in conjunction with an electric current, wherein drug-induced inflammation is reduced by co-administering with said drug an effective amount of a concentrated inflammation modifier, wherein said concentrated inflammation modifier comprises a polar fraction obtained by chromatographic separation of a fat, wax, or oil obtained from a plant, animal, or mineral source.

50. The method of claim 49, wherein said fraction is prepared by:
loading said fat, oil, or wax onto a silica gel column in 100% hexanes;
eluting undesired components of said fat, oil, or wax using a first eluant selected from the group consisting of ethyl acetate:hexanes (about 5:95), ethyl acetate:hexanes (about 10:90), ethyl acetate:hexanes (about 25:75) and ethyl acetate (about 100%); and
eluting said fraction from said column using at least one second eluant selected from the group consisting of ethyl acetate:hexanes (about 10:90), ethyl acetate:hexanes (about 25:75), ethyl acetate (about 100%), and methanol:ethyl acetate (about 10:90), wherein said second eluant is more polar than said first eluant.

51. The method of claim 49, wherein said concentrated inflammation modifier is administered prior to administration of said drug.

52. The method of claim 49, wherein said concentrated inflammation modifier is administered after administration of said drug.

53. A method of treating skin sensitization, said method comprising: administering to a sensitized area an effective amount of a composition comprising a concentrated inflammation modifier, wherein said concentrated inflammation modifier comprises a polar chromatographic fraction obtained from a fat, wax, or oil obtained from a plant, animal, or mineral source.

54. The method of claim 53, wherein said concentrated inflammation modifier comprises at least one chromatographic fraction of a fat, oil, or wax from a plant, animal, or mineral source, wherein said chromatographic fraction is prepared by:
loading said fat, oil, or wax onto a silica gel column in 100% hexanes;
eluting undesired components of said fat, oil, or wax using a first eluant selected from the group consisting of ethyl acetate:hexanes (about 5:95), ethyl acetate:hexanes (about 10:90), ethyl acetate:hexanes (about 25:75) and ethyl acetate (about 100%); and
eluting said fraction from said column using at least one second eluant selected from the group consisting of ethyl acetate:hexanes (about 10:90), ethyl acetate:hexanes (about 25:75), ethyl acetate (about 100%), and methanol:ethyl acetate (about 10:90), wherein said second eluant is more polar than said first eluant.

55. A method of preventing skin sensitization induced by administration of an agent that induces skin sensitization, said method comprising administering to a sensitized area an effective amount of a concentrated inflammation modifier, wherein said concentrated inflammation modifier comprises a polar chromatographic fraction obtained from a fat, wax, or oil obtained from a plant, animal, or mineral source.

56. The method of claim 55, wherein said fraction is prepared by:
loading said fat, oil, or wax onto a silica gel column in 100% hexanes;
eluting undesired components of said fat, oil, or wax using a first eluant selected from the group consisting of ethyl acetate:hexanes (about 5:95), ethyl acetate:hexanes (about 10:90), ethyl acetate:hexanes (about 25:75) and ethyl acetate (about 100%); and
eluting said fraction from said column using one or more second eluants selected from the group consisting of ethyl acetate:hexanes (about 10:90), ethyl acetate:hexanes (about 25:75), ethyl acetate (about 100%), and methanol:ethyl acetate (about 10:90), wherein said second eluant is more polar than said first eluant.

57. A method for modulating healing of a wound, said method comprising: administering to said wound a concentrated inflammation modifier, wherein said concentrated inflammation modifier comprises a polar fraction obtained by chromatographic separation of a fat, oil, or wax from a plant, animal, or mineral source.

58. The method of claim 57, wherein said fraction is prepared by:
loading said fat, oil, or wax onto a silica gel column in 100% hexanes;
eluting undesired components of said fat, oil, or wax using a first eluant selected from the group consisting of ethyl acetate:hexanes (about 5:95), ethyl acetate:hexanes (about 10:90), ethyl acetate:hexanes (about 25:75) and ethyl acetate (about 100%); and
eluting said fraction from said column using at least one second eluant selected from the group consisting of ethyl acetate:hexanes (about 10:90), ethyl acetate:hexanes (about 25:75), ethyl acetate (about 100%), and methanol:ethyl acetate (about 10:90), wherein said second eluant is more polar than said first eluant.

59. The method of claim 57, wherein said modulating is enhancing wound healing.

60. The method of claim 57, wherein said modulating is preventing the progression of wound development.

61. A method for minimizing an inflammatory reaction due to application of a topical cosmetic, cosmeceutical, dermatological, or other dosage form, said method comprising:
administering to said site of administration of said dosage form a concentrated inflammation modifier in an amount effective to reduce the amount of skin or mucosal membrane inflammation induced by said dosage form, wherein said concentrated inflammation modifier comprises a composition comprising a polar fraction obtained by chromatographic separation of a wool fat-related compound.

62. The method of claim 61, wherein said fraction is prepared by:
loading said fat, oil, or wax onto a silica gel column in 100% hexanes;
eluting undesired components of said fat, oil, or wax using a first eluant selected from the group consisting of ethyl acetate:hexanes (about 5:95), ethyl acetate:hexanes (about 10:90), ethyl acetate:hexanes (about 25:75) and ethyl acetate (about 100%); and
eluting said fraction from the column using at least one second eluant selected from the group consisting of ethyl acetate:hexanes (about 10:90), ethyl acetate:hexanes (about 25:75), ethyl acetate (about 100%), and methanol:ethyl acetate (about 10:90), wherein said second eluant is more polar than said first eluant.

63. The method of claim 61, wherein said topical dosage form comprises an active ingredient which is a member selected from the group consisting of retinoic acid, and a retinoic acid derivatives

64. The method of claim 61, wherein said topical dosage form comprises one or more α-hydroxy acids as an active ingredient.

65. The method of claim 64, wherein said α-hydroxy acid is glycolic acid.

66. The method of claim 64, wherein said α-hydroxy acid is lactic acid.

67. A method of inhibiting proliferation of a T-lymphocyte, said method comprising:
contacting a cell comprising said T-lymphocyte with a concentrated inflammation modifier, wherein said concentrated inflammation modifier comprises a polar fraction obtained by chromatographic separation of a fat, oil, or wax obtained from a plant, animal, or mineral source.

68. A method for inhibiting TNF-α secretion from a cell, said method comprising:
contacting said cell with a concentrated inflammation modifier, wherein said concentrated inflammation modifier comprises a polar fraction obtained by chromatographic separation of a fat, oil, or wax obtained from a plant, animal, or mineral source.

69. A method for retarding the aging of skin, said method comprising:
contacting said skin with a concentrated inflammation modifier, wherein said concentrated inflammation modifier comprises a polar fraction obtained by chromatographic separation of a fat, oil, or wax obtained from a plant, animal, or mineral source.

70. The method of claim 69, wherein said aging is caused by ultraviolet radiation.

71. The method of claim 69, wherein said concentration inflammation modifier further comprises a member selected from the group consisting of retinoic acid, retinoic acid derivatives, or a related compound, as an active ingredient.

72. A composition comprising a concentrated inflammation modifier, wherein the concentrated inflammation modifier comprises one or more polar chromatographic fractions of a fat, oil, or wax from a plant, animal, or mineral source; and an antiinflammatory agent.

73. The composition of claim 72, wherein said antiinflammatory agent is a member selected from the group consisting of such as glucocorticoid, Vitamin D3, methotrexate, cyclosporine alpha-hydroxy acids, retinoic acid and retinoic acid derivatives.

74. A method for treating an inflammatory mucocutaneous disorder, said method comprising:
contacting said inflammatory mucocutaneous disorder with a concentrated inflammation modifier, wherein said concentrated inflammation modifier comprises a polar fraction obtained by chromatographic separation of a fat, oil, or wax obtained from a plant, animal, or mineral source.

75. The method of claim 74, wherein said inflammatory mucocutaneous disorder is a member selected from the group consisting of psoriasis, eczema and atopic dermatitis.
